(19) United States
(12) Patent Application Publication
(10) Pub. No.: US 2009/0048295 A1
(43) Pub. Date: Feb. 19, 2009

(54) SUBSTITUTED 5,6,7,8-TETRAHYDROQUINOLINE DERIVATIVES, COMPOSITIONS, AND METHODS OF USE THEREOF

(76) Inventors: Joseph Kent Barbay, Flourtown, PA (US); Wei He, Audubon, PA (US); Yong Gong, Warrington, PA (US); Jian Li, Lansdale, PA (US); Jean Van Wauwe, Beerse (BE); Mieke Buntinx, Hasselt (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/190,239

(22) Filed: Aug. 12, 2008

Related U.S. Application Data
(60) Provisional application No. 60/955,473, filed on Aug. 13, 2007.

(51) Int. Cl. A61K 31/47; C07D 215/233; A61P 17/06; A61P 25/28

(52) U.S. Cl. 514/312; 546/153; 546/171; 514/311

(57) ABSTRACT

Substituted 5,6,7,8-tetrahydroquinoline derivatives, which are C5a receptor modulators, compositions containing these derivatives, and methods of their use for the prevention and treatment of conditions, including, inter alia, immune and inflammatory diseases and conditions, are disclosed.

### CROSS REFERENCE TO RELATED APPLICATIONS

This application claims the benefit of U. S. Provisional Application 60/955,473, filed on Aug. 13, 2007, which is incorporated by reference herein in its entirety.

### FIELD OF THE INVENTION

The present invention is directed to substituted 5,6,7,8-tetrahydroquinoline derivatives, which are C5a receptor modulators, compositions containing these derivatives, and methods of their use for the prevention and treatment of conditions, including, inter alia, immune and inflammatory diseases and conditions, including sepsis, rheumatoid arthritis, psoriasis, inflammatory bowel disease, immune complex diseases, systemic lupus erythematosus, lupus nephritis, Alzheimer's disease, ischemia/reperfusion injury, multiple sclerosis, myasthenia gravis, glomerulonephritis, chronic graft rejection, gingivitis, asthma, dermatitis, Guillain-Barre syndrome, and combinations thereof.

### BACKGROUND OF THE INVENTION

The complement system is part of the humoral innate immune system. Its activation leads to the production of the anaphylatoxin C5a. This 74-amino acid-long N-terminal fragment of complement factor C5 is a potent pro-inflammatory mediator that increases vascular permeability, induces edema formation, and contracts smooth muscle. C5a also causes the extravasation of neutrophils, mast cells and monocytbes to extravascular sites, induces the release of cytokines (IL-1, IL-6, IL-8 and TNF), chemokines, lysosomal enzymes, and products of arachidonic acid metabolism and histamine from leukocytes, facilitates the formation of superoxide anions, and is also capable of augmenting humoral and cell-mediated immune responses. C5a exerts these activities by binding to the G-protein-coupled C5a receptor (C5aR or CD88) expressed on the membrane of myeloid cells including neutrophils, monocytes, basophils, eosinophils, dendritic cells, but also on glial cells, cerebellar granule cells, vascular endothelial cells, smooth muscle cells, and cells of liver and lung. These processes play a critical role in the clearance of invading pathogens. However, excessive or inappropriate generation of C5a might cause tissue damage associated with a number of immune and inflammatory diseases/manifestations including, for example, sepsis, rheumatoid arthritis, psoriasis, inflammatory bowel disease, immune complex diseases, systemic lupus erythematosus, Alzheimer's disease, and ischemia/reperfusion injury. Animal models of inflammation provide considerable evidence of the potentially adverse contribution of C5a. Conversely, beneficial effects are noted in a number of disease models after immunoneutralization of C5a, blockade of C5aR by C5aR antibodies or C5aR antagonists, such as C5a mutants, synthetic cyclic peptide C5aR antagonists, and other small molecule C5aR antagonists. Beneficial effects of C5aR antagonism include for example:
(1) protection from inflammatory responses in septic shock and immune complex disease models (primates and rodents);
(2) reduction of ischemia/reperfusion injury;
(3) inhibition of endotoxin- or C5a-induced neutropenia; and
(4) resistance to the development of antibody-induced arthritis in C5aR-deficient mice (which develop normally and show no phenotypic abnormalities).

Novel, small molecule C5aR antagonists are needed to selectively target the C5a-C5aR function of the complement system in diseases with excessive or inappropriate complement activation, including immune and inflammatory diseases and conditions, including sepsis, rheumatoid arthritis, psoriasis, inflammatory bowel disease, immune complex diseases, systemic lupus erythematosus, Alzheimer's disease and ischemia/reperfusion injury. The present invention focuses on novel compounds and compositions containing these compounds, directed to these and other important uses.

The disclosures of each patent, patent application, and publication cited or described in this document are hereby incorporated herein by reference, in their entirety.

### SUMMARY OF THE INVENTION

The present invention is directed to substituted 5,6,7,8-tetrahydroquinoline derivatives, useful as C5a receptor modulators, compositions containing these derivatives, and methods of their use for the prevention and treatment of conditions, including, inter alia, immune and inflammatory diseases and conditions, including sepsis, rheumatoid arthritis, psoriasis, inflammatory bowel disease, immune complex diseases, systemic lupus erythematosus, lupus nephritis, Alzheimer's disease, ischemia/reperfusion injury, multiple sclerosis, myasthenia gravis, glomerulonephritis, chronic graft rejection, gingivitis, asthma, dermatitis, Guillain-Barre syndrome, myocardial infarct, pancreatitis, cystic fibrosis, atherosclerosis, fibrosis, allergies, diabetes type I, and combinations thereof.

The present invention is directed to compounds of formula (I)

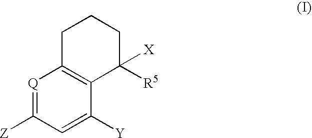

wherein:

Q is selected from the group consisting of N and N⁺O³¹ ;

X is selected from the group consisting of —NR¹R², —OR³, and —SR³;

Y is selected from the group consisting of —OR⁷, —SR⁷, R⁸, halo and hydrogen;

Z is selected from the group consisting of C₆₋₁₅ aryl and a 5 to 15 membered heteroaryl; wherein the C₆₋₁₅aryl or 5 to 15 membered heteroaryl is optionally substituted with 1 to 3 R⁴ substituents;

R⁵ is hydrogen or C₁₋₆ alkyl;

R¹ is selected from the group consisting of C₁₋₁₀ alkyl, C₃₋₁₀cycloalky, C₆₋₁₅aryl, C₆₋₁₅arylC₁₋₆alkyl, a 5 to 15 membered heteroaryl, a 5 to 15 membered heteroarylC₁₋₆alkyl and a 5 to 15 membered heterocyclyl; wherein the C₁₋₁₀alkyl, C₃₋₁₀cycloalkyl, C₆₋₁₅aryl, a 5 to 15 membered heteroaryl, or a 5 to 15 membered heterocyclyl, whether alone or as part of a substituent group, is optionally substituted with one to three R⁴ substituent;

wherein each R⁴ is independently selected from the group consisting of hydroxy, halo, cyano, C₁₋₁₀alkoxy, C₁₋₁₀ alkyl, halogenatedC₁₋₁₀alkyl, C₃₋₁₀cycloalkyl, aminoC₁₋₁₀alkyl, C₁₋₁₀alkylamino, di(C₁₋₁₀)alkylamino, a 5 to 10 membered heterocyclyl, C₆₋₁₀aryl, a 5 to 10 membered heteroaryl, —NH₂, —C(O)R⁶, —C(O)OR⁶ and C₁₋₁₀ alkylthio; wherein said C₁₋₁₀ alkoxy, C₁₋₁₀alkyl, C₃₋₁₀ cycloalkyl, 5 to 10 membered heterocyclyl, C₆₋₁₀ aryl, 5 to 10 membered heteroaryl, is optionally substituted with hydroxy, halogenatedC₁₋₃alkyl, cyano, halo, C₁₋₃ alkyl, C₁₋₃alkoxy, —C(O)R⁶ or —C(O)OR⁶; and wherein R⁶ is selected from the group consisting of hydrogen, C₁₋₃ alkyl, and —NH₂;

R² is selected from the group consisting of hydrogen, —C(O)R⁸ and C₁₋₆alkyl; wherein the C₁₋₆alkyl is optionally substituted with 1 to 2 substituent independently selected from the group consisting of hydroxy, cyano, and halo;

alternatively, R¹ and R² are taken together with the nitrogen atom to which they are attached to form a five to seven membered heterocyclic ring which is optionally fused to a C₅₋₆aryl or a C₅₋₇ cycloalkyl; and wherein said C₅₋₆aryl or C₅₋₇ cycloalkyl is optionally substituted with 1 to 3 substituents independently selected from the group consisting of halo, cyano, C₁₋₃alkyl, C₁₋₃ alkoxy, halogenatedC₁₋₃alkyl, —C(O)R⁶ and —C(O)OR⁶;

R³ is selected from the group consisting of C₆₋₁₅ aryl, C₆₋₁₅arylC₁₋₆alkyl, C₆₋₁₅aryl-NH—C₁₋₆alkyl, a 5 to 15 membered heteroaryl, and a 5 to 15 membered heteroarylC₁₋₆alkyl; wherein the C₆₋₁₅ aryl or 5 to 15 membered heteroaryl, whether alone or as part of a substituent group is optionally substituted with 1 to 3 R⁴ substituents;

R⁷ is selected from the group consisting of C₁₋₁₀ alkyl and C₃₋₁₀ cycloalkyl; wherein the C₁₋₁₀ alkyl or C₃₋₁₀ cycloalkyl is optionally substituted with 1 to 3 R⁴ substituents;

R⁸ is selected from the group consisting of C₁₋₆ alkyl and C₃₋₇ cycloalkyl,

and enantiomers, stereoisomers, pro-drugs, solvates, and pharmaceutically acceptable salts thereof.

In yet other embodiments, the present invention is directed to compositions, comprising: (a) at least one compound of formula (I) or a pharmaceutically acceptable salt thereof; and (b) at least one pharmaceutically acceptable carrier.

In another embodiment, the invention is directed to methods of treating an immune or inflammatory disease or condition in a patient in need thereof, including sepsis, rheumatoid arthritis, psoriasis, inflammatory bowel disease, immune complex diseases, systemic lupus erythematosus, lupus nephritis, Alzheimer's disease, ischemia/reperfusion injury, multiple sclerosis, myasthenia gravis, glomerulonephritis, chronic graft rejection, gingivitis, asthma, dermatitis, Guillain-Barre syndrome, myocardial infarct, pancreatitis, cystic fibrosis, atherosclerosis, fibrosis, allergies, diabetes type I, and combinations thereof, comprising the step of: administering to said patient an effective amount of a compound of formula (I) or a pharmaceutically acceptable salt thereof.

### DETAILED DESCRIPTION OF THE INVENTION

The present invention is directed to substituted 5,6,7,8-tetrahydroquinoline derivatives of formula (I)

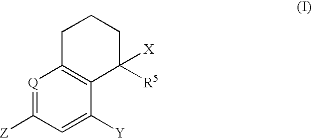

wherein Q, X, Y, Z and R⁵ are as herein defined, and enantiomers, stereoisomers, pro-drugs, solvates, and pharmaceutically acceptable salts thereof. The compounds of formula (I) are C5a receptor modulators. the present invention is further directed to compositions containing these derivatives, and methods of their use for the prevention and treatment of conditions, including, inter alia, immune and inflammatory diseases and conditions, including sepsis, rheumatoid arthritis, psoriasis, inflammatory bowel disease, immune complex diseases, systemic lupus erythematosus, lupus nephritis, Alzheimer's disease, ischemia/reperfusion injury, multiple sclerosis, myasthenia gravis, glomerulonephritis, chronic graft rejection, gingivitis, asthma, dermatitis, and/or Guillain-Barre syndrome.

The following definitions are provided for the full understanding of terms and abbreviations used in this specification.

As used herein and in the appended claims, the singular forms “a,” “an,” and “the” include the plural reference unless the context clearly indicates otherwise. Thus, for example, a reference to “an antagonist” includes a plurality of such antagonists, and a reference to “a compound” is a reference to one or more compounds and equivalents thereof known to those skilled in the art, and so forth.

“Halo,” as used herein, refers to chloro, bromo, fluoro, and iodo.

“C_{a-b}” (where a and b are integers) refers to a radical containing from a to b carbon atoms inclusive. For example, C₁₋₃ denotes a radical containing 1, 2 or 3 carbon atoms.

“Alkyl” whether used alone or as part of a substituent group refers to straight and branched carbon chains having 1 to 10 carbon atoms or any number within this range. Typical alkyl groups include, but are not limited to, methyl, ethyl, propyl, and butyl. In preferred embodiments, the alkyl group is C₁₋₈ alkyl, with C₁₋₃alkyl being particularly preferred. The term “alkoxy” refers to an —Oalkyl substituent group, wherein alkyl is defined supra.

“Halogenated alkyl” as used herein, refers to saturated branched or straight chain alkyl radical derived by removal of at least 1 hydrogen atom from the parent alkyl and substituting it with a halogen; the parent alkyl chain contains from 1 to 10 carbon atoms with 1 or more hydrogen atoms substituted with halogen atoms up to and including substitution of all hydrogen atoms with halogen. Preferred halogenated alkyl groups are fluorinated alkyls, including trifluoromethyl substituted alkyls and perfluorinated alkyls; more preferred fluorinated alkyls include trifluoromethyl, perfluoroethyl, and 1,1,2,2-tetrafluoroethyl; particularly preferred fluorinated alkyls are trifluoromethyl and 1,1,2,2-tetrafluoroethyl.

“Cycloalkyl”, as used herein, refers to saturated or partially unsaturated, monocyclic or polycyclic hydrocarbon rings of from 3 to 20 carbon atom members (preferably from 3 to 14, more preferably from 3 to 10 carbon atom members). Examples of such rings include, but are not limited to, cyclopropyl, cyclobutyl, cyclopentyl, cyclohexyl, cycloheptyl or adamantyl.

“Aryl,” as used herein, refers to an optionally substituted, mono-, di-, tri-, or other multicyclic aromatic ring system having from about 5 to about 15 carbon atoms (and all combinations and subcombinations of ranges and specific numbers of carbon atoms therein), with from about 6 to about 10 carbons being preferred. Particularly preferred are phenyl and naphthyl. Wherein the aryl is a multicyclic aromatic ring system, at least one of the rings is aromatic and one or more of the rings may be partially saturated or saturated.

“Heteroaryl,” as used herein, refers to an optionally substituted, mono-, di-, tri-, or other multicyclic aromatic ring system that includes at least one, and preferably from 1 to about 4 heteroatom ring members selected from sulfur, oxygen and nitrogen. In preferred embodiments, the heteroaryl group is a 5 to 15 membered heteroaryl, preferably a 5 to 10 membered heteroaryl, more preferably a 5 to 6 membered heteroaryl. Examples of heteroaryl groups include, but are not limited to, benzimidazolyl, benzisoxazolyl, benzofuranyl, benzopyrazolyl, benzothiadiazolyl, benzothiazolyl, benzothienyl, benzotriazolyl, benzoxazolyl, furanyl, furazanyl, furyl, imidazolyl, indazolyl, indolizinyl, indolinyl, indolyl, isobenzofuranyl, isoindolyl, isothiazolyl, isoxazolyl, oxazolyl, purinyl, pyrazinyl, pyrazolyl, pyridazinyl, pyridinyl, pyrimidinyl, pyrrolyl, quinolinyl, quinolyl, thiadiazolyl, thiazolyl, thiophenyl, or triazolyl. In certain embodiments, the heteroaryl may be partially saturated. Specific examples of the partially saturated heteroaryl are 1,2,3,4-tetrahydroquinolyl, etc.

The term “heterocyclyl” includes optionally substituted nonaromatic rings having carbon atoms and at least one heteroatom (O, S, N) or heteroatom moiety (SO₂, CO, CONH, COO) in the ring.

“Alkylthio,” as used herein, refers to the group R—S— where R is an alkyl group as defined herein.

The term “substituted” refers to a radical in which one or more hydrogen atoms are each independently replaced with the same or different substituent(s).

With reference to substituents, the term “independently” means that when more than one of such substituent is possible, such substituents may be the same or different from each other.

Throughout this disclosure, unless otherwise indicated, the terminal portion of the designated side chain is described first, followed by the adjacent functionality toward the point of attachment. Thus, for example, a “phenylC₁₋₆alkylaminocarbonylC₁₋₆alkyl” substituent refers to a group of the formula

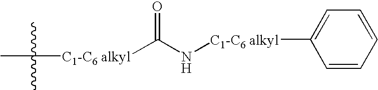

The abbreviations in the specification correspond to units of measure, techniques, properties, or compounds as follows:
° C.=degrees Celsius
AcOH=acetic acid
BSA=bovine serum albumin
cAMP=cyclic adenosine monophosphate
Ci=curie
Cmpd=compound
d=day/days
DCC=N,N′-dicyclohexylcarbodiimide
DCE=1,1-dichloroethane
DCM=dichloromethane
DIPEA=diisopropylethylamine
DMSO=dimethylsulfoxide
EDC=1-(3-dimethylaminopropyl)-3-ethylcarbodiimide hydrochloride
equiv=equivalents
EtOAc=ethyl acetate
EtOH=ethanol
Et₂O=diethyl ether
g=gram/grams
h=hour/hours
HBSS=Hank's balanced salt solution
HEPES=4-(2-hydroxyethyl)-1-piperazineethanesulfonic acid
HOBt or HOBT=hydroxybenzotriazole
HPLC=high-performance liquid chromatography
IC₅₀=half maximal inhibitory concentration
IL=Interleukin
IU=international unit
Kᵢ=inhibitory constant
L=liter
LAH=lithium aluminum hydride
LCMS=liquid chromatography-mass spectrometry
M=molar
m/z=mass-to-charge ratio
mCPBA=3-chloroperbenzoic acid
MeOH=methanol
mg=milligram/milligrams
MHz=megahertz
min=minute/minutes
mL=milliliters
mM=millimolar
mmol=millimoles
MS=mass spectrometry
MTBE=methyl t-butyl ether
NaOMe=sodium methoxide
nM=nanomolar
NMR=nuclear magnetic resonance
Pd₂(dba)₂=tris(dibenzylidene acetone)dipalladium(0)
Pd(PPh₃)₄=tetrakis(triphenylphosphine)palladium (0)
Pd(PPH₃)₂Cl₂=Bis(triphenylphosphine)palladium (II) chloride
pH=negative logarithm of hydrogen ion concentration
r.t.=room temperature
rpm=revolutions per minute
TEA=triethylamine
TFA=trifluoroacetic acid
THF=tetrahydrofuran
TNF=tumor necrosis factor
w=watts
μL=microliter
μM=micromolar
μmol=micromoles
μW=microwave

In the context of this disclosure, a number of terms shall be utilized:

The term “subject” or “patient” refers to an animal, preferably the human species, that is treatable with the compound, compositions, and/or methods of the present invention. The term “subject” or “subjects” is intended to refer to both the male and female gender unless one gender is specifically indicated. Accordingly, the term “patient” comprises a human that may benefit from prevention and treatment of conditions, including, inter alia, immune and inflammatory diseases and conditions, including sepsis, rheumatoid arthritis, psoriasis, inflammatory bowel disease, immune complex diseases, systemic lupus erythematosus, lupus nephritis, Alzheimer's disease, ischemia/reperfusion injury, multiple sclerosis, myasthenia gravis, glomerulonephritis, chronic graft rejection, gingivitis, asthma, dermatitis, Guillain-Barre syndrome, myocardial infarct, pancreatitis, cystic fibrosis, atherosclerosis, fibrosis, allergies, diabetes type I, and combinations thereof.

The term “administering,” as used herein, means either directly administering a compound or composition of the present invention, or administering a prodrug, derivative or analog which will form an equivalent amount of the active compound or substance within the body.

The term “treatment” as used herein includes preventative (e.g., prophylactic), curative or palliative treatment and “treating” as used herein also includes preventative, curative and palliative treatment.

The term “effective amount,” as used herein, refers to an amount effective, at dosages, and for periods of time necessary, to achieve the desired result with respect to prevention and treatment of conditions, including, inter alia, immune and inflammatory diseases and conditions, including sepsis, rheumatoid arthritis, psoriasis, inflammatory bowel disease, immune complex diseases, systemic lupus erythematosus, lupus nephritis, Alzheimer's disease, ischemia/reperfusion injury, multiple sclerosis, myasthenia gravis, glomerulonephritis, chronic graft rejection, gingivitis, asthma, dermatitis, Guillain-Barre syndrome, myocardial infarct, pancreatitis, cystic fibrosis, atherosclerosis, fibrosis, allergies, diabetes type I, and combinations thereof.

It will be appreciated that the effective amount of components of the present invention will vary from patient to patient not only with the particular compound, component or composition selected, the route of administration, and the ability of the components (alone or in combination with one or more combination drugs) to elicit a desired response in the individual, but also with factors such as the disease state or severity of the condition to be alleviated, hormone levels, age, sex, weight of the individual, the state of being of the patient, and the severity of the pathological condition being treated, concurrent medication or special diets then being followed by the particular patient, and other factors which those skilled in the art will recognize, with the appropriate dosage ultimately being at the discretion of the attendant physician. Dosage regimens may be adjusted to provide the improved therapeutic response. An effective amount is also one in which any toxic or detrimental effects of the components are outweighed by the therapeutically beneficial effects.

Preferably, the compounds of the present invention are administered at a dosage and for a time such that immune and inflammatory diseases and conditions are prevented, alleviated, or eliminated partially or completely. For example, for an afflicted patient, compounds of formula (I), or a pharmaceutically acceptable salt thereof, may be administered, preferably, at a dosage of from about 0.1 mg/day to about 2500 mg/day, dosed one to four times a day, preferably dosed one or two times daily, more preferably from about 0.1 mg/day to about 1500 mg/day, more preferably 1 mg/day to about 200 mg/day and most preferably from about 1 mg/day to 100 mg/day for a time sufficient to reduce and/or substantially eliminate the symptom or condition of the immune and inflammatory diseases and conditions.

As used herein, the term “modulation” refers to the capacity to either enhance or inhibit a functional property of a biological activity or process, for example, receptor binding or signaling activity. Such enhancement or inhibition may be contingent on the occurrence of a specific event, such as activation of a signal transduction pathway and/or may be manifest only in particular cell types. The modulator is intended to comprise any compound that binds to a receptor to form a complex, and is preferably an antagonist or a inverse agonist.

As used herein, the term “antagonist” refers to any agent that inhibits, suppresses, represses, or decreases a specific activity, such as C5a receptor signalling. The term “antagonist” is intended to comprise any compound that exhibits a partial, complete, competitive and/or inhibitory effect on the C5a receptor, thus diminishing or blocking, preferably diminishing, some or all of the biological effects of C5a.

The terms “component,” “composition of compounds,” “compound,” “drug,” or “pharmacologically active agent” or “active agent” or “medicament” are used interchangeably herein to refer to a compound or compounds or composition of matter which, when administered to a subject (e.g., human) induces a desired pharmacological and/or physiologic effect by local and/or systemic action.

In one embodiment, the present invention is directed to compounds of formula (I):

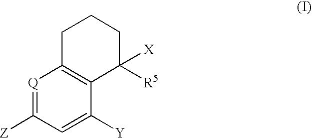

and enantiomers, stereoisomers, pro-drugs, solvates, and pharmaceutically acceptable salts thereof;

wherein:

X is —NR¹R², —OR³, or —SR³;

Y is —OR⁷, —SR⁷, R⁸, halo, or hydrogen;

Z is C₆₋₁₅ aryl optionally substituted with 1-3 R⁴ or a 5 to 15 membered heteroaryl optionally substituted with 1-3 R⁴;

R¹ is C₁₋₁₀ alkyl optionally substituted with 1-3 R⁴, C₃₋₁₀ cycloalkyl optionally substituted with 1-3 R⁴, C₆₋₁₅ aryl optionally substituted with 1-3 R⁴, C₆₋₁₅arylC₁₋₆alkyl wherein said aryl group is optionally substituted with 1-3 R⁴, a 5 to 15 membered heteroaryl optionally substituted with 1-3 R⁴, a 5 to 15 membered heteroarylC₁₋₆alkyl wherein said heteroaryl group is optionally substituted with 1-3 R⁴, a 5 to 15 membered heterocyclyl optionally substituted with 1-3 R⁴;

R² is hydrogen, —C(O)R⁸, or C₁₋₆alkyl optionally substituted with 1-2 groups independently selected from the group consisting of hydroxyl, cyano, and halo;

or R¹ and R² are taken together with the nitrogen atom to which they are attached to form a five to seven membered heterocyclic ring which is optionally fused to C₅₋₆aryl or a C₅₋₇ cycloalkyl wherein said C₅₋₆aryl or C₅₋₇ cycloalkyl is optionally substituted with 1-3 groups independently selected from the group consisting of halo, cyano, C₁₋₃alkyl, C₁₋₃ alkoxy, halogenatedC₁₋₃alkyl, —C(O)R⁶ and —C(O)OR⁶;

R³ is C₆₋₁₅ aryl optionally substituted with 1-3 R⁴, C₆₋₁₅arylC₁₋₆alkyl wherein said aryl group is optionally substituted with 1-3 R⁴, C₆₋₁₅aryl-NH—C₁₋₆alkyl wherein said aryl group is optionally substituted with 1-3 R⁴, a 5 to 15 membered heteroaryl optionally substituted with 1-3 R⁴, a 5 to 15 membered heteroarylC₁₋₆alkyl wherein said heteroaryl group is optionally substituted with 1-3 R⁴;

R⁴ is hydroxy, halo, cyano, C₁₋₁₀ alkoxy, C₁₋₁₀ alkyl, halogenatedC₁₋₁₀alkyl, C₃₋₁₀ cycloalkyl, aminoC₁₋₁₀alkyl, C₁₋₁₀alkylamino, di(C₁₋₁₀)alkylamino, a 5 to 10 membered heterocyclyl, C₆₋₁₀aryl, a 5 to 10 membered heteroaryl, —NH₂, —C(O)R⁶, —C(O)OR⁶, or C₁₋₁₀ alkylthio, wherein said C₁₋₁₀ alkoxy, C₁₋₁₀alkyl, C₃₋₁₀ cycloalkyl, 5 to 10 membered heterocyclyl, C₆₋₁₀ aryl, 5 to 10 membered heteroaryl, is optionally substituted with hydroxyl, halogenatedC₁₋₃alkyl, cyano, halo, C₁₋₃ alkyl, C₁₋₃ alkoxy, —C(O)R⁶ or —C(O)OR⁶;

R⁵ is hydrogen or C₁₋₆ alkyl;

R⁶ is hydrogen, C₁₋₃ alkyl, or —NH₂;

R⁷ is C₁₋₁₀ alkyl optionally substituted with 1-3 R⁴, or C₃₋₁₀ cycloalkyl optionally substituted with 1-3 R⁴;

R⁸ is C₁₋₆ alkyl, or C₃₋₇ cycloalkyl, and

Q is N or N-oxide.

In certain embodiments, R¹ is selected from the group consisting of C₁₋₁₀ alkyl, C₃₋₁₀cycloalky, C₆₋₁₅aryl, C₆₋₁₅arylC₁₋₆alkyl, a 5 to 15 membered heteroaryl, a 5 to 15 membered heteroarylC₁₋₆alkyl and a 5 to 15 membered heterocyclyl; wherein the C₁₋₁₀alkyl, C₃₋₁₀cycloalkyl, C₆₋₁₅aryl, a 5 to 15 membered heteroaryl, or a 5 to 15 membered heterocyclyl, whether alone or as part of a substituent group, is optionally substituted with one to three R⁴ substituent;

wherein each R⁴ is independently selected from the group consisting of hydroxy, halo, cyano, C₁₋₁₀alkoxy, C₁₋₁₀ alkyl, halogenatedC₁₋₁₀alkyl, C₃₋₁₀cycloalkyl, aminoC₁₋₁₀alkyl, C₁₋₁₀alkylamino, di(C₁₋₁₀)alkylamino, a 5 to 10 membered heterocyclyl, C₆₋₁₀aryl, a 5 to 10 membered heteroaryl, —NH₂, —C(O)R⁶, —C(O)OR⁶ and C₁₋₁₀ alkylthio; wherein said C₁₋₁₀ alkoxy, C₁₋₁₀alkyl, C₃₋₁₀ cycloalkyl, 5 to 10 membered heterocyclyl, C₆₋₁₀ aryl, 5 to 10 membered heteroaryl, is optionally substituted with hydroxy, halogenatedC₁₋₃alkyl, cyano, halo, C₁₋₃ alkyl, C₁₋₃ alkoxy, —C(O)R⁶ or —C(O)OR⁶; and wherein R⁶ is selected from the group consisting of hydrogen, C₁₋₃ alkyl, and —NH₂.

In certain embodiments, R² is selected from the group consisting of hydrogen, —C(O)R⁸ and C₁₋₆alkyl; wherein the C₁₋₆alkyl is optionally substituted with 1 to 2 substituent independently selected from the group consisting of hydroxy, cyano, and halo.

In certain embodiments, R¹ and R² are taken together with the nitrogen atom to which they are attached to form a five to seven membered heterocyclic ring which is optionally fused to a C₅₋₆aryl or a C₅₋₇ cycloalkyl; and wherein said C₅₋₆aryl or C₅₋₇ cycloalkyl is optionally substituted with 1 to 3 substituents independently selected from the group consisting of halo, cyano, C₁₋₃alkyl, C₁₋₃ alkoxy, halogenatedC₁₋₃alkyl, —C(O)R⁶ and —C(O)OR⁶.

In certain embodiments, R³ is selected from the group consisting of C₆₋₁₅ aryl, C₆₋₁₅arylC₁₋₆alkyl, C₆₋₁₅aryl-NH—C₁₋₆alkyl, a 5 to 15 membered heteroaryl, and a 5 to 15 membered heteroarylC₁₋₆alkyl; wherein the C₆₋₁₅ aryl or 5 to 15 membered heteroaryl, whether alone or as part of a substituent group is optionally substituted with 1 to 3 R⁴ substituents.

In certain embodiments, R⁷ is selected from the group consisting of C₁₋₁₀ alkyl and C₃₋₁₀ cycloalkyl; wherein the C₁₋₁₀ alkyl or C₃₋₁₀ cycloalkyl is optionally substituted with 1 to 3 R⁴ substituents.

In certain embodiments, R⁸ is selected from the group consisting of C₁₋₆ alkyl and C₃₋₇ cycloalkyl.

In certain preferred embodiments X is NR¹R².

In certain preferred embodiments, R¹ is selected from C₆₋₁₀aryl, C₆₋₁₀arylC₁₋₃alkyl, a 5 to 10 membered heteroaryl, a 5 to 10 membered heteroarylC₁₋₃alkyl, and a 5 to 10 membered heterocyclyl, wherein each R₁ is optionally substituted 1-3 groups independently selected from C₁₋₆alkyl, halogenatedC₁₋₆alkyl, hydroxyl substituted C₁₋₆alkyl, halo, hydroxyl, C₁₋₆alkoxy, cyano, —NH₂, NH₂C₁₋₃alkyl, —C(O)OR⁶, and —C(O)R⁶.

In certain preferred embodiments R¹ is selected from naphthyl, phenyl, naphthylenylmethyl, furanylmethyl, indanyl, phenylpropyl, phenethyl, benzyl, thiophenylmethyl, indolyl, tetrahydroisoquinolyl, tetrahydronaphthyl, pyridyl, and 1,2,3,4-tetrahydroquinolyl, each of which may optionally be substituted with 1-3 R⁴ groups, preferably 1-3 R⁴ groups independently selected from C₁₋₆alkyl, halogenated C₁₋₆alkyl, hydroxyl substituted C₁₋₆alkyl, halo, hydroxyl, C₁₋₆alkoxy, cyano, —NH₂, NH₂C₁₋₃alkyl, —C(O)OR⁶ and —C(O)R⁶.

In certain preferred embodiments, R² is selected from the group consisting of hydrogen, —C(O)C₁₋₃alkyl, and C₁₋₃alkyl optionally substituted 1-2 groups independently selected from the group consisting of hydroxyl, cyano, and halo. Preferably, R² is selected from hydrogen, and C₁₋₃alkyl optionally substituted with hydroxyl or cyano. In certain further preferred embodiments, R² is selected from hydrogen and C₁₋₃alkyl.

In certain embodiments, X is selected from the group consisting of —NR¹R², —OR³, and —SR³.

In certain preferred embodiments, X is NR¹R², wherein R¹ is selected from naphthyl, phenyl, naphthylenylmethyl, furanylmethyl, indanyl, phenylpropyl, phenethyl, benzyl, thiophenylmethyl, indolyl, tetrahydroisoquinolyl, tetrahydronaphthyl, pyridyl, and 1,2,3,4-tetrahydroquinolyl, each of which may optionally be substituted with 1-3 R⁴; and wherein R² is selected from hydrogen, C₁₋₃alkyl, —CH₂CH₂OH, and —CH₂CH₂CH₂OH. In certain preferred embodiments, NR¹R²forms a five to seven membered heterocyclic ring which is optionally fused to C₅₋₆ aryl or C₅₋₇ cycloalkyl wherein said C₅₋₆ aryl or C₅₋₇ cycloalkyl is optionally substituted with 1-2 members independently selected from C₁₋₃alkyl, halogenatedC₁₋₃alkyl, C₁₋₃alkoxy, —C(O)OR⁶, —C(O)R⁶, halo, and cyano. In other preferred embodiments, X is NR¹R², wherein NR¹R² is isoindolinyl, indolinyl, tetrahydroisoquinolyl, or 1,2,3,4-tetrahydroquinolyl each of which may optionally be substituted with 1-2 members independently selected from C₁₋₃alkyl, halogenatedC₁₋₃alkyl, C₁₋₃alkoxy, —C(O)OR⁶, —C(O)R⁶, halo, and cyano.

In other preferred embodiments, X is OR³. In certain preferred embodiments, R³ is selected from C₆₋₁₀ aryl, C₆₋₁₀arylC₁₋₃alkyl, C₆₋₁₀aryl-NH—C₁₋₃alkyl, wherein each C₆₋₁₀aryl is optionally substituted with 1-2 groups independently selected from C₁₋₃alkyl, hydroxyl substituted C₁₋₃alkyl, C₁₋₃alkoxy, —C(O)OR⁶, and —C(O)R⁶. In certain preferred embodiments, X is OR³, wherein R³ is naphthyl, phenyl, or benzyl, each of which may optionally be substituted with 1-3 R⁴, preferably optionally substituted with 1-2 groups independently selected from C₁₃alkyl, hydroxyl substituted C₁₋₃alkyl, C₁₋₃alkoxy, —C(O)OR⁶, and —C(O)R⁶.

In especially preferred embodiments, X is selected from the group consisting of N-(3-trifluoromeythyl-6-hydroxymethyl-phenyl)-amino-, N-(3-chloro-6-hydroxymethyl-phenyl)-amino-, N-(2-hydroxyethyl-phenyl)-amino-, N-(3-fluoro-6-hydroxymethyl-phenyl)-amino-, N-(naphth-1-yl)-N-(hydroxyethyl)-amino-, N-(2-hydroxymethyl-phenyl)-amino-, N-methyl-N-(naphth-1-yl)-amino-, N-ethyl-N-(naphth-1-yl)-amino-, N-(2-ethylphenyl)-amino-, 2-methyl-5-methoxy-phenoxy-, 2-(1,2,3,4-tetrahydro-isoquinolinyl), N-(2-chloro-5-methoxy-phenyl)-amino-, N-(2-carboxy-phenyl)-amino-, N-(2,5-dimethoxy-phenyl)-amino-, N-(2,5-dimethyl-phenyl)-amino-, N-methyl-N-(4-methoxy-naphth-1-yl)-amino-, N-methyl-N-(2,5-dimethyl-phenyl)-amino-, 2-(7-chloro-1,2,3,4-tetrahydro-isoquinolinyl), N-methyl-N-(1,2,3,4-tetrahydronaphth-1-yl)-amino-, 2-(8-methoxycarbonyl-1,2,3,4-tetrahydro-isoquinolinyl), 2-(7-fluoro-1,2,3,4-tetrahydro-isoquinolinyl), N-(2-methylphenyl)-amino-, N-methyl-N-(2-chloro-5-methoxy-phenyl)-amino-, N-methyl-N-(4-chloro-naphth-1-yl)-amino-, N-(cyanomethyl)-N-(naphth-1-yl)-amino-, N-(1-methyl-5-fluoro-phenyl)-amino-, N-methyl-N-(2-methyl-5-chloro-phenyl)-amino-, N-(2-methyl-5-chloro-phenyl)-amino-, N-methyl-N-(1,2,3,4-tetrahydro-quinolin-1-yl)-amino-, 2-(5-chloro-1,2,3,4-tetrahydro-isoquinolinyl), N-(2-methyl-5-methoxy-phenyl)-amino-, N-(2-methyl-4-methoxy-phenyl)-amino-, 2-(hydroxyethyl)-phenoxy-, 1-(1,2,3,4-tetrahydro-quinolinyl), N-(2-methyl-5-phenyl-phenyl)-amino-, N-(3-methoxy-phenyl)-amino-, N-methyl-N-(2-methyl-5-methoxy-phenyl )-amino-, N-(4-hydroxymethyl-pyrid-3-yl )-amino-, N-methyl-N-(4-indanyl)-amino-, N-(2-cyanophenyl)-amino-, N-(2-aminomethyl-phenyl)-amino-, N-ethyl-N-(2,3-dimethylphenyl)-amino-, N-(methylcarbonyl)-N-(naphth-1-yl)-amino-, N-methyl-N-(2-methyl-4-methoxy-phenyl)-amino-, 2-(8-trifluoromethyl-1,2,3,4-tetrahydro-isoquinolinyl), N-(2-methyl-5-trifluoromethyl-phenyl)-amino-, N-methyl-N-(2-methylphenyl)-amino-, N-methyl-N-(2-methyl-naphth-1-yl)-amino-, N-methyl-N-(2,5-dimethoxy-phenyl)-amino-, N-methyl-N-phenyl-amino-, N-ethyl-N-(1,2,3,4-tetrahydro-naphth-5-yl)-amino- and N-methyl-N-(6-methoxy-naphth-1-yl)-amino-.

In certain embodiments, X is selected from the group consisting of N-(2-hydroxy-ethyl-phenyl )-amino-, N-(2-hydroxymethyl-5-trifluoromethyl-phenyl)-amino-, N-(2-hydroxymethyl-5-chloro-phenyl)-amino, (R)—N-ethyl-N-(napthy-1-yl)-amino-, N-(2-hydroxyethyl-phenyl)-amino-, N-ethyl-N-(naphth-1-yl)-amino-, N-(2-hydroxymethyl-4-fluoro-phenyl)-amino-, N-(2-hydroxymethyl-4-chloro-phenyl)-amino-, N-(2-methyl-5-methoxy-phenyl)-amino-, N-(hydroxyethyl)-N-(naphth-1-yl)-amino-, N-(ethyl)-N-(naphth-1-yl)-amino-, N-(2-hydroxymethyl-phenyl)-amino-, N-(2-methyl-4-methoxy-phenyl)-amino-, N-methyl-N-(naphth-1-yl)-amino-, N-methyl-N-(6-methoxy-naphth-1-yl)-amino-, N-ethyl-N-(5,6,7,8-tetrahydro-naphth-1-yl)-amino-, 2-(8-trifluoromethyl-1,2,3,4-tetrahydroisoquinolinyl), N-(2-methyl-5-trifluoromethyl-phenyl)-amino-, 2-methyl-5-methoxy-phenyl-oxy-, 2-(1,2,3,4-tetrahydroisoquinolinyl), N-(2-chloro-5-methoxy-phenyl)-amino-, N-(2-carboxyphenyl)-amino-, N-(2,5-dimethoxy-phenyl)-amino-, N-(2,5-dimethylphenyl)-amino-, N-methyl-N-(4-methoxy-naphth-1-yl)-amino-, N-methyl-N-(2-methyl-5-methoxy-phenyl)-amino-, N-methyl-N-(2,5-dimethyl-phenyl)-amino-, N-(2-ethyl-phenyl)-amino-, N-methyl-N-(2-methylphenyl)-amino-, (R)—N-methyl-N—(S)-1,2,3,4-tetrahydronaphth-1-yl)-amino-, 2-(8-methoxycarbonyl-1,2,3,4-tetrahydro-isoquinolinyl)-, 2-(7-fluoro-1,2,3,4-tetrahydro-isoquinolinyl)-, N-(2-methylphenyl)-amino-, N-methyl-N-(2-chloro-5-methoxy-phenyl)-amino-, N-methyl-N-(4-chloro-naphth-1-yl)-amino-, N-(cyanomethyl)-N-(naphth-1-y)-amino-, N-(2-methyl-5-fluoro-phenyl)-amino-,2-(7-chloro-1,2,3,4-tetrahydro-isoquinolinyl), N-methyl-N-(2-methyl-naphth-1-yl)-amino-, N-(2-methyl-5-chloro-phenyl)-amino-, N-methyl-N-(1,2,3,4-tetrahydroquinolin-1-yl), 2-(5-chloro-1,2,3,4-tetrahydroisoquinolinyl), (S)—N-methyl-N—(R)-(1,2,3,4-tetrahydro-naphth-1-yl)-amino-, 2-hydroxyethyl-phenyl-oxy-, 1-(1,2,3,4-tetrahydro-quinolinyl), N-(2-methyl-5-phenyl-phenyl)-amino-, N-methyl-N-(2-methyl-5-chloro-phenyl)-amino-, N-methyl-(2,5-dimethoxy-phenyl)-amino-, N-(4-hydroxymethyl-pyrid-3-yl)-amino, N-methyl-N-(indan-4-yl), N-(2-cyano-phenyl-amino-, N-(2-aminomethyl-phenyl)-amino-, N-ethyl-N-(2,3-dimethyl-phenyl)-amino-, (S)—N-methyl-N—(S)-(1,2,3,4-tetrahydro-naphth-1-yl)-amino-, N-(methylcarbonyl)-N-(naphth-1-yl)-amino-, N-methyl-N-(2-methyl-4-methoxy-phenyl)-amino-, N-(3-methoxy-phenyl)-amino-, N-methyl-N-phenyl-amino-, (R)—N-methyl-N—(R)-(1,2,3,4-tetrahydro-naphth-1-yl)-amino-, N-ethyl-N-benzyl-amino-, benzyloxy-, N-methyl-N-(phenylethyl)-amino-, N-(indan-2-yl)-amino-, 2-(2,3-dihydro-1H-isoindolyl), N-methyl-N-(naphth-1-yl-methyl)-amino-, N-methyl-N-(phenyl-n-propyl)-amino, N-methyl-N-(2-furyl-methyl)-amino-, 1-(2,3-dihydro-1H-indolyl), N-methyl-N-(2-thienyl-methyl)-amino-, N-methyl-N-(indan-2-yl)-amino-, 2-(7-cyano-1,2,3,4-tetrahydro-isoquinolinyl), 2-(7-methoxy-carbonyl-1,2,3,4-tetrahydro-isoquinolinyl), 2-(6-methoxy-carbonyl -1,2,3,4-tetrahydro-isoquinolinyl), 2-(5-methoxy-carbonyl-1,2,3,4-tetrahydro-isoquinolinyl), 2-(7-methoxy-1,2,3,4-tetrahydro-isoquinolinyl), 2-(7-trifluoromethyl-1,2,3,4-tetrahydro-isoquinolinyl), 2-(6-trifluoromethyl-1,2,3,4-tetrahydro-isoquinolinyl), N-(naphth-1-yl)-amino, naphth-1-yl-oxy-, N-(naphth-1-yl)-amino-ethyl-oxy-, N-methyl-(N-(1-methyl-5-isopropyl-phenyl)-amino-, 2-methyl-5-isopropyl-phenyl-oxy-, N-(methyl)-N-(5-(2-methyl-1,2,3,4-tetrahydro-isoquinolinyl)-amino-, N-(methyl)-N-(indol-4-yl)-amino, N-(methyl)-N-(2,6-dimethyl-phenyl)-amino-, N-(methyl)-N-(2-methyl-3-chloro-phenyl)-amino-, N-(methyl)-N-(7-methoxy-naphth-1-yl)-amino-, N-(methyl)-N-(2-methyl-3-methoxy-phenyl)-amino-, N-(methyl)-N-(5-methoxy-naphth-1-yl)-amino-, N-(2-methyl-naphth-1-yl)-amino-, N-(2-trifluoromethyl-phenyl)-amino-, N-(4-indanyl)-amino-, N-(methyl)-N-(3-(4-methyl-biphenyl))-amino-, N-(methyl)-N-(benzyl)-amino-, N-(methyl)-N-(cyclohexyl)-amino-, N-(methyl)-N-(3-pyridyl-methyl)-amino-, N-pyrrolidinyl, 2-(6,7-dimethoxy-1,2,3,4-tetrahydro-isoquinolinyl), 2-(6-carboxy-1,2,3,4-tetrahydro-isoquinolinyl), 2-(5-carboxy-1,2,3,4-tetrahydro-isoquinolinyl), 2-(8-carboxy-1,2,3,4-tetrahydro-isoquinolinyl), 2-(7-carboxy-1,2,3,4-tetrahydro-isoquinolinyl), N-(naphth-1-yl)-amino-, N-(ethyl)-N-(naphth-1-yl)-amino-, N-(ethyl)-N-(quinazolin-4-yl)-amino-, N-(2-(4-methoxy-biphenyl))-amino-, N-(ethyl)-amino-, 2-(hydroxymethyl)-phenyl-oxy-, N-(2-methyl-5-methoxy-phenyl)-amino-, N-(2-methyl-4-hydroxymethyl-phenyl)-amino-, N-(3-hydroxymethyl-phenyl)-amino-, 2-formyl-5-methoxy-phenyl-oxy-, N-(2-methoxy-carbonyl-phenyl)-amino-, N-(2-amino-benzyl)-amino-, N-(2-methylcarbonyl-phenyl)-amino-, N-(2-methyl-5-hydroxy-phenyl)-amino, N-(2-aminocarbonyl-phenyl)-amino- and and 2-ethoxycarbonyl-phenyl-oxy-.

In certain embodiments, X is selected from the group consisting of N-(2-hydroxy-ethyl-phenyl )-amino-, N-(2-hydroxymethyl-5-trifluoromethyl-phenyl)-amino-, N-(2-hydroxymethyl-5-chloro-phenyl)-amino, (R)—N-ethyl-N-(napthy-1-yl)-amino-, N-(2-hydroxyethyl-phenyl)-amino-, N-ethyl-N-(naphth-1-yl)-amino-, N-(2-hydroxymethyl-4-fluoro-phenyl)-amino-, N-(2-hydroxymethyl-4-chloro-phenyl)-amino-, N-(2-methyl-5-methoxy-phenyl)-amino-, N-(hydroxyethyl)-N-(naphth-1-yl)-amino-, N-(2-hydroxymethyl-phenyl)-amino-, N-(2-methyl-4-methoxy-phenyl)-amino-, N-methyl-N-(naphth-1-yl)-amino-, N-methyl-N-(6-methoxy-naphth-1-yl)-amino-, N-ethyl-N-(5,6,7,8-tetrahydro-naphth-1-yl)-amino-, 2-(8-trifluoromethyl-1,2,3,4-tetrahydroisoquinolinyl), N-(2-methyl-5-trifluoromethyl-phenyl)-amino-, 2-methyl-5-methoxy-phenyl-oxy-, 2-(1,2,3,4-tetrahydroisoquinolinyl), N-(2-chloro-5-methoxy-phenyl)-amino-, N-(2-carboxyphenyl)-amino-, N-(2,5-dimethoxy-phenyl)-amino-, N-(2,5-dimethylphenyl)-amino-, N-methyl-N-(4-methoxy-naphth-1-yl)-amino-, N-methyl-N-(2-methyl-5-methoxy-phenyl)-amino-, N-methyl-N-(2,5-dimethyl-phenyl)-amino-, N-(2-ethyl-phenyl)-amino-, N-methyl-N-(2-methylphenyl)-amino-, (R)—N-methyl-N—(S)-1,2,3,4-tetrahydronaphth-1-yl)-amino-, 2-(8-methoxycarbonyl-1,2,3,4-tetrahydro-isoquinolinyl)-, 2-(7-fluoro-1,2,3,4-tetrahydro-isoquinolinyl)-, N-(2-methylphenyl)-amino-, N-methyl-N-(2-chloro-5-methoxy-phenyl)-amino-, N-methyl-N-(4-chloro-naphth-1-yl)-amino-, N-(cyanomethyl)-N-(naphth-1-y)-amino-, N-(2-methyl-5-fluoro-phenyl)-amino-, 2-(7-chloro-1,2,3,4-tetrahydro-isoquinolinyl), N-methyl-N-(2-methyl-naphth-1-yl)-amino-, N-(2-methyl-5-chloro-phenyl)-amino-, N-methyl-N-(1,2,3,4-tetrahydroquinolin-1-yl), N-(hydroxyethyl)-N-(naphth-1-yl)-amino-, 2-(5-chloro-1,2,3,4-tetrahydroisoquinolinyl), (S)—N-methyl-N—(R)-(1,2,3,4-tetrahydro-naphth-1-yl)-amino-, N-methyl-N-(naphth-1-yl)-amino-, N-(2-methyl-5-methoxy-phenyl)-amino-, 2-hydroxyethyl-phenyl-oxy-, 1-(1,2,3,4-tetrahydro-quinolinyl), N-(2-methyl-5-phenyl-phenyl)-amino-, N-methyl-N-(2-methyl-5-chloro-phenyl)-amino-, N-methyl-(2,5-dimethoxy-phenyl)-amino-, N-(4-hydroxymethyl-pyrid-3-yl)-amino, N-methyl-N-(indan-4-yl), N-(2-cyano-phenyl-amino-, N-(2-aminomethyl-phenyl)-amino-, N-ethyl-N-(2,3-dimethyl-phenyl)-amino-, (S)—N-methyl-N—(S)-(1,2,3,4-tetrahydro-naphth-1-yl)-amino-, N-(methylcarbonyl)-N-(naphth-1-yl)-amino-, N-methyl-N-(2-methyl-4-methoxy-phenyl)-amino-, N-(3-methoxy-phenyl)-amino-, N-methyl-N-phenyl-amino- and(R)—N-methyl-N—(R)-(1,2,3,4-tetrahydro-naphth-1-yl)-amino-.

In certain embodiments, X is selected from the group consisting of N-(2-hydroxy-ethyl-phenyl )-amino-, N-(2-hydroxymethyl-5-trifluoromethyl-phenyl)-amino-, N-(2-hydroxymethyl-5-chloro-phenyl)-amino, (R)—N-ethyl-N-(napthy-1-yl)-amino-, N-(2-hydroxyethyl-phenyl)-amino-, N-ethyl-N-(naphth-1-yl)-amino-, N-(2-hydroxymethyl-4-fluoro-phenyl)-amino-, N-(2-hydroxymethyl-4-chloro-phenyl)-amino-, N-(2-methyl-5-methoxy-phenyl)-amino-, N-(hydroxyethyl)-N-(naphth-1-yl)-amino-, N-(2-hydroxymethyl-phenyl)-amino-, N-methyl-N-(naphth-1-yl)-amino-, N-methyl-N-(6-methoxy-naphth-1-yl)-amino-, N-ethyl-N-(5,6,7,8-tetrahydro-naphth-1-yl)-amino-, 2-(8-trifluoromethyl-1,2,3,4-tetrahydroisoquinolinyl), N-(2-methyl-5-trifluoromethyl-phenyl)-amino-, 2-methyl-5-methoxy-phenyl-oxy-, 2-(1,2,3,4-tetrahydroisoquinolinyl), N-(2-chloro-5-methoxy-phenyl)-amino-, N-(2-carboxyphenyl)-amino-, N-(2,5-dimethoxy-phenyl)-amino-, N-(2,5-dimethylphenyl)-amino-, N-methyl-N-(4-methoxy-naphth-1-yl)-amino-, N-methyl-N-(2-methyl-5-methoxy-phenyl)-amino-, N-methyl-N-(2,5-dimethyl-phenyl)-amino-, N-(2-ethyl-phenyl)-amino-, N-methyl-N-(2-methylphenyl)-amino-, (R)—N-methyl-N—(S)-1,2,3,4-tetrahydronaphth-1-yl)-amino-, 2-(8-methoxycarbonyl-1,2,3,4-tetrahydro-isoquinolinyl)-, 2-(7-fluoro-1,2,3,4-tetrahydro-isoquinolinyl)-, N-(2-methylphenyl)-amino- and N-methyl-N-(2-chloro-5-methoxy-phenyl)-amino-.

In certain preferred embodiments Y is C₁₋₁₀ alkoxy, C₃₋₁₀cycloalkoxy, C₁₋₆ alkyl, C₁₋₁₀alkylthio, halo, phenylC₁₋₃alkoxy, or hydrogen. In certain further preferred embodiments, Y is C₁₋₃alkoxy, C₁₋₃alkyl, C₁₋₃alkylthio, or C₃₋₇cycloalkoxy, more preferably C₁₋₃ alkoxy or C₁₋₃alkylthio. In certain further preferred embodiments, Y is hydrogen, methoxy, ethoxy, isopropoxy, cyclopentyloxy, benzyloxy, methylthio, ethyl, or chloro. In certain further preferred embodiments, Y is methoxy, ethoxy, isopropoxy, cyclopentyloxy, or methylthio.

In certain embodiment, Y is selected from the group consisting of —OR⁷, —SR⁷, R⁸, halo and hydrogen. In certain embodiments, Y is selected from the group consisting of hydrogen, hydroxy, chloro, methylthio, ethyl, methoxy, ethoxy, isopropoxy, benzyloxy, cyclopentyl-oxy and dimethylamino-ethoxy. In certain embodiments, Y is selected from the group consisting of isopropoxy, methoxy, cyclopentyl-oxy, ethoxy, methylthio, ethyl, benzyloxy, chloro and dimethyl-amino-ethoxy. In certain embodiments, Y is selected from the group consisting of isopropoxy, methoxy, cyclopentyloxy, ethoxy, methylthio, ethyl and benzyloxy.

In certain embodiments, Z is C₆₋₁₀aryl optionally substituted with 1-3 R⁴ or a 5 to 10 membered heteroaryl optionally substituted with 1-3 R⁴. In certain embodiments, Z is phenyl optionally substituted with 1-3 members independently selected from C₁₋₃alkyl, C₁₋₃alkoxy, and halo, preferably Z is phenyl substituted with 1-3 members independently selected from methyl, ethyl, methoxy, and ethoxy. In certain preferred embodiments Z is phenyl substituted at the 2 position relative to the point of attachment, and preferably at the 2 and 6 position relative to the point of attachment. In certain preferred embodiments, Z is phenyl, naphthyl, or benzo[1,3]dioxolyl, each of which may be optionally substituted with 1-3 R⁴. In especially preferred embodiments, Z is 2,6-diethyl-phenyl, 2,6-dimethyl-phenyl, 4-methoxy-2,6-dimethyl-phenyl, 2,6-dimethoxy-phenyl, 2-methoxy-6-chloro-phenyl, 2-methyl-phenyl, 2-ethyl-phenyl, 2-isopropyl-phenyl, 2-fluorophenyl, or naphthyl.

In certain embodiments, Z is selected from the group consisting of C₆₋₁₅ aryl and a 5 to 15 membered heteroaryl; wherein the C₆₋₁₅aryl or 5 to 15 membered heteroaryl is optionally substituted with 1 to 3 R⁴ substituents.

In certain embodiments, Z is selected from the group consisting of naphth-1-yl, phenyl, 2-fluorophenyl, 2-methylphenyl, 2-ethylphenyl, 2-isopropylphenyl, 2,6-dimethyl-phenyl, 2,6-dimethoxy-phenyl, 2,6-diethyl-phenyl, 2-chloro-6-methoxy-phenyl, 2,6-dimethyl-4-methoxy-phenyl, 2-(biphenyl), 3-thienyl, 3-pyridyl, 4-(3,5-dimethyl-isoxazolyl) and 2-(benzo[1,3]-(dioxolyl). In certain embodiments, Z is selected from the group consisting of 2,6-diethyl-phenyl, 2-chloro-6-methoxy-phenyl, 2,6-dimethyl-phenyl, 2,6-dimethyl-4-methoxy-phenyl, 2-methyl-phenyl, 2-ethyl-phenyl, 2-isopropyl-phenyl, 2,6-dimethoxy-phenyl, 2-fluoro-phenyl, 2,6-dimethyl-phenyl and naphth-1-yl. In certain embodiments, Z is selected from the group consisting of Z is selected from the group consisting of 2,6-diethyl-phenyl, 2-chloro-6-methoxy-phenyl, 2,6-dimethyl-phenyl, 2,6-dimethyl-4-methoxy-phenyl, 2-methyl-phenyl and 2-ethyl-phenyl. In certain embodiments, Z is selected from the group consisting of 2-fluorophenyl, 2-methylphenyl, 2-ethylphenyl, 2-isopropylphenyl, 2.6-diethylphenyl, 2,6-dimethyl-4-methoxy-phenyl, 2-chloro-6-methoxy-phenyl and naphth-1-yl.

In certain embodiments, R⁵ is selected from the group consisting of hydrogen and C₁₋₆ alkyl. In certain embodiments, R⁵ is selected from the group consisting of hydrogen, methyl and n-propyl. In certain embodiment, R⁵ is selected from the group consisting of hydrogen and n-propyl. In certain embodiments, R⁵ is hydrogen. In certain preferred embodiments, R₅ is —CH₃, —CH₂CH₃, or —CH₂CH₂CH₃.

In certain preferred embodiments, Q is selected from the group consisting of N and N⁺O⁻. In certain preferred embodiments, Q is N. In certain preferred embodiments, Q is N⁺O⁻.

In certain preferred embodiments, X is selected form the group consisting of (R)—N-ethyl-N-(napth-1-yl)-amino-, (R)—N-methyl-N—(R)-(1,2,3,4-tetrahydro-naphth-1-yl)-amino-, (R)—N-methyl-N—(S)-1,2,3,4-tetrahydronaphth-1-yl)-amino-, (S)—N-methyl-N—(R)-(1,2,3,4-tetrahydro-naphth-1-yl)-amino-, (S)—N-methyl-N—(S)-(1,2,3,4-tetrahydro-naphth-1-yl)-amino-, 1-(1,2,3,4-tetrahydro-quinolinyl), 2-(1,2,3,4-tetrahydro-isoquinolinyl), 2-(5-chloro-1,2,3,4-tetrahydro-isoquinolinyl), 2-(7-chloro-1,2,3,4-tetrahydro-isoquinolinyl), 2-(7-fluoro-1,2,3,4-tetrahydro-isoquinolinyl)-, 2-(8-methoxycarbonyl-1,2,3,4-tetrahydro-isoquinolinyl)-, 2-(8-trifluoromethyl-1,2,3,4-tetrahydroisoquinolinyl), 2-hydroxyethyl-phenyl-oxy-, 2-methyl-5-methoxy-phenyl-oxy-, N-(2,5-dimethoxy-phenyl)-amino-, N-(2,5-dimethyl-phenyl)-amino-, N-(2-aminomethyl-phenyl)-amino-, N-(2-carboxyphenyl)-amino-, N-(2-chloro-5-methoxy-phenyl)-amino-, N-(2-cyano-phenyl)-amino-, N-(2-ethyl-phenyl)-amino-, N-(2-hydroxyethyl-phenyl)-amino-, N-(2-hydroxymethyl-4-chloro-phenyl)-amino-, N-(2-hydroxymethyl-4-fluoro-phenyl)-amino-, N-(2-hydroxymethyl-5-chloro-phenyl)-amino, N-(2-hydroxymethyl-phenyl)-amino-, N-(2-methyl-4-methoxy-phenyl)-amino-, N-(2-methyl-5-chloro-phenyl)-amino-, N-(2-methyl-5-fluoro-phenyl)-amino-, N-(2-methyl-5-methoxy-phenyl)-amino-, N-(2-methyl-5-phenyl-phenyl)-amino-, N-(2-methyl-5-trifluoromethyl-phenyl)-amino-, N-(2-methylphenyl)-amino-, N-(2-hydroxymethyl-5-trifluoromethyl-phenyl)-amino-, N-(3-methoxy-phenyl)-amino-, N-(4-hydroxymethyl-pyrid-3-yl)-amino, N-(cyanomethyl)-N-(naphth-1-y)-amino-, N-(hydroxyethyl)-N-(naphth-1-yl)-amino-, N-(ethyl)-N-(naphth-1-yl)-amino-, N-(methylcarbonyl)-N-(naphth-1-yl)-amino-, N-ethyl-N-(2,3-dimethyl-phenyl)-amino-, N-ethyl-N-(5,6,7,8-tetrahydro-naphth-1-yl)-amino-, N-methyl-(2,5-dimethoxy-phenyl)-amino-, N-methyl-N-(1,2,3,4-tetrahydroquinolin-1-yl), N-methyl-N-(2,5-dimethyl-phenyl)-amino-, N-methyl-N-(2-chloro-5-methoxy-phenyl)-amino-, N-methyl-N-(2-methyl-4-methoxy-phenyl)-amino-, N-methyl-N-(2-methyl-5-chloro-phenyl)-amino-, N-methyl-N-(2-methyl-5-methoxy-phenyl)-amino-, N-methyl-N-(2-methyl-naphth-1-yl)-amino-, N-methyl-N-(2-methylphenyl)-amino-, N-methyl-N-(4-chloro-naphth-1-yl)-amino-, N-methyl-N-(4-methoxy-naphth-1-yl)-amino-, N-methyl-N-(6-methoxy-naphth-1-yl)-amino-, N-methyl-N-(indan-4-yl), N-methyl-N-(naphth-1-yl)-amino- and N-methyl-N-phenyl -amino-.

In certain preferred embodiments, R⁵ is selected from the group consisting of hydrogen and n-propyl. In certain preferred embodiments, R₅ is H.

In certain preferred embodiments, Y is selected from the group consisting of chloro, ethyl, methoxy, ethoxy, isopropoxy, methylthio, benzyloxy, cyclopentyl-oxy and dimethylamino-ethoxy.

In certain preferred embodiments, Z is selected from the group consisting of 2-fluorophenyl, 2-methylphenyl, 2-ethylphenyl, 2-isopropylphenyl, 2.6-diethylphenyl, 2,6-dimethyl-4-methoxy-phenyl, 2-chloro-6-methoxy-phenyl and naphth-1-yl.

In an embodiment, X is other than carboxy-substituted 1,2,3,4-tetrahydro-isoquinolin-2-yl. In another embodiment, X is other than 2-(5-carboxy-1,2,3,4-tetrahydro-isoquinolinyl), 2-(6-carboxy-1,2,3,4-tetrahydro-isoquinolinyl), 2-(7-carboxy-1,2,3,4-tetrahydro-isoquinolinyl) and 2-(8-carboxy-1,2,3,4-tetrahydro-isoquinolinyl).

In especially preferred embodiments, the compound of formula (I) is selected from:

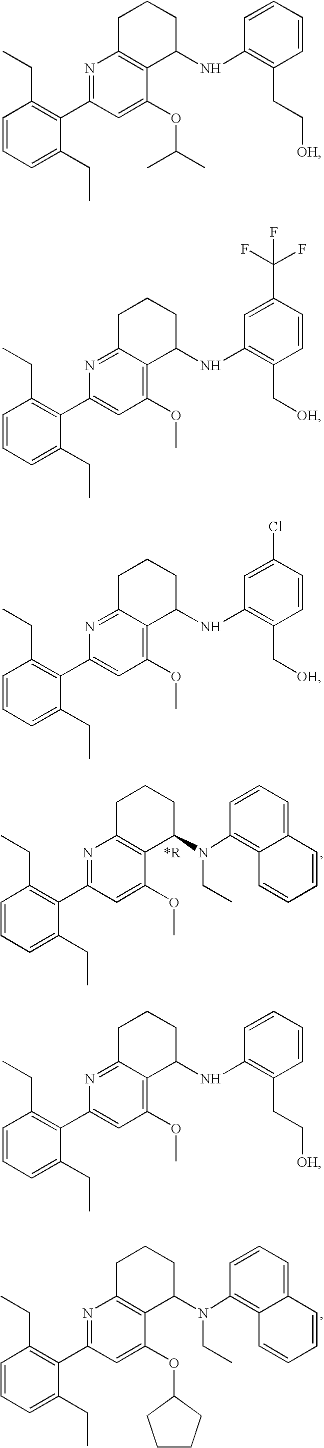

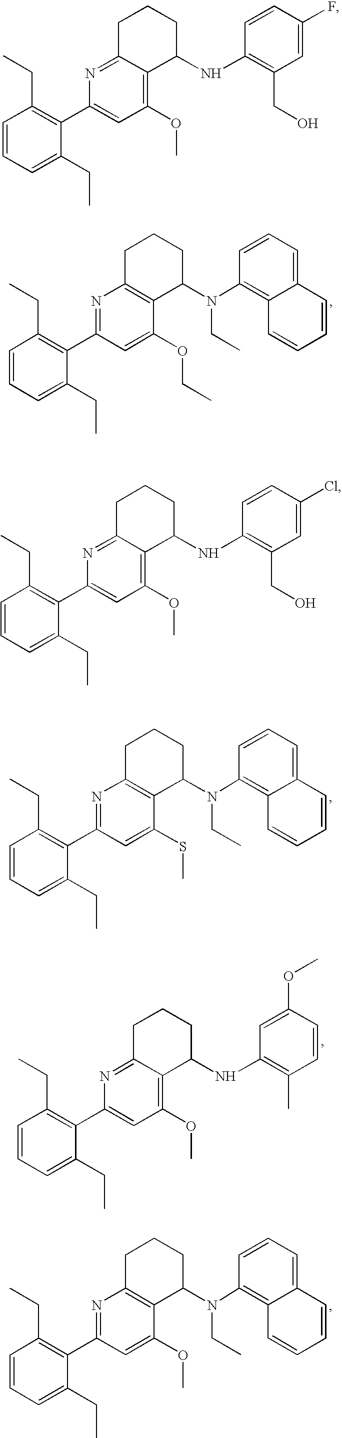

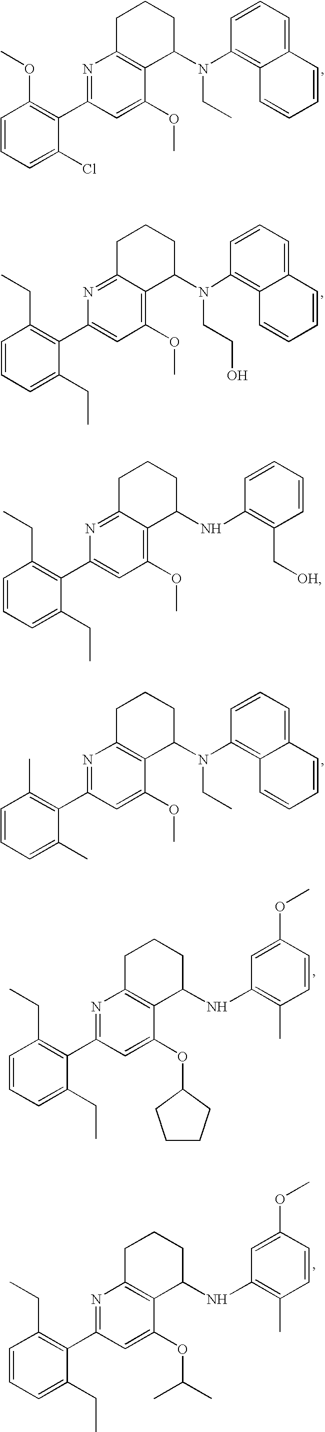

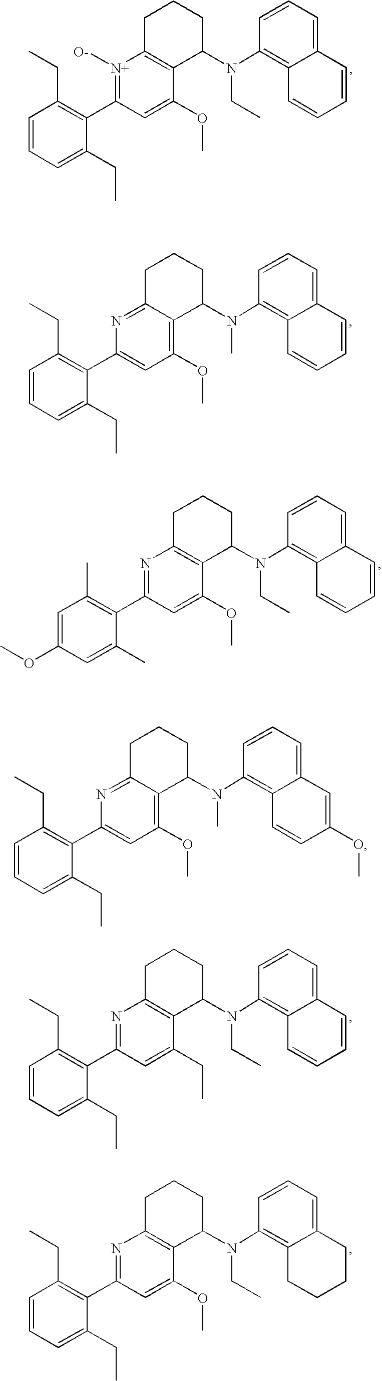

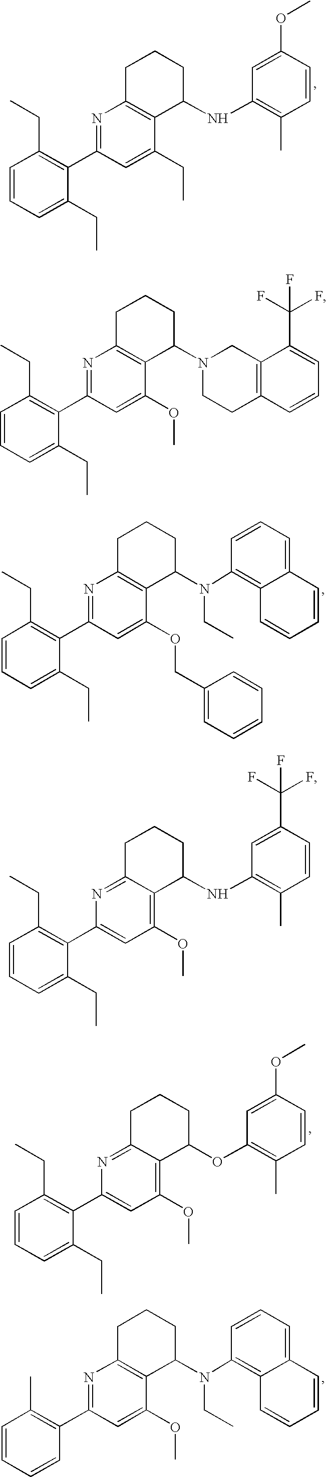

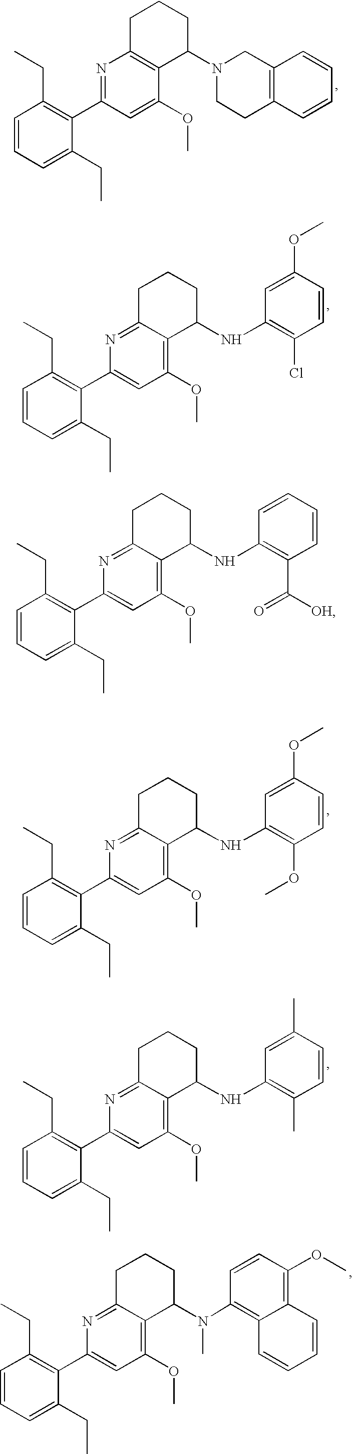

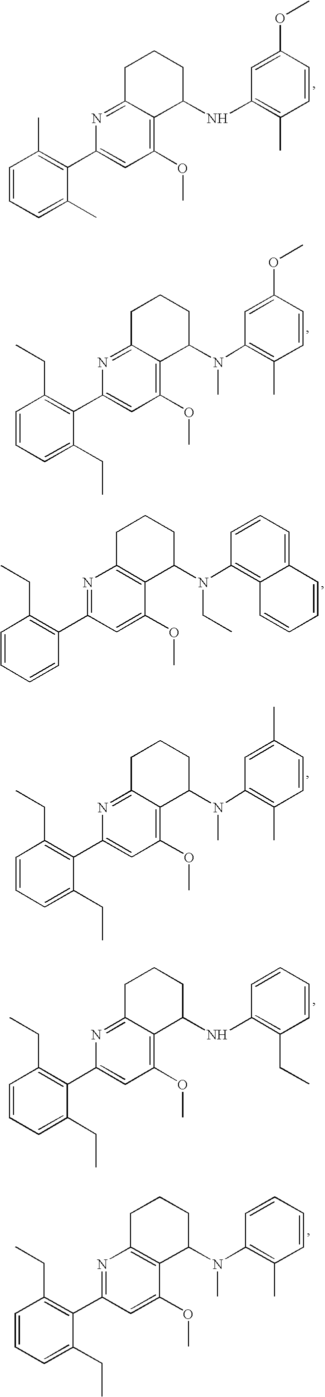

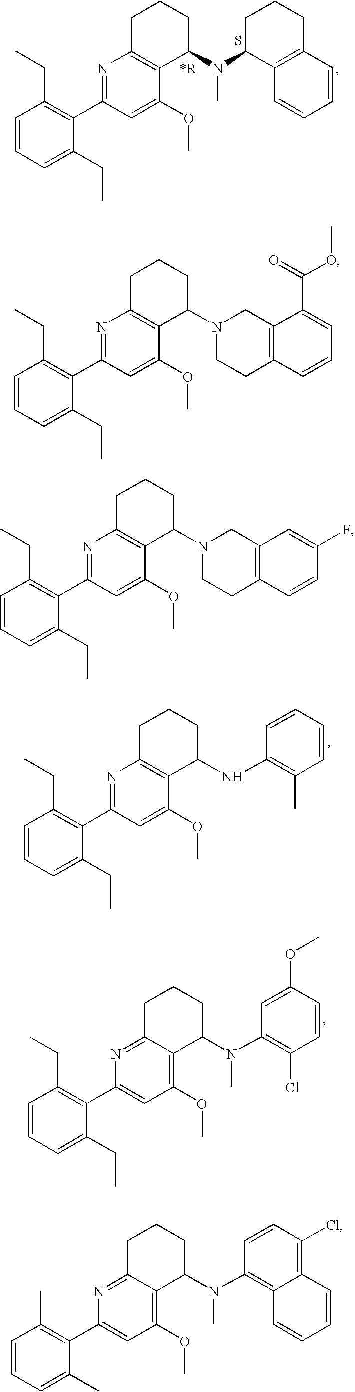

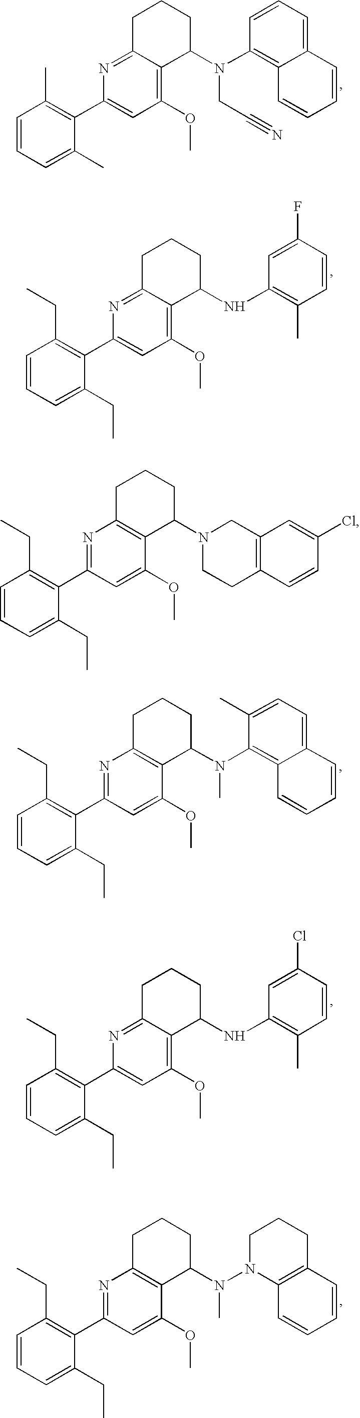

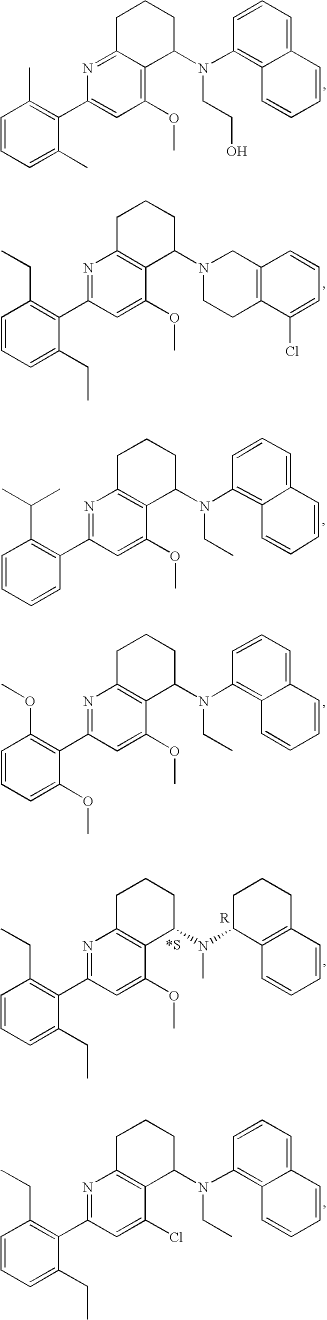

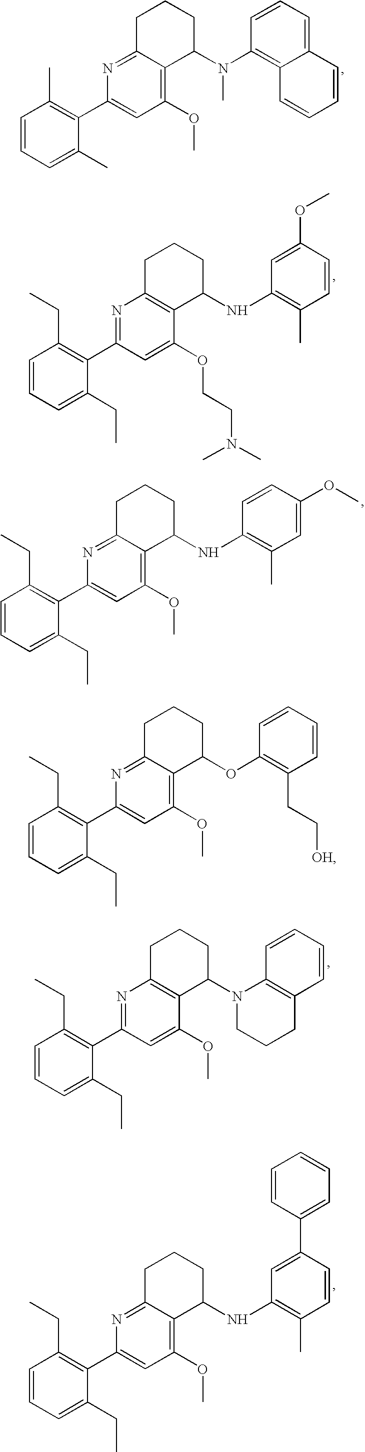

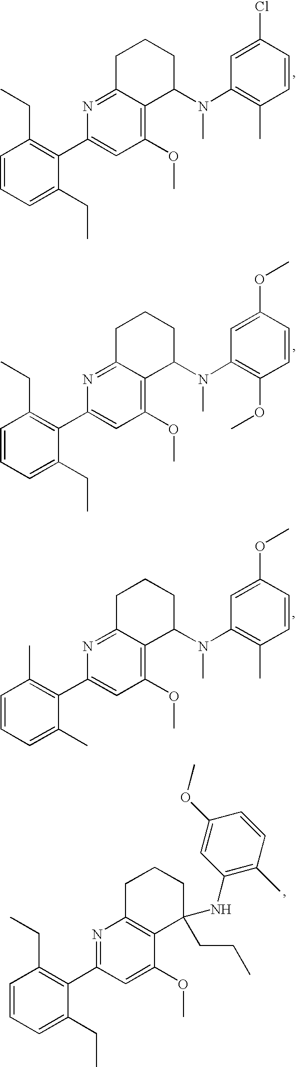

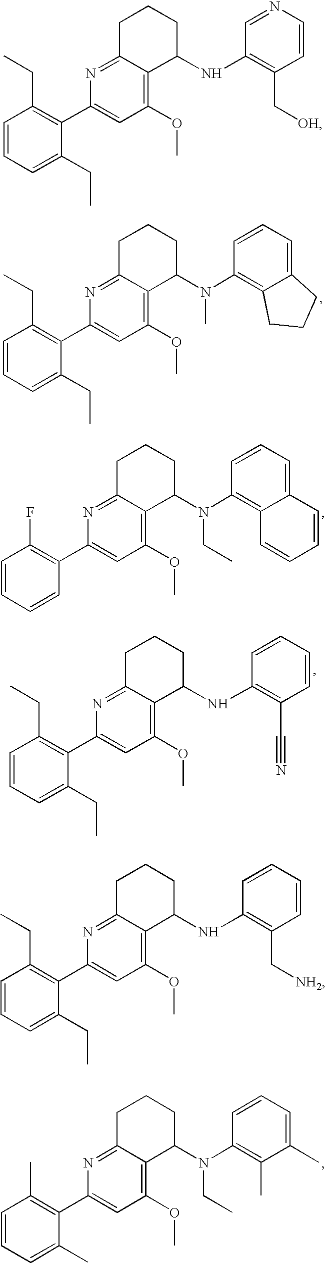

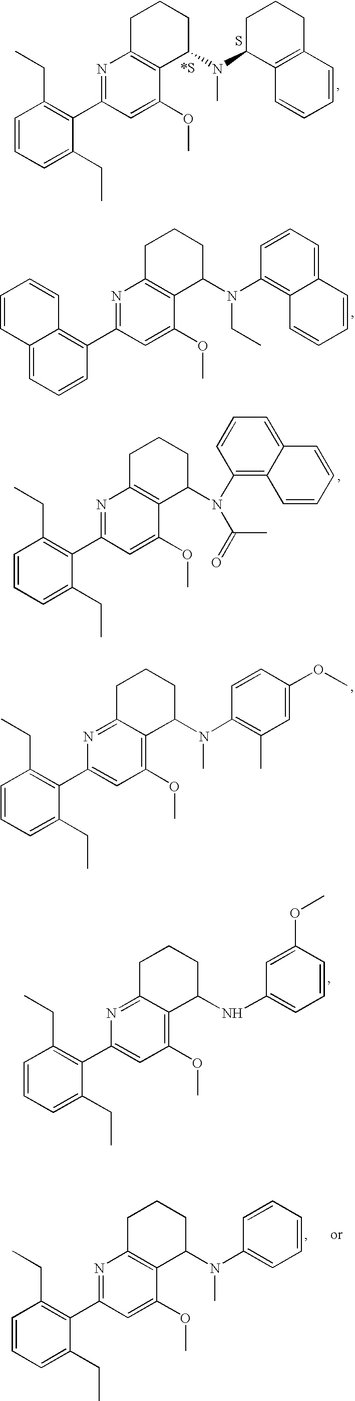

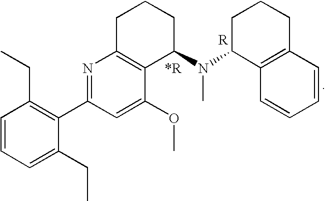

In certain preferred embodiments, the present invention is directed to any single compound or subset of compound selected from the representative compounds listed in Table 1, below.

TABLE 1
Representative Compounds of Formula (I)

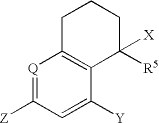

| ID | | | | | |
|---|---|---|---|---|---|
| No. | Q | X | R⁵ | Y | Z |
| 1 | N | N-(2-hydroxy-ethyl- | H | iso- | 2,6-diethyl- |
| | | phenyl)-amino- | | propoxy | phenyl |
| 2 | N | N-(2-hydroxymethyl-5- | H | methoxy | 2,6-diethyl- |
| | | trifluoromethyl-phenyl)- | | | phenyl |
| | | amino- | | | |
| 3 | N | N-(2-hydroxymethyl-5- | H | methoxy | 2,6-diethyl- |
| | | chloro-phenyl)-amino- | | | phenyl |
| 4 | N | (R)-N-ethyl-N-(napth-1- | H | methoxy | 2,6-diethyl- |
| | | yl)-amino- | | | phenyl |
| 5 | N | N-(2-hydroxyethyl- | H | methoxy | 2,6-diethyl- |
| | | phenyl)-amino- | | | phenyl |
| 6 | N | N-ethyl-N-(naphth-1- | H | cyclo- | 2,6-diethyl- |
| | | amino- | | pentyl- | phenyl |
| | | | | oxy | |
| 7 | N | N-(2-hydroxymethyl-4- | H | methoxy | 2,6-diethyl- |
| | | fluoro-phenyl)-amino- | | | phenyl |
| 8 | N | N-ethyl-N-(naphth-1- | H | ethoxy | 2,6-diethyl- |
| | | yl)-amino- | | | phenyl |
| 9 | N | N-(2-hydroxymethyl-4- | H | methoxy | 2,6-diethyl- |
| | | chloro-phenyl)-amino- | | | phenyl |
| 10 | N | N-ethyl-N-(naphth-1- | H | methylthio | 2,6-diethyl- |
| | | yl)-amino- | | | phenyl |
| 11 | N | N-(2-methyl-5-methoxy- | H | methoxy | 2,6-diethyl- |
| | | phenyl)-amino- | | | phenyl |
| 12 | N | N-ethyl-N-(naphth-1- | H | methoxy | 2,6-diethyl- |
| | | yl)-amino- | | | phenyl |
| 13 | N | N-ethyl-N-(naphth-1- | H | methoxy | 2-chloro-6- |
| | | yl)-amino- | | | methoxy- |
| | | | | | phenyl |
| 14 | N | N-(hydroxyethyl)-N- | H | methoxy | 2,6-diethyl- |
| | | (naphth-1-yl)-amino- | | | phenyl |
| 15 | N | N-(2-hydroxymethyl- | H | methoxy | 2,6-diethyl- |
| | | phenyl)-amino- | | | phenyl |
| 16 | N | N-(ethyl)-N-(naphth-1- | H | methoxy | 2,6- |
| | | yl)-amino- | | | dimethyl- |
| | | | | | phenyl |
| 17 | N | N-(2-methyl-5-methoxy- | H | cyclo- | 2,6-diethyl- |
| | | phenyl)-amino- | | pentyl- | phenyl |
| | | | | oxy | |
| 18 | N | N-(2-methyl-5-methoxy- | H | iso- | 2,6-diethyl- |
| | | phenyl)-amino- | | propoxy | phenyl |
| 19 | N⁺ | N-ethyl-N-(naphth-1-yl)- | H | methoxy | 2,6-diethyl- |
| | O⁻ | amino- | | | phenyl |
| 20 | N | N-methyl-N-(naphth-1- | H | methoxy | 2,6-diethyl- |
| | | yl)-amino- | | | phenyl |
| 21 | N | N-ethyl-N-(naphth-1-yl)- | H | methoxy | 2,6- |
| | | amino- | | | dimethyl-4- |
| | | | | | methoxy- |
| | | | | | phenyl |
| 22 | N | N-methyl-N-(6-methoxy- | H | methoxy | 2,6-diethyl- |
| | | naphth-1-yl)-amino- | | | phenyl |
| 23 | N | N-ethyl-N-(naphth-1-yl)- | H | ethyl | 2,6-diethyl- |
| | | amino- | | | phenyl |
| 24 | N | N-ethyl-N-(5,6,7,8- | H | methoxy | 2,6-diethyl- |
| | | tetrahydro-naphth-1-yl)- | | | phenyl |
| | | amino- | | | |
| 25 | N | N-(2-methyl-5-methoxy- | H | ethyl | 2,6-diethyl- |
| | | phenyl)-amino- | | | phenyl |
| 26 | N | 2-(8-trifluoromethyl- | H | methoxy | 2,6-diethyl- |
| | | 1,2,3,4- | | | phenyl |
| | | tetrahydroisoquinolinyl) | | | |
| 27 | N | N-ethyl-N-(naphth-1-yl)- | H | benzyloxy | 2,6-diethyl- |
| | | amino- | | | phenyl |
| 28 | N | N-(2-methyl-5- | H | methoxy | 2,6-diethyl- |
| | | trifluoromethyl-phenyl)- | | | phenyl |
| | | amino- | | | |
| 29 | N | 2-methyl-5-methoxy- | H | methoxy | 2,6-diethyl- |
| | | phenyl-oxy- | | | phenyl |
| 30 | N | N-ethyl-N-(naphth-1-yl)- | H | methoxy | 2-methyl- |
| | | amino- | | | phenyl |
| 31 | N | 2-(1,2,3,4- | H | methoxy | 2,6-diethyl- |
| | | tetrahydroisoquinolinyl) | | | phenyl |
| 32 | N | N-(2-chloro-5-methoxy- | H | methoxy | 2,6-diethyl- |
| | | phenyl)-amino- | | | phenyl |
| 33 | N | N-(2-carboxyphenyl)- | H | methoxy | 2,6-diethyl- |
| | | amino- | | | phenyl |
| 34 | N | N-(2,5-dimethoxy- | H | methoxy | 2,6-diethyl- |
| | | phenyl)-amino- | | | phenyl |
| 35 | N | N-(2,5-dimethylphenyl)- | H | methoxy | 2,6-diethyl- |
| | | amino- | | | phenyl |
| 36 | N | N-methyl-N-(4-methoxy- | H | methoxy | 2,6-diethyl- |
| | | naphth-1-yl)-amino- | | | phenyl |
| 37 | N | N-(2-methyl-5-methoxy- | H | methoxy | 2,6- |
| | | phenyl)-amino- | | | dimethyl- |
| | | | | | phenyl |
| 38 | N | N-methyl-N-(2-methyl- | H | methoxy | 2,6-diethyl- |
| | | 5-methoxy-phenyl)- | | | phenyl |
| | | amino- | | | |
| 39 | N | N-ethyl-N-(naphth-1-yl)- | H | methoxy | 2-ethyl- |
| | | amino- | | | phenyl |
| 40 | N | N-methyl-N-(2,5- | H | methoxy | 2,6-diethyl- |
| | | dimethyl-phenyl)-amino- | | | phenyl |
| 41 | N | N-(2-ethyl-phenyl)- | H | methoxy | 2,6-diethyl- |
| | | amino- | | | phenyl |
| 42 | N | N-methyl-N-(2- | H | methoxy | 2,6-diethyl- |
| | | methylphenyl)-amino- | | | phenyl |
| 43 | N | (R)-N-methyl-N-(S)- | H | methoxy | 2,6-diethyl- |
| | | 1,2,3,4- | | | phenyl |
| | | tetrahydronaphth-1-yl)- | | | |
| | | amino- | | | |
| 44 | N | 2-(8-methoxycarbonyl- | H | methoxy | 2,6-diethyl- |
| | | 1,2,3,4-tetrahydro- | | | phenyl |
| | | isoquinolinyl)- | | | |
| 45 | N | 2-(7-fluoro-1,2,3,4- | H | methoxy | 2,6-diethyl- |
| | | tetrahydro- | | | phenyl |
| | | isoquinolinyl)- | | | |
| 46 | N | N-(2-methylphenyl)- | H | methoxy | 2,6-diethyl- |
| | | amino- | | | phenyl |
| 47 | N | N-methyl-N-(2-chloro-5- | H | methoxy | 2,6-diethyl- |
| | | methoxy-phenyl)- | | | phenyl |
| | | amino- | | | |
| 48 | N | N-methyl-N-(4-chloro- | H | methoxy | 2,6- |
| | | naphth-1-yl)-amino- | | | dimethyl- |
| | | | | | phenyl |
| 49 | N | N-(cyanomethyl)-N- | H | methoxy | 2,6- |
| | | (naphth-1-y)-amino- | | | dimethyl- |
| | | | | | phenyl |
| 50 | N | N-(2-methyl-5-fluoro- | H | methoxy | 2,6-diethyl- |
| | | phenyl)-amino- | | | phenyl |
| 51 | N | 2-(7-chloro-1,2,3,4- | H | methoxy | 2,6-diethyl- |
| | | tetrahydro-isoquinolinyl) | | | phenyl |
| 52 | N | N-methyl-N-(2-methyl- | H | methoxy | 2,6-diethyl- |
| | | naphth-1-yl)-amino- | | | phenyl |
| 53 | N | N-(2-methyl-5-chloro- | H | methoxy | 2,6-diethyl- |
| | | phenyl)-amino- | | | phenyl |
| 54 | N | N-methyl-N-(1,2,3,4- | H | methoxy | 2,6-diethyl- |
| | | tetrahydroquinolin-1-yl) | | | phenyl |
| 55 | N | N-(hydroxyethyl)-N- | H | methoxy | 2,6- |
| | | (naphth-1-yl)-amino- | | | dimethyl- |
| | | | | | phenyl |
| 56 | N | 2-(5-chloro-1,2,3,4- | H | methoxy | 2,6-diethyl- |
| | | tetrahydroisoquinolinyl) | | | phenyl |
| 57 | N | N-ethyl-N-(naphth-1-yl)- | H | methoxy | 2-isopropyl- |
| | | amino- | | | phenyl |
| 58 | N | N-ethyl-N-(naphth-1-yl)- | H | methoxy | 2,6- |
| | | amino- | | | dimethoxy- |
| | | | | | phenyl |
| 59 | N | (S)-N-methyl-N-(R)- | H | methoxy | 2,6-diethyl- |
| | | (1,2,3,4-tetrahydro- | | | phenyl |
| | | naphth-1-yl)-amino- | | | |
| 60 | N | N-ethyl-N-(naphth-1-yl)- | H | chloro | 2,6-diethyl- |
| | | amino- | | | phenyl |
| 61 | N | N-methyl-N-(naphth-1- | H | methoxy | 2,6- |
| | | yl)-amino- | | | dimethyl- |
| | | | | | phenyl |
| 62 | N | N-(2-methyl-5-methoxy- | H | dimethyl- | 2,6-diethyl- |
| | | phenyl)-amino- | | amino- | phenyl |
| | | | | ethoxy | |
| 63 | N | N-(2-methyl-4-methoxy- | H | methoxy | 2,6-diethyl- |
| | | phenyl)-amino- | | | phenyl |
| 64 | N | 2-hydroxyethyl-phenyl- | H | methoxy | 2,6-diethyl- |
| | | oxy- | | | phenyl |
| 65 | N | 1-(1,2,3,4-tetrahydro- | H | methoxy | 2,6-diethyl- |
| | | quinolinyl) | | | phenyl |
| 66 | N | N-(2-methyl-5-phenyl- | H | methoxy | 2,6-diethyl- |
| | | phenyl)-amino- | | | phenyl |
| 67 | N | N-methyl-N-(2-methyl- | H | methoxy | 2,6-diethyl- |
| | | 5-chloro-phenyl)-amino- | | | phenyl |
| 68 | N | N-methyl-(2,5- | H | methoxy | 2,6-diethyl- |
| | | dimethoxy-phenyl)- | | | phenyl |
| | | amino- | | | |
| 69 | N | N-methyl-N-(2-methyl | H | methoxy | 2,6-diethyl- |
| | | 5-methoxy-phenyl)- | | | phenyl |
| | | amino- | | | |
| 70 | N | N-(2-methyl-5-methoxy- | n- | methoxy | 2,6-diethyl- |
| | | phenyl)-amino- | propyl | | phenyl |
| 71 | N | N-(4-hydroxymethyl- | H | methoxy | 2,6-diethyl- |
| | | pyrid-3-yl)-amino | | | phenyl |
| 72 | N | N-methyl-N-(indan-4-yl) | H | methoxy | 2,6-diethyl- |
| | | | | | phenyl |
| 73 | N | N-ethyl-N-(naphth-1-yl)- | H | methoxy | 2-fluoro- |
| | | amino- | | | phenyl |
| 74 | N | N-(2-cyano-phenyl- | H | methoxy | 2,6-diethyl- |
| | | amino- | | | phenyl |
| 75 | N | N-(2-aminomethyl- | H | methoxy | 2,6-diethyl- |
| | | phenyl)-amino- | | | phenyl |
| 76 | N | N-ethyl-N-(2,3-dimethyl- | H | methoxy | 2,6- |
| | | phenyl)-amino- | | | dimethyl- |
| | | | | | phenyl |
| 77 | N | (S)-N-methyl-N-(S)- | H | methoxy | 2,6-diethyl- |
| | | (1,2,3,4-tetrahydro- | | | phenyl |
| | | naphth-1-yl)-amino- | | | |
| 78 | N | N-ethyl-N-(naphth-1-yl)- | H | methoxy | 1-naphthyl |
| | | amino- | | | |
| 79 | N | N-(methylcarbonyl)-N- | H | methoxy | 2,6-diethyl- |
| | | (naphth-1-yl)-amino- | | | phenyl |
| 80 | N | N-methyl-N-(2-methyl- | H | methoxy | 2,6-diethyl- |
| | | 4-methoxy-phenyl)- | | | phenyl |
| | | amino- | | | |
| 81 | N | N-(3-methoxy-phenyl)- | H | methoxy | 2,6-diethyl- |
| | | amino- | | | phenyl |
| 82 | N | N-methyl-N-phenyl- | H | methoxy | 2,6-diethyl- |
| | | amino- | | | phenyl |
| 83 | N | (R)-N-methyl-N-(R)- | H | methoxy | 2,6-diethyl- |
| | | (1,2,3,4-tetrahydro- | | | phenyl |
| | | naphth-1-yl)-amino- | | | |

Some of the compounds of the present invention may contain chiral centers and such compounds may exist in the form of stereoisomers (i.e. enantiomers). The present invention includes all such stereoisomers and any mixtures thereof including racemic mixtures. Racemic mixtures of the stereoisomers as well as the substantially pure stereoisomers are within the scope of the invention. The term “substantially pure,” as used herein, refers to at least about 90 mole %, more preferably at least about 95 mole %, and most preferably at least about 98 mole % of the desired stereoisomer is present relative to other possible stereoisomers. Preferred enantiomers may be isolated from racemic mixtures by any method known to those skilled in the art, including high performance liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron, 33:2725 (1977); Eliel, E. L. Stereochemistry of Carbon Compounds, (McGraw-Hill, NY, 1962); Wilen, S. H. Tables of Resolving Agents and Optical Resolutions, p. 268 (E. L. Eliel, Ed., University of Notre Dame Press, Notre Dame, Ind. 1972).

Within the present invention, the compounds of formula (I) may be prepared in the form of pharmaceutically acceptable salts. As used herein, the term “pharmaceutically acceptable salts” refers to salts prepared from pharmaceutically acceptable non-toxic acids, including inorganic salts and organic salts. Suitable salts include inorganic and organic acids such as acetic, benzenesulfonic, benzoic, camphorsulfonic, citric, ethenesulfonic, fumaric, gluconic, glutamic, hydrobromic, hydrochloric, isethionic, lactic, malic, maleic, mandelic, methanesulfonic, mucic, nitric, pamoic, pantothenic, phosphoric, succinic, sulfuric, tartaric acid, p-toluenesulfonic and the like. Particularly preferred are hydrochloric, hydrobromic, phosphoric, and sulfuric acids, and most preferably is the hydrochloride salt.

The present invention includes prodrugs of the compounds of formula (I). “Prodrug,” as used herein, means a compound which is convertible in vivo by metabolic means (e.g. by hydrolysis) to a compound of formula (I). Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs,” Textbook of Drug Design and Development, Chapter 5, 113-191 (1991), Bundgaard, et al., Journal of Drug Deliver Reviews, 1992, 8:1-38, Bundgaard, J. of Pharmaceutical Sciences, 1988, 77:285 et seq.; and Higuchi and Stella (eds.) Prodrugs as Novel Drug Delivery Systems, American Chemical Society (1975).

Further, the compounds of formula (I) may exist in unsolvated as well as in solvated forms with pharmaceutically acceptable solvents such as water, ethanol, and the like. In general, the solvated forms are considered equivalent to the unsolvated forms for the purpose of the present invention.

As more extensively provided in this written description, terms such as “reacting” and “reacted” are used herein in reference to a chemical entity that is any one of: (a) the actually recited form of such chemical entity, and (b) any of the forms of such chemical entity in the medium in which the compound is being considered when named.

One skilled in the art will recognize that, where not otherwise specified, the reaction step(s) is performed under suitable conditions, according to known methods, to provide the desired product. One skilled in the art will further recognize that, in the specification and claims as presented herein, wherein a reagent or reagent class/type (e.g. base, solvent, etc.) is recited in more than one step of a process, the individual reagents are independently selected for each reaction step and may be the same of different from each other. For example wherein two steps of a process recite an organic or inorganic base as a reagent, the organic or inorganic base selected for the first step may be the same or different than the organic or inorganic base of the second step. Further, one skilled in the art will recognize that wherein a reaction step of the present invention may be carried out in a variety of solvents or solvent systems, said reaction step may also be carried out in a mixture of the suitable solvents or solvent systems. One skilled in the art will further recognize that wherein two consecutive reaction or process steps are run without isolation of the intermediate product (i.e. the product of the first of the two consecutive reaction or process steps), then the first and second reaction or process steps may be run in the same solvent or solvent system; or alternatively may be run in different solvents or solvent systems following solvent exchange, which may be completed according to known methods.

As used herein, unless otherwise noted, the term “aprotic solvent” shall mean any solvent that does not yield a proton. Suitable examples include, but are not limited to DMF, 1,4-dioxane, THF, acetonitrile, pyridine, dichloroethane, dichloromethane, MTBE, toluene, acetone, and the like.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, and the like.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any range therein.

Examples of suitable solvents, bases, reaction temperatures, and other reaction parameters and components are provided in the detailed descriptions which follows herein. One skilled in the art will recognize that the listing of said examples is not intended, and should not be construed, as limiting in any way the invention set forth in the claims which follow thereafter.

The compounds of the present invention may be prepared in a number of ways well known to those skilled in the art. The compounds can be synthesized, for example, by the methods described below, or variations thereon as appreciated by the skilled artisan. All processes disclosed in association with the present invention are contemplated to be practiced on any scale, including milligram, gram, multigram, kilogram, multikilogram or commercial industrial scale.

As will be readily understood, functional groups present may contain protecting groups during the course of synthesis. Protecting groups are known per se as chemical functional groups that can be selectively appended to and removed from functionalities, such as hydroxyl groups and carboxyl groups. These groups are present in a chemical compound to render such functionality inert to chemical reaction conditions to which the compound is exposed. Any of a variety of protecting groups may be employed with the present invention. Protecting groups that may be employed in accordance with the present invention may be described in Greene, T. W. and Wuts, P. G. M., Protective Groups in Organic Synthesis 2d. Ed., Wiley & Sons, 1991.

Compounds of the present invention are suitably prepared in accordance with the following general description and specific examples. Variables used are as defined for formula (I), unless otherwise noted. The reagents used in the preparation of the compounds of this invention can be either commercially obtained or can be prepared by standard procedures described in the literature. In accordance with this invention, compounds of formula (I) may be produced by the following reaction schemes (Schemes A to G).

The compounds of this invention contain chiral centers, providing for various stereoisomeric forms such as diastereomeric mixtures, enantiomeric mixtures as well as optical isomers. The individual optical isomers can be prepared directly through asymmetric and/or stereospecific synthesis or by conventional chiral separation of optical isomers from the enantiomeric mixture.

Representative compounds of the present invention can be synthesized in accordance with the general synthetic methods described below and illustrated in the schemes that follow. Unless otherwise indicated, the substituents (e.g., X, Y, Z, R¹, R², R³, R⁴, R⁵, R⁶, R⁷, R⁸, and Q) in the following schemes shall have the meanings as those described above. Since the schemes are an illustration, the invention should not be construed as being limited by the chemical reactions and conditions expressed. The preparation of the various starting materials used in the schemes is well within the skill of persons versed in the art.

The following schemes describe general synthetic methods whereby intermediates and target compounds of the present invention may be prepared. Additional representative compounds and stereoisomers, racemic mixtures, diastereomers, and enantiomers thereof can be synthesized using the intermediates prepared in accordance to the general schemes and other materials, compounds and reagents known to those skilled in the art. All such compounds, stereoisomers, racemic mixtures, diastereomers, and enantiomers thereof are intended to be encompassed within the scope of the present invention.

Compounds of formula (I) may be prepared according to the processes as described in more detail below. Compounds of formula (I) wherein Q is N, X is selected from the group consisting of NR¹R², OR³ and SR³, R⁵ is hydrogen, Y is selected from the group consisting of OR⁷ and SR⁷ and Z is as herein defined, may be prepared according to the process outlined in Scheme 1, below.

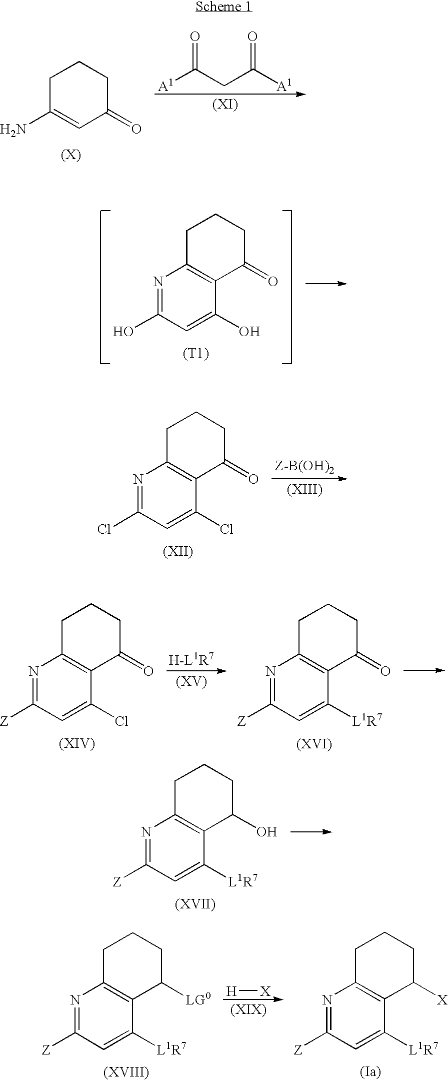

Accordingly, a suitably substituted compound of formula (X), a known compound or compound prepared by known methods, is reacted with a suitably selected diester or acid chloride (wherein A¹ is Cl) of malonic acid, a compound of formula (XI) wherein A¹ is a suitably selected leaving group such as Cl, 2,4,6-trichlorophenyloxy, 2,4,6-trifluorophenyloxy, and the like; in an organic solvent such as bromobenzene, xylenes, and the like; at an elevated temperature in the range of from about 100° C. to about 200° C., for example at about solvent reflux temperature; to yield the corresponding compound of formula (T1), which is preferably not isolated.

The compound of formula (T1) is reacted with a suitably selected chlorinating agent, such as phosphorous oxychloride, thionyl chloride, phenylphosphonic chloride, and the like; neat or in a suitably selected organic solvent; to yield the corresponding compound of formula (XII).

The compound of formula (XII) is reacted with a suitably substituted compound of formula (XIII), a known compound or compound prepared by known methods, in the presence of a suitably selected coupling agent such as Pd(PPh₃)₄, Pd₂(dba)₃, Pd(PPh₃)₂Cl₂, and the like; in the presence of a base such as sodium carbonate, potassium carbonate, cesium carbonate, and the like; in a mixture of water and an organic solvent such as a mixture of water and acetonitrile, water and THF, water and toluene, and the like; to yield the corresponding compound of formula (XIV).

The compound of formula (XIV) is reacted with a suitably substituted compound of formula (XV), wherein L¹ is O or S, a known compound or compound prepared by known methods, in the presence of a base such as NaH, potassium hexamethyldisilazide, sodium metal, and the like; neat or in an aprotic organic solvent such as THF, acetonitrile, and the like; to yield the corresponding compound of formula (XVI).

The compound of formula (XVI) is reacted with a suitably selected reducing agent such as NaBH₄, lithium borohydride, and the like; in an organic solvent such as methanol, ethanol, and the like; or with a suitably selected reducing agent such as LAH, and the like; in an aprotic organic solvent such as THF, diethyl ether, and the like; to yield the corresponding compound of formula (XVII).

The compound of formula (XVII) is reacted with a suitably selected halogenating agent such as sulfonyl chloride, thionyl chloride, POCl₃, POBr₃, a mixture of triphenylphosphine and bromine, and the like; neat or in an aprotic organic solvent such as DCM, DCE, and the like; to yield the corresponding compound of formula (XVIII), wherein LG⁰ is the corresponding leaving group Cl, Br, respectively. Alternatively, the compound of formula (XVII) is reacted with a suitably selected activating agent such as mesyl chloride, triflic anhydride, and the like, in an aprotic organic solvent such as DCM, DCE, and the like; to yield the corresponding compound of formula (XVIII), wherein LG⁰ is the corresponding leaving group mesyl, trifluoromethane sulfonyl, respectively.

The compound of formula (XVIII) is reacted with a suitably substituted compound of formula (XIX)), a known compound or compound prepared by known methods, in the presence of a base such as potassium carbonate, sodium carbonate, cesium carbonate, and the like; or wherein the compound of formula (XIX) is a base, in the presence of about 2 equivalents or more of the compound of formula (XIX); in an aprotic organic solvent such as acetonitrile, THF, and the like; to yield the corresponding compound of formula (Ia).

One skilled in the art will recognize that when the compound of formula (XVIII) is reacted with ammonia, the resulting compound of formula (Ia), wherein X is NH₂ may be further reacted, according to known methods, to further substitute with terminal amine groups.

One skilled in the art will further recognize that the reaction steps outlined in Scheme 1 above, may be applied in alternative sequences, to yield the corresponding compounds of formula (Ia). As an example, Schemes 2 and 3 below show synthesis of compounds of formula (Ia), wherein the sequence of the reaction steps are alternative to those described in Scheme 1 above.

Compounds of formula (I) wherein Q is N, X is selected from the group consisting of NR¹R², OR³ and SR³, R⁵ is hydrogen, Y is selected from the group consisting of OR⁷ and SR⁷, and Z is as herein defined, may alternatively be prepared according to the process outlined in Scheme 2, below.

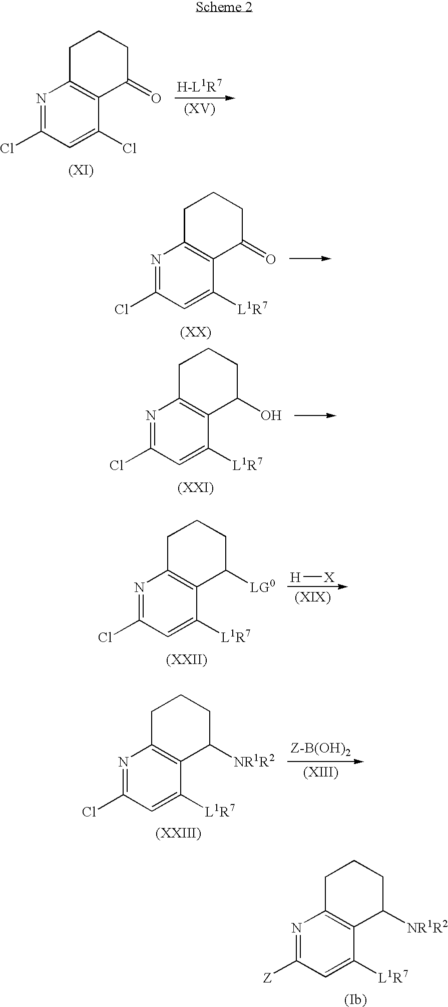

Accordingly, a suitably substituted compound of formula (XI) is reacted with a suitably substituted compound of formula (XV), a known compound or compound prepared by known methods, wherein L¹ is O or S, a known compound or compound prepared by known methods, in the presence of a base such as NaH, potassium hexamethyldisilazide, sodium metal, and the like; neat or in an aprotic organic solvent such as THF, acetonitrile, and the like; to yield the corresponding compound of formula (XX).

The compound of formula (XX) is reacted with a suitably selected reducing agent such as NaBH₄, lithium borohydride, and the like; in an organic solvent such as methanol, ethanol, and the like; or with a suitably selected reducing agent such as LAH, and the like; in an aprotic organic solvent such as THF, diethyl ether, and the like; to yield the corresponding compound of formula (XXI).

The compound of formula (XXI) is reacted with a suitably selected halogenating agent such as sulfonyl chloride, thionyl chloride, POCl₃, POBr₃, a mixture of triphenylphosphine and bromine, and the like; neat or in an aprotic organic solvent such as DCM, DCE, and the like; to yield the corresponding compound of formula (XXII), wherein LG⁰ is the corresponding leaving group Cl, Br, respectively. Alternatively, the compound of formula (XXI) is reacted with a suitably selected activating agent such as mesyl chloride, triflic anhydride, and the like, in an aprotic organic solvent such as DCM, DCE, and the like; to yield the corresponding compound of formula (XXII), wherein LG⁰ is the corresponding leaving group mesyl, trifluoromethane sulfonyl, respectively.

The compound of formula (XXII) is reacted with a suitably substituted compound of formula (XIX), a known compound or compound prepared by known methods, in the presence of a base such as potassium carbonate, sodium carbonate, cesium carbonate, and the like; or wherein the compound of formula (XIX) is a base, in the presence of about 2 equivalents or more of the compound of formula (XIX); in an aprotic organic solvent such as acetonitrile, THF, and the like; to yield the corresponding compound of formula (XXIII).

The compound of formula (XXIII) is reacted with a suitably substituted compound of formula (XIII), a known compound or compound prepared by known methods, in the presence of a suitably selected coupling agent such as Pd(PPh₃)₄, Pd₂(dba)₃, Pd(PPh₃)₂Cl₂, and the like; in the presence of a base such as sodium carbonate, potassium carbonate, cesium carbonate, and the like; in a mixture of water and an organic solvent such as a mixture of water acetonitrile, water and toluene, water and THF, and the like; to yield the corresponding compound of formula (Ib).

Compounds of formula (I) wherein Q is N, X is selected from the group consisting of NR¹R², OR³ and SR³, R⁵ is hydrogen, Y is selected from the group consisting of OR⁷ and SR⁷ and Z is as herein defined, may alternatively be prepared according to the process outlined in Scheme 3, below.

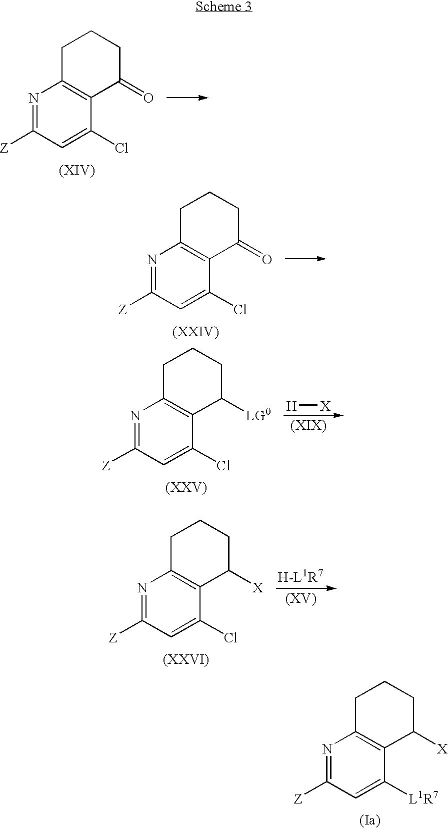

Accordingly, a suitably substituted compound of formula (XIV) is reacted with a suitably selected reducing agent such as NaBH₄, lithium borohydride, and the like; in an organic solvent such as methanol, ethanol, and the like; or with a suitably selected reducing agent such as LAH, and the like; in an aprotic organic solvent such as THF, diethyl ether, and the like; to yield the corresponding compound of formula (XXIV).

The compound (XXIV) is reacted with a suitably selected halogenating agent such as sulfonyl chloride, thionyl chloride, POCl₃, POBr₃, a mixture of triphenylphosphine and bromine, and the like; neat or in an aprotic organic solvent such as DCM, DCE, and the like; to yield the corresponding compound of formula (XXV), wherein LG⁰ is the corresponding leaving group Cl, Br, respectively. Alternatively, the compound of formula (XXIV) is reacted with a suitably selected activating agent such as mesyl chloride, triflic anhydride, and the like, in an aprotic organic solvent such as DCM, DCE, and the like; to yield the corresponding compound of formula (XXV), wherein LG⁰ is the corresponding leaving group mesyl, trifluoromethane sulfonyl, respectively.

The compound of formula (XXV) is reacted with a suitably substituted compound of formula (XIX), a known compound or compound prepared by known methods, in the presence of a base such as potassium carbonate, sodium carbonate, cesium carbonate, and the like; or wherein the compound of formula (XIX) is a base, in the presence of about 2 equivalents or more of the compound of formula (XIX); in an aprotic organic solvent such as acetonitrile, THF, and the like; to yield the corresponding compound of formula (XXVI).

The compound of formula (XXVI) is reacted with a suitably substituted compound of formula (XV), a known compound or compound prepared by known methods, wherein L¹ is O or S, a known compound or compound prepared by known methods, in the presence of a base such as NaH, potassium hexamethyldisilazide, sodium metal, and the like; neat or in an aprotic organic solvent such as THF, acetonitrile, and the like; to yield the corresponding compound of formula (Ia).

Compounds of formula (I) wherein Q is N, X is selected from the group consisting of NR¹R², OR³ and SR³, R⁵ is hydrogen, Y is selected from the group consisting of R⁸, and Z is as herein defined, may alternatively be prepared according to the process outlined in Scheme 4, below.

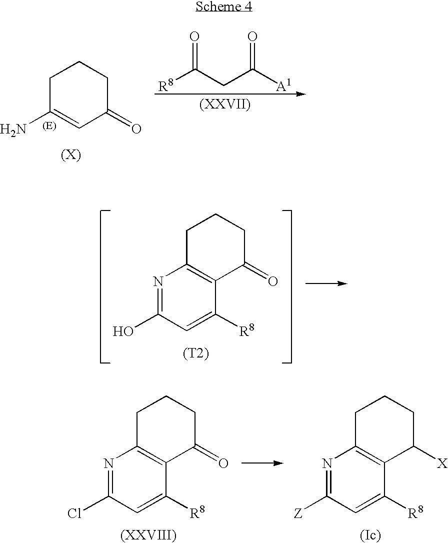

Accordingly, a suitably substituted compound of formula (X), a known compound or compound prepared by known methods, is reacted with a suitably substituted compound of formula (XXVIII), a known compound or compound prepared by known methods, wherein R⁸ is as herein defined and wherein A¹ is a suitably selected leaving group such as Cl, 2,4,6-trichlorophenyloxy, 2,4,6-trifluorophenyloxy, and the like; in an organic solvent such as bromobenzene, xylenes, and the like; at an elevated temperature in the range of from about 100° C. to about 200° C., for example at about solvent reflux temperature; to yield the corresponding compound of formula (T2), which is preferably not isolated.

The compound of formula (T2) is reacted with a suitably selected chlorinating agent, such as phosphorous oxychloride, thionyl chloride, phenylphosphonic dichloride, and the like; neat or in a suitably selected organic solvent; to yield the corresponding compound of formula (XXVIII).

The compound of formula (XXVIII) is then further reacted to yield the corresponding compound of formula (Ic). For example, the compound of formula (XXVIII) may be substituted for the compound of formula (XX) in Scheme 2, and reacted as described, to yield the compound of formula (Ic).

Compounds of formula (I) wherein Q is N, X is selected from the group consisting of NR¹R², OR³ and SR³, R⁵ is as herein defined, Y is selected from the group consisting of OR⁷, SR⁷ and R⁸, and Z is as herein defined, may be prepared according to the process outlined in Scheme 5, below.

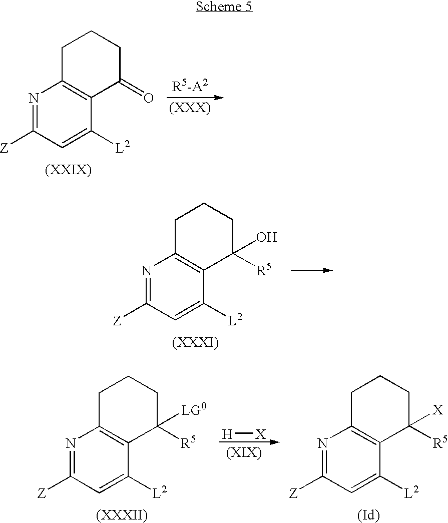

Accordingly, a suitably substituted compound of formula (XXIX), a known compound or compound prepared as described herein, wherein L² is selected from the group consisting of OR⁷, SR⁷ and R⁸, is reacted with a suitably substituted compound of formula (XXX), wherein A² is MgBr, MgCl or Li, a known compound or compound prepared by known methods; in an aprotic organic solvent such as THF, 1,4-dioxane, MTBE, and the like; followed by treatment with a suitably selected proton source such as water, aqueous ammonium chloride, and the like; to yield the corresponding compound of formula (XXXI).

The compound of formula (XXXI) is reacted with a suitably selected halogenating agent such as sulfonyl chloride, thionyl chloride, POCl₃, POBr₃, a mixture of triphenylphosphine and bromine, and the like; neat or in an aprotic organic solvent such as DCM, DCE, and the like; to yield the corresponding compound of formula (XXXII), wherein LG⁰ is the corresponding leaving group Cl, Br, respectively. Alternatively, the compound of formula (XXXI) is reacted with a suitably selected activating agent such as mesyl chloride, triflic anhydride, and the like, in an aprotic organic solvent such as DCM, DCE, and the like; to yield the corresponding compound of formula (XXXII), wherein LG⁰ is the corresponding leaving group mesyl, trifluoromethane sulfonyl, respectively.

The compound of formula (XXXII) is reacted with a suitably substituted compound of formula (XIX), a known compound or compound prepared by known methods, in the presence of a base such as potassium carbonate, sodium carbonate, cesium carbonate, and the like; or wherein the compound of formula (XIX) is a base, in the presence of about 2 equivalents or more of the compound of formula (XIX); in an aprotic organic solvent such as acetonitrile, THF, and the like; to yield the corresponding compound of formula (Id).

Compounds of formula (I) wherein Q is N, X is selected from the group consisting of NR¹R², OR³ and SR³, R⁵ is hydrogen, Y is hydrogen, and Z is as herein defined, may be prepared according to the process outlined in Scheme 6, below.

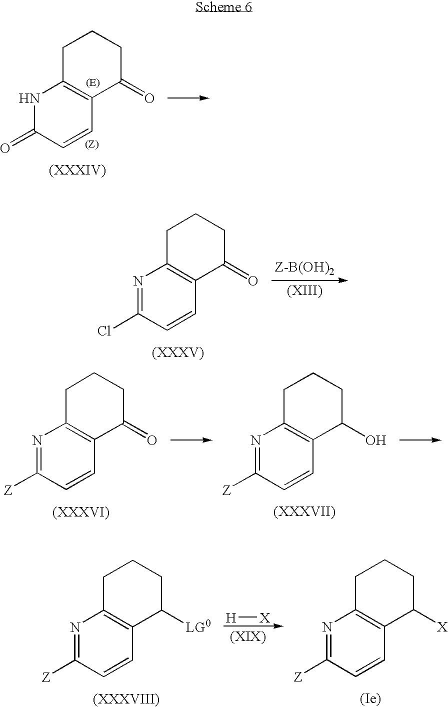

Accordingly, a suitably substituted compound of formula (XXXIV), a known compound or compound prepared by known methods, is reacted with a suitably selected chlorinating agent, such as phosphorous oxychloride, thionyl chloride, phenylphosphonic dichloride, and the like; neat or in a suitably selected organic solvent; to yield the corresponding compound of formula (XXXV).

The compound of formula (XXXV) is reacted with a suitably substituted compound of formula (XIII), a known compound or compound prepared by known methods, is reacted with a suitably selected halogenating agent such as sulfonyl chloride, thionyl chloride, POCl₃, POBr₃, a mixture of triphenylphosphine and bromine, and the like; neat or in an aprotic organic solvent such as DCM, DCE, and the like; to yield the corresponding compound of formula (XXXVI), wherein LG⁰ is the corresponding leaving group Cl, Br. Alternatively, the compound of formula (XXXV) is reacted with a suitably selected activating agent such as mesyl chloride, triflic anhydride, and the like, in an aprotic organic solvent such as DCM, DCE, and the like; to yield the corresponding compound of formula (XXXVI), wherein LG⁰ is the corresponding leaving group mesyl, trifluoromethane sulfonyl.

The compound of formula (XXXVI) is reacted with a suitably selected reducing agent such as NaBH₄, lithium borohydride, and the like; in an organic solvent such as methanol, ethanol, and the like; or with a suitably selected reducing agent such as LAH, and the like; in an aprotic organic solvent such as THF, diethyl ether, and the like; to yield the corresponding compound of formula (XXXVII).

The compound of formula (XXXVII) is reacted with a suitably selected halogenating agent such as sulfonyl chloride, thionyl chloride, POCl₃, POBr₃, a mixture of triphenylphosphine and bromine, and the like; neat or in an aprotic organic solvent such as DCM, DCE, and the like; to yield the corresponding compound of formula (XXXVIII), wherein LG⁰ is the corresponding leaving group Cl, Br, respectively. Alternatively, the compound of formula (XXXVII) is reacted with a suitably selected activating agent such as mesyl chloride, triflic anhydride, and the like, in an aprotic organic solvent such as DCM, DCE, and the like; to yield the corresponding compound of formula (XXXVIII), wherein LG⁰ is the corresponding leaving group mesyl, trifluoromethane sulfonyl, respectively.

The compound of formula (XXXVIII) is reacted with a suitably substituted compound of formula (XIX)), a known compound or compound prepared by known methods, in the presence of a base such as potassium carbonate, sodium carbonate, cesium carbonate, and the like; or wherein the compound of formula (XIX) is a base, in the presence of about 2 equivalents or more of the compound of formula (XIX); in an aprotic organic solvent such as acetonitrile, THF, and the like; to yield the corresponding compound of formula (Ie).

One skilled in the art will recognize that compounds of formula (I) wherein Q is N, X is selected from the group consisting of NR¹R², OR³ and SR³, R⁵ is other than hydrogen, Y is hydrogen, and Z is as herein defined may be similarly prepared by substituting the compound of formula (XXXVI) for the compound of formula (XXIX), in Scheme 5, as described above.

Compounds of formula (I) wherein Q is N+O— may be prepared from the corresponding compound of formula (I) wherein Q is N as outlined in Scheme 7 below.

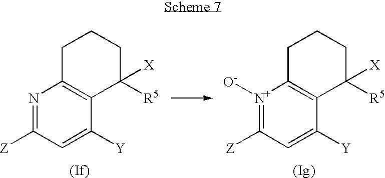

Accordingly, a suitably substituted compound of formula (If), prepared as described above, is reacted with a suitably selected oxidizing agent such as mCPBA, and the like; in a suitably selected organic solvent such as THF, DCM, DCE, and the like; or with a suitably selected oxidizing agent such as hydrogen peroxide, and the like; in a suitable selected solvent such as water; to yield the corresponding compound of formula (Ig).

Compounds of formula (I) wherein X is NR¹R² and wherein R² is—C(O)—R⁸ may be prepared from the corresponding compound of formula (I) wherein X is NHR¹, as outlined in Scheme 8, below.

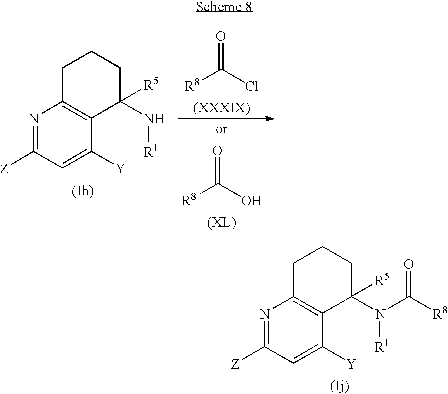

Accordingly, a suitably substituted compound of formula (Ih), prepared as described herein, is reacted with a suitably substituted compound of formula (XXXIX), a known compound or compound prepared by known methods, in the presence of a base such as TEA, DIPEA, pyridine, and the like, in an organic solvent such as THF, toluene, DCM, and the like; to yield the corresponding compound of formula (Ij).

Alternatively, a suitably substituted compound of formula (Ih), prepared as described herein, is reacted with a suitably substituted compound of formula (XL), a known compound or compound prepared by known methods, in the presence of suitably selected peptide coupling agent such as EDC in combination with HOBT, DCC in combination with HOBT, and the like; preferably in the presence of a suitably selected base such as TEA, DIPEA, pyridine, and the like, in an organic solvent such as DCM, DCE, acetonitrile, THF, and the like; to yield the corresponding compound of formula (Ij).

Compounds of formula (I) wherein Y is ethyl may alternatively be prepared according to the process outlined in Scheme 9, below.

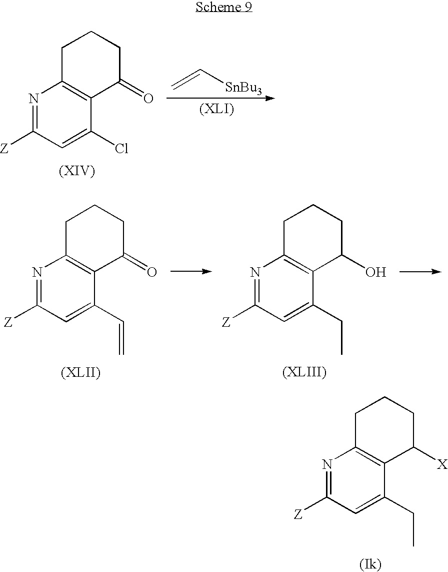

Accordingly, a suitably substituted compound of formula (XIV), prepared as described herein, is reacted with a suitably substituted compound of formula (XLI), a known compound or compound prepared by known methods, in the presence of a suitably selected coupling agent such as Pd(PPh₃)₄, Pd₂(dba), Pd(PPh₃)₂Cl₂, and the like, in an organic solvent such as 1,4-dioxane, THF, DMF, and the like; to yield the corresponding compound of formula (XLII).

The compound of formula (XLII) is reacted with a suitably selected reducing agent such as NaBH₄, lithium borohydride, and the like; in an organic solvent such as methanol, ethanol, and the like; or with a suitably selected reducing agent such as LAH, and the like; in an aprotic organic solvent such as THF, diethyl ether, and the like; to yield the corresponding compound of formula (XLIII).

The compound of formula (XLIII) is then further reacted according to the processes described herein, to yield the corresponding compound of formula (Ik).

One skilled in the art will further recognize that compounds of formula (I) wherein Y is halogen (i.e. chloro, fluoro, iodo or bromo) may be prepared as described in the Schemes above, with selection and substitution of appropriate intermediates. For example, the intermediate compounds of formula (XXVI), prepared as described in Scheme 3 above, corresponding to compounds of formula (I) wherein R⁵ is hydrogen and Y is halogen.

In other embodiments, the invention is directed to pharmaceutical compositions, comprising: (a) at least a compound of formula (I), or pharmaceutically acceptable salt thereof; and (b) at least one pharmaceutically acceptable carrier. Generally, the compound of formula (I), or a pharmaceutically acceptable salt thereof, will be present at a level of from about 0.1%, by weight, to about 90% by weight, based on the total weight of the pharmaceutical composition, based on the total weight of the pharmaceutical composition. Preferably, the compound of formula (I), or a pharmaceutically acceptable salt thereof, will be present at a level of at least about 1%, by weight, based on the total weight of the pharmaceutical composition. More preferably, the compound of formula (I), or a pharmaceutically acceptable salt thereof, will be present at a level of at least about 5%, by weight, based on the total weight of the pharmaceutical composition. Even more preferably, the compound of formula (I) or a pharmaceutically acceptable salt thereof will be present at a level of at least about 10%, by weight, based on the total weight of the pharmaceutical composition. Yet even more preferably, the compound of formula (I), or a pharmaceutically acceptable salt thereof, will be present at a level of at least about 25%, by weight, based on the total weight of the pharmaceutical composition.

Such compositions are prepared in accordance with acceptable pharmaceutical procedures, such as described in Remington's Pharmaceutical Sciences, 17th edition, ed. Alfonoso R. Gennaro, Mack Publishing Company, Easton, Pa. (1985). Pharmaceutically acceptable carriers are those that are compatible with the other ingredients in the formulation and biologically acceptable.

The compounds of this invention may be administered orally or parenterally, neat or in combination with conventional pharmaceutical carriers. Applicable solid carriers can include one or more substances that may also act as flavoring agents, lubricants, solubilizers, suspending agents, fillers, glidants, compression aids, binders or tablet-disintegrating agents or an encapsulating material. In powders, the carrier is a finely divided solid that is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers may be used in preparing solutions, suspensions, emulsions, syrups, and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also be an oily ester such as ethyl oleate and isopropyl myristate. Sterile liquid carriers are used in sterile liquid form compositions for parenteral administration.

Liquid pharmaceutical compositions, which are sterile solutions or suspensions, can be administered by, for example, intramuscular, intraperitoneal or subcutaneous injection. Sterile solutions can also be administered intravenously. Oral administration may be either liquid or solid composition form.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example packeted powders, vials, ampoules, prefilled syringes or sachets containing liquids. The unit dosage form can be, for example, a capsule or tablet itself, or it can be the appropriate number of any such compositions in package form.

In another embodiment of the present invention, the compounds useful in the present invention may be administered to a patient with one or more other pharmaceutical active agents such as those agents being used to treat any other medical condition present in the patient. Examples of such pharmaceutical active agents include anti-inflammatory agents, immunosuppressive agents, agents directed towards suppression of the complement response, pain relieving agents, anti-angiogenic agents, anti-neoplastic agents, anti-diabetic agents, anti-infective agents, or gastrointestinal agents, or combinations thereof.

The one or more other pharmaceutical active agents may be administered in a therapeutically effective amount simultaneously (such as individually at the same time, or together in a pharmaceutical composition), and/or successively with one or more compounds of the present invention.

The term “combination therapy” refers to the administration of two or more therapeutic agents or compounds to treat a therapeutic condition or disorder described in the present disclosure, for example, immune and inflammatory diseases and conditions, including sepsis, rheumatoid arthritis, psoriasis, inflammatory bowel disease, immune complex diseases, systemic lupus erythematosus, lupus nephritis, Alzheimer's disease, ischemia/reperfusion injury, multiple sclerosis, myasthenia gravis, glomerulonephritis, chronic graft rejection, gingivitis, asthma, dermatitis, Guillain-Barre syndrome, myocardial infarct, pancreatitis, cystic fibrosis, atherosclerosis, fibrosis, allergies, diabetes type I, and combinations thereof. Such administration includes use of each type of therapeutic agent in a concurrent manner. In either case, the treatment regimen will provide beneficial effects of the drug combination in treating the conditions or disorders described herein.

The route of administration may be any route, which effectively transports the active compound of formula (I), or a pharmaceutically acceptable salt thereof, to the appropriate or desired site of action, such as oral, nasal, pulmonary, transdermal, such as passive or iontophoretic delivery, or parenteral, e.g. rectal, depot, subcutaneous, intravenous, intraurethral, intramuscular, intranasal, ophthalmic solution or an ointment. Furthermore, the administration of compound of formula (I), or pharmaceutically acceptable salt thereof, with other active ingredients may be concurrent or simultaneous.

Preferably, the compounds of the present invention are pharmaceutically active as C5a receptor antagonists.

It is believed that the present invention described presents compounds for use in the field of treatment, alleviation, inhibition, and/or prevention of conditions related to C5a receptor modulation, including, inter alia, immune and inflammatory diseases and conditions, including sepsis, rheumatoid arthritis, psoriasis, inflammatory bowel disease, immune complex diseases, systemic lupus erythematosus, lupus nephritis, Alzheimer's disease, ischemia/reperfusion injury, multiple sclerosis, myasthenia gravis, glomerulonephritis, chronic graft rejection, gingivitis, asthma, dermatitis, Guillain-Barre syndrome, myocardial infarct, pancreatitis, cystic fibrosis, atherosclerosis, fibrosis, allergies, diabetes type I, and combinations thereof. Preferably compounds of the present invention are for use in the field of treatment sepsis, rheumatoid arthritis, psoriasis, inflammatory bowel disease, immune complex diseases, systemic lupus erythematosus, lupus nephritis, Alzheimer's disease, ischemia/reperfusion injury, and combinations thereof.

Accordingly, in another embodiment, the invention is directed to methods of treating an immune or inflammatory disease or condition in a patient in need thereof, comprising the step of: administering to said patient an effective amount of a compound of formula (I) or a pharmaceutically acceptable salt thereof. The immune and inflammatory diseases and conditions include sepsis, rheumatoid arthritis, psoriasis, inflammatory bowel disease, immune complex diseases, systemic lupus erythematosus, lupus nephritis, Alzheimer's disease, ischemia/reperfusion injury, multiple sclerosis, myasthenia gravis, glomerulonephritis, chronic graft rejection, gingivitis, asthma, dermatitis, Guillain-Barre syndrome, myocardial infarct, pancreatitis, cystic fibrosis, atherosclerosis, fibrosis, allergies, diabetes type I, and combinations thereof. Preferably the immune or inflammatory disease or condition is sepsis, rheumatoid arthritis, psoriasis, inflammatory bowel disease, immune complex diseases, systemic lupus erythematosus, Alzheimer's disease, and/or ischemia/reperfusion injury.

The present invention is further defined in the following Examples, in which all parts and percentages are by weight and degrees are Celsius, unless otherwise stated. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various usages and conditions.

Specific compounds which are representative of this invention were prepared as per the following examples and reaction sequences. The examples and the diagrams depicting the reaction sequences are offered by way of illustration, to aid in the understanding of the invention and should not be construed to limit in any way the invention set form in the claims which follow thereafter. The instant compounds may also be used as intermediates in subsequent examples to produce additional compounds of the present invention. No attempt has been made to optimize the yields obtained in any of the reactions. One skilled in the art would know how to increase such yields through routine variations in reaction times, temperatures, solvents, and/or reagents.

The following Examples are set forth to aid in the understanding of the invention, and are not intended and should not be construed to limit in any way the invention set forth in the claims which follow thereafter.

In the Examples which follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

### Example 1

### (±)-[2-(2,6-Diethyl-phenyl)-4-methoxy-5,6,7,8-tetrahydro-quinolin-5-yl]-ethyl-naphthlen-1-yl-amine (Compound #12)

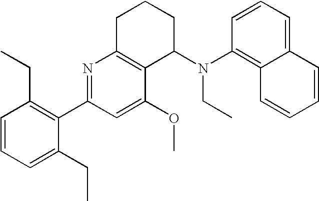

A mixture of 3-amino-cyclohex-2-enone (12.5 g, 113 mmol, 1 equiv) and malonic acid bis-(2,4,6-trichlorophenyl) ester (52.1 g, 113 mmol, 1 equiv) in bromobenzene (120 mL) was heated in a 155° C. oil bath for 30 minutes. The reaction mixture was allowed to cool and was poured into a mixture of EtOAc (240 mL) and Et₂O (240 mL). The precipitate was collected by vacuum filtration and the collected solids were washed with EtOAc and were air-dried. The solid was treated with phosphorus oxychloride (86 mL, 939 mmol) and the resultant mixture was heated in a 95° C. oil bath for 4 hours. The reaction mixture was allowed to cool and was then concentrated. The residue was poured into ice (100 mL) and the mixture was neutralized to pH 7 by addition of solid Na₂CO₃ (gas evolution). The mixture was extracted with CH₂Cl₂ (3×100 mL), and the organic extracts were dried (Na₂SO₄), filtered, and concentrated. The residue was purified by flash column chromatography (SiO₂, 0-15% EtOAc in hexanes), to yield 2,4-dichloro-7,8-dihydro-6H-quinolin-5-one (4.81 g, 30%) as a yellow solid.

¹H NMR (400 MHz, CDCl₃) δ 7.34 (s, 1H), 3.16 (t, 2H), 2.72 (t, 2H), 2.16 (t, 2H); MS m/z (MH⁺) 216.0.

To a mixture of 2,4-dichloro-7,8-dihydro-6H-quinolin-5-one (7.26 g, 33.6 mmol, 1 equiv), 2,6-diethylphenylboronic acid (6.28 g, 35.3 mmol, 1.05 equiv) and Pd(PPh₃)₄ (1.94 g, 1.68 mmol, 0.05 equiv) was added 2 M aqueous Na₂CO₃ (67 mL) and toluene (67 mL). The resulting mixture was heated at reflux under nitrogen for 31 hours. The mixture was partitioned between EtOAc (100 mL) and water (100 mL). The separated aqueous phase was extracted with two additional 100-mL portions of EtOAc. The organic extracts were dried (Na₂SO₄) and filtered, and the filtrate was concentrated. Flash column chromatography (SiO₂, first column: 5-15% EtOAc in hexanes, second column: 0-5% Et₂O in CH₂Cl₂) yielded 4-chloro-2-(2,6-diethyl-phenyl)-7,8-dihydro-6H-quinolin-5-one (6.96 g, 66%).

Analysis of a representative sample of this product indicated that the isolated material was contaminated by a byproduct in which both the 2- and 4-positions of the 5,6,7,8-tetrahydroquinoline group were arylated (estimated at 11% by integration of LCMS UV chromatograph). ¹H NMR (300 MHz, CDCl₃) δ 7.26-7.34 (m, 2H), 7.15 (d, 2H), 3.23 (t, 2H), 2.78 (t, 2H), 2.30 (q, 4H), 2.17-2.27 (m, 2H), 1.07 (t; 6H); MS m/z (MH⁺) 314.1.

A solution of sodium methoxide in methanol (0.5 M, 72.2 mL, 36.1 mmol, 2 equiv) was added to 4-chloro-2-(2,6-diethyl-phenyl)-7,8-dihydro-6H-quinolin-5-one (5.67 g, 18.1 mmol, 1 equiv) and the resulting mixture was heated in a 65° C. oil bath for 45 minutes. The mixture was then concentrated and the residue was purified by flash column chromatography (SiO₂, 5-35% EtOAc in hexanes) to yield 2-(2,6-diethyl-phenyl)-4-methoxy-7,8-dihydro-6H-quinolin-5-one (4.74 g, 85%).

¹H NMR (300 MHz, CDCl₃) δ 7.27-7.34 (m, 1H), 7.15 (d, 2H), 6.78 (s, 1H), 3.93 (s, 3H), 3.15 (t, 2H), 2.71 (t, 2H), 2.30-2.44 (m, 4H), 2.13-2.21 (m, 2H), 1.09 (t, 6H); MS m/z (MH⁺) 310.1.

Sodium borohydride (440 mg, 11.6 mmol, 1.1 equiv) was added to a solution of 2-(2,6-diethyl-phenyl)-4-methoxy-7,8-dihydro-6H-quinolin-5-one (3.27 g, 10.6 mmol, 1 equiv) in MeOH (50 mL). The resulting solution was stirred at room temperature for 1.5 h. Excess hydride was quenched by addition of water (50 mL) and the resulting mixture was partially concentrated to remove MeOH. The remaining aqueous phase was extracted with EtOAc (3×50 mL). The organic phase was dried over Na₂SO₄, then was filtered and concentrated. The residue was purified by flash column chromatography (SiO₂, 25-75% EtOAc in hexanes) to yield 2-(2,6-diethyl-phenyl)-4-methoxy-5,6,7,8-tetrahydro-quinolin-5-ol as a white solid (3.23 g, 98%).

¹H NMR (300 MHz, CDCl₃) δ 7.24-7.31 (m, 1H), 7.10-7.16 (m, 2H), 6.65 (s, 1H), 5.11-5.17 (m, 1H), 3.90 (s, 3H), 2.94-3.05 (m, 1H), 2.76-2.89 (m, 2H), 2.27-2.45 (m, 4H), 1.80-2.14 (m, 4H), 1.08 (t, 3H), 1.07 (t, 3H); MS m/z (MH⁺) 312.1.

Thionyl chloride (17.8 mL, 244 mmol, 50 equiv) was added to a solution of 2-(2,6-diethyl-phenyl)-4-methoxy-5,6,7,8-tetrahydro-quinolin-5-ol (1.52 g, 4.88 mmol, 1 equiv) in CH₂Cl₂ (35 mL). The resulting light yellow solution was stirred at ambient temperature for 1 h, then was concentrated. The residue was partitioned between CH₂Cl₂ (30 mL) and an ice-cold saturated aqueous solution of NaHCO₃ (30 mL). The separated aqueous phase was extracted with two 30-mL portions of CH₂Cl₂. The organic extracts were dried (Na₂SO₄), filtered, and concentrated to yield 5-chloro-2-(2,6-diethyl-phenyl)-4-methoxy-5,6,7,8-tetrahydro-quinoline, which was used without further purification in the next step. MS m/z (MH⁺) 330.2.

To the 5-chloro-2-(2,6-diethyl-phenyl)-4-methoxy-5,6,7,8-tetrahydro-quinoline was added CH₃CN (10 mL), K₂CO₃ (2.70 g, 19.5 mmol, 4 equiv), and N-ethyl-1-naphthylamine (3.15 mL, 19.5 mmol, 4 equiv). The resulting brown suspension was stirred at room temperature for 4 d. The reaction mixture was partitioned between EtOAc (50 mL) and water (50 mL). The separated aqueous phase was extracted with EtOAc (2×50 mL). The organic extracts were dried (Na₂SO₄), filtered, and concentrated. The residue was isolated by flash column chromatography (SiO₂, 0-20% EtOAc in hexanes) followed by reverse phase chromatography (10-90% CH₃CN in water, 0.05% TFA) to yield the title compound as its corresponding TFA salt. The corresponding free base of the title compound was obtained following partial concentration of the HPLC eluant to remove CH₃CN, basification of the remaining aqueous solution to pH 8 (by addition of a saturated aqueous solution of NaHCO₃), and extraction with CH₂Cl₂ (3×200 mL). The organic extracts were dried (Na₂SO₄), filtered, and concentrated to yield [2-(2,6-diethyl-phenyl)-4-methoxy-5,6,7,8-tetrahydro-quinolin-5-yl]-ethyl-naphthlen-1-yl-amine (0.646 g, 29%).

¹H NMR (300 MHz, CD₃OD) δ 8.47-8.54 (m,1H), 7.73-7.79 (m, 1H), 7.56-7.63 (m, 2H), 7.35-7.48 (m, 3H), 7.24 (t, 1H), 7.05-7.13 (m, 2H), 6.55 (s, 1H), 4.96 (t, 1H), 3.73 (s, 3H), 3.05-3.28 (m, 3H), 2.71-2.84 (m, 1H), 2.19-2.53 (m, 4H), 1.95-2.09 (m,1H), 1.71-1.93 (m, 2H), 1.50-1.63 (m,1H), 1.04 (t, 3H), 0.92 (t, 3H), 0.82 (t, 3H); MS m/z (MH⁺) 465.3.

### Example 2

### (±)-[2-(2,6-Diethyl-phenyl)-4-methoxy-5,6,7,8-tetrahydro-quinolin-5-yl]-methyl-naphthlen-1-yl-amine (Compound #20)

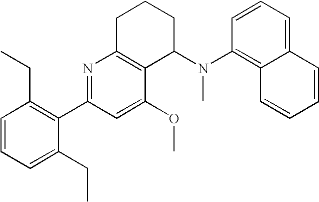

[2-(2,6-Diethyl-phenyl)-4-methoxy-5,6,7,8-tetrahydro-quinolin-5-yl]-naphthlen-1-yl-amine was prepared as described in Example 1 above, substituting 1-naphthylamine for N-ethyl-1-naphthylamine.

To a solution of the trifluoroacetate salt of [2-(2,6-diethyl-phenyl)-4-methoxy-5,6,7,8-tetrahydro-quinolin-5-yl]-naphthlen-1-yl-amine 10 mg, 18.2 μmol, 1 equiv) in CH₃CN (100 μL) was added in sequence 37% aqueous formaldehyde solution (15.4 μL, 206 μmol, 9 equiv) and sodium cyanoborohydride (4.3 mg, 68.7 μmol, 3 equiv). The mixture was cooled in an ice-bath and acetic acid (3.3 μL, 57.3 μmol, 2.5 equiv) was added. After 5 min, the mixture was allowed to warm to room temperature, and stirring was continued for an additional 1.5 h. The reaction mixture was concentrated and the residue was taken up in water (5 mL). The aqueous mixture was extracted with Et₂O (3×5 mL). The organic extracts were dried (Na₂SO₄), filtered, and concentrated. The residue was purified by flash column chromatography (SiO₂, 10-20% EtOAc in hexanes) to yield the title compound (4.6 mg, 56%).

¹H NMR (300 MHz, CD₃OD) δ 8.38-8.43 (m,1H), 7.77-7.82 (m,1H), 7.55-7.60 (m, 1H), 7.38-7.50 (m, 4H), 7.27 (t, 1H), 7.09-7.15 (m, 2H), 6.63 (s, 1H), 5.05 (t, 1H), 3.67 (s, 3H), 3.10 (ddd, 1H), 2.74-2.87 (m, 1H), 2.72 (s, 3H), 1.99-2.45 (m, 6H), 1.60-1.78 (m, 2H), 1.06 (t, 3H), 0.99 (t, 3H); MS m/z (MH⁺) 451.2.

### Example 3

### (±)-Ethyl-[2-(2-ethyl-phenyl)-4-methoxy-5,6,7,8-tetrahydro-quinolin-5-yl]-naphthlen-1-yl-amine (Compound #39)

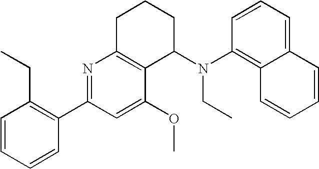

A solution of sodium methoxide in methanol (0.5 M, 20.7 mL, 10.4 mmol, 1.05 equiv) was added to 2,4-dichloro-7,8-dihydro-6H-quinolin-5-one (2.13 g, 9.86 mmol, 1 equiv). The resulting suspension was stirred at room temperature for 24 h. The reaction mixture was concentrated and the residue was purified by flash column chromatography (SiO₂, 0-40% EtOAc in hexanes) to yield 2-chloro-4-methoxy-7,8-dihydro-6H-quinolin-5-one as a white solid.

Analysis of a representative sample of the prepared product indicated contamination by a small amount (7% by ¹H NMR integration) of the bis-substitution product 2,4-dimethoxy-7,8-dihydro-6H-quinolin-5-one. The isolated material was used without further purification in the next reaction.

¹H NMR (300 MHz, CDCl₃) δ 6.81 (s,1H), 3.96 (s, 3H), 3.07 (t, 2H), 2.64 (t, 2H), 2.06-2.15 (m, 2H); MS m/z (MH⁺) 212.0.

To a solution of 2-chloro-4-methoxy-7,8-dihydro-6H-quinolin-5-one in MeOH (28 mL) was added sodium borohydride (238 mg, 6.28 mmol, 1.1 equiv) (gas evolution was observed). The resulting mixture was stirred at room temperature for 1 hour before addition of water (10 mL) to quench excess hydride reagent. The resulting mixture was partially concentrated, to remove organic solvent. The residual aqueous mixture was diluted with water (10 mL) and extracted with CH₂Cl₂ (3×20 mL). The organic extracts were dried (Na₂SO₄), filtered, and concentrated. The residue was purified by flash column chromatography (SiO₂, 30-70% EtOAc in hexanes), to yield 2-chloro-4-methoxy-5,6,7,8-tetrahydro-quinolin-5-ol (919 mg, 44% over two steps) as a white foamy solid.

¹H NMR (300 MHz, CDCl₃) δ 6.70 (s, 1H), 4.98-5.03 (m, 1 H), 3.93 (s, 3H), 2.92 (dt, 1H), 2.74 (ddd, 1H), 2.58 (d, 1H), 1.92-2.08 (m, 2H), 1.74-1.89 (m, 2H); MS m/z (MH⁺) 214.0.

Thionyl chloride (15.7 mL, 215 mmol, 50 equiv) was added to a solution of 2-chloro-4-methoxy-5,6,7,8-tetrahydro-quinolin-5-ol (0.919 g, 4.30 mmol, 1 equiv) in CH₂Cl₂ (30 mL). The resulting solution was stirred at ambient temperature for 4.5 h, then concentrated. The residue was partitioned between CH₂Cl₂ (20 mL) and an ice-cold saturated aqueous solution of NaHCO₃ (20 mL). The separated aqueous phase was extracted with two 20-mL portions of CH₂Cl₂. The organic extracts were dried (Na₂SO₄), filtered, and concentrated to yield 2,5-dichloro-4-methoxy-5,6,7,8-tetrahydro-quinoline, which was used without further purification in the next step.

¹H NMR (300 MHz, CDCl₃) δ 6.68 (s, 1H), 5.38-5.42 (m, 1H), 3.94 (s, 3H), 3.01 (ddt, 1H), 2.73-2.87 (m, 1H), 2.21-2.37 (m, 2H), 1.90-2.04 (m, 2H); MS m/z (MH⁺) 232.0.

To the 2,5-dichloro-4-methoxy-5,6,7,8-tetrahydro-quinoline was added CH₃CN (10 mL), K₂CO₃ (2.38 g, 17.2 mmol, 4 equiv), and N-ethyl-1-naphthylamine (2.78 mL, 17.2 mmol, 4 equiv). The resulting brown suspension was stirred at reflux for 8 hours. The reaction mixture was partitioned between EtOAc (20 mL) and water (20 mL). The separated aqueous phase was extracted with EtOAc (2×20 mL). The organic extracts were dried (Na₂SO₄), filtered, and concentrated. The resulting residue was isolated by flash column chromatography (SiO₂, 0-20% EtOAc in hexanes) to yield (2-chloro-4-methoxy-5,6,7,8-tetrahydro-quinolin-5-yl)-ethyl-naphthlen-1-yl-amine (0.244 g, 15%; first of isomeric compounds to elute) as a white foam.

¹H NMR (300 MHz, CDCl₃) δ8.48-8.55 (m, 1H), 7.77-7.84 (m, 1H), 7.61 (dd, 1H), 7.39-7.47 (m, 4H), 6.65 (s, 1H), 4.89 (t, 1H), 3.81 (s, 3H) 3.03-3.16 (m, 2H), 2.73-2.94 (m, 2H), 2.07-2.23 (m, 1H), 1.91-2.01 (m, 1H), 1.57-1.70 (m, 1H), 1.37-1.50 (m, 1H), 0.76 (t, 3H); MS m/z (MH⁺) 367.1.

To (2-chloro-4-methoxy-5,6,7,8-tetrahydro-quinolin-5-yl)-ethyl-naphthlen-1-yl-amine (20.9 mg, 57.0 μmol, 1 equiv) was added 2-ethylphenylboronic acid (12.8 mg, 85.5 μmol, 1.5 equiv), PdCl₂(PPh₃)₂ (2.0 mg, 2.8 μmol, 0.05 equiv), an aqueous solution of Na₂CO₃ (2 M, 200 μL), and CH₃CN (200 μL). The resulting mixture was heated by microwave irradiation (130° C., 15 minutes, 250 W). The mixture was then partially concentrated, to remove organic solvent. The residual mixture was diluted with water (2 mL) and extracted with CH₂Cl₂ (3×2 mL). The organic extracts were dried, filtered, and concentrated, The title compound (12.9 mg, 52%) was isolated from the residue by flash column chromatography (SiO₂, 0-20% EtOAc in hexanes).

¹H NMR (300 MHz, CDCl₃) δ 8.57-8.63 (m, 1H), 7.78-7.82 (m, 1H), 7.61 (dd, 1H), 7.40-7.51 (m, 4H), 7.21-7.36 (m, 4H), 6.65 (s, 1H), 4.99 (t, 1H), 3.81 (s, 3H), 3.12-3.25 (m, 2H), 2.82-3.07 (m, 2H), 2.64 (q, 2H), 2.17-2.33 (m, 1H), 1.99-2.10 (m, 1H), 1.65-1.77 (m, 1H), 1.47-1.60 (m, 1H), 1.12 (t, 3H), 0.81 (t, 3H); MS m/z (MH⁺) 437.2.

### Example 4

### (±)-[2-(2,6-Diethyl-Phenyl)-4-ethoxy-5,6,7,8-tetrahydro-quinolin-5-yl]-ethyl-naphthlen-1-yl-amine (Compound #8)

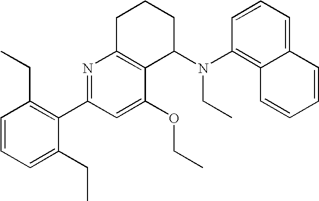

To a solution of 4-chloro-2-(2,6-diethyl-phenyl)-7,8-dihydro-6H-quinolin-5-one (351 mg, 1.12 mmol, 1.0 equiv) in MeOH (5 mL) was added NaBH₄ (46.5 mg, 1.23 mmol, 1.1 equiv). The resulting mixture was stirred at room temperature for 30 minutes. Excess hydride was quenched by addition of water (20 mL) and the mixture was partially concentrated, to remove organic solvent. The mixture was extracted with EtOAc (3×20 mL). The organic extracts were dried, filtered, and concentrated. The residue was purified by flash column chromatography (SiO₂, 0-30% EtOAc in hexanes) to yield 4-chloro-2-(2,6-diethyl-phenyl)-5,6,7,8-tetrahydro-quinolin-5-ol (274 mg), which was used as such in the next reaction. MS m/z (MH⁺) 316.1.

To a portion of 4-chloro-2-(2,6-diethyl-phenyl)-5,6,7,8-tetrahydro-quinolin-5-ol (162 mg) in CH₂Cl₂ (4 mL) was added thionyl chloride (1.87 mL, 25.6 mmol). The resulting yellow solution was stirred at room temperature for 30 min, then was concentrated. The residue was partitioned between CH₂Cl₂ (5 mL) and an ice-cold saturated aqueous solution of NaHCO₃ (5 mL). The separated aqueous phase was extracted with two 5-mL portions of CH₂Cl₂. The organic extracts were dried (Na₂SO₄), filtered, and concentrated, to yield crude 4,5-dichloro-2-(2,6-diethyl-phenyl)-5,6,7,8-tetrahydro-quinoline, which was used directly in the next reaction. MS m/z (MH⁺) 334.0.

To 5-dichloro-2-(2,6-diethyl-phenyl)-5,6,7,8-tetrahydro-quinoline was added CH₃CN (1.0 mL), K₂CO₃ (283 mg, 2.05 mmol), and N-ethyl-1-naphthylamine (0.330 mL, 2.05 mmol). The resulting suspension was stirred in a sealed tube heated at 110° C. (oil bath) for 3 d. The reaction mixture was diluted with water (20 mL) and was extracted with EtOAc (3×20 mL). The organic extracts were dried, filtered, and concentrated. Reverse phase chromatography (50-95% CH₃CN in water, 0.05% TFA) yielded the title compound as its corresponding TFA salt, which was then converted to the free base as follows. The eluant was partially concentrated, basified to pH 8 with saturated aqueous NaHCO₃, and extracted with CH₂Cl₂. The organic extracts were dried (Na₂SO₄), filtered, and concentrated, to yield [4-chloro-2-(2,6-diethyl-phenyl)-5,6,7,8-tetrahydro-quinolin-5-yl]-ethyl-naphthalen-1-yl-amine (43.2 mg).

¹H NMR (300 MHz, CDCl₃) δ 8.41-8.48 (m, 1H), 7.70-7.83 (m, 1H), 7.61-7.68 (m, 2H), 7.39-7.51 (m, 3H), 7.23-7.30 (m, 1H), 7.06-7.16 (m, 3H), 5.10 (t, 1H), 3.33 (ddd, 1H), 3.08-3.23 (m, 2H), 2.91 (dt, 1H), 2.05-2.49 (m, 6H), 1.49-1.77 (m, 2H), 1.07 (t, 3H), 0.99 (t, 3H), 0.83 (t, 3H); MS m/z (MH⁺) 469.2.

[4-Chloro-2-(2,6-diethyl-phenyl)-5,6,7,8-tetrahydro-quinolin-5-yl]-ethyl-naphthlen-1-yl-amine (10.8 mg, 23.0 μmol) was heated by microwave irradiation (155° C., 75 W, 2 h) in a solution of sodium ethoxide in EtOH (21 wt. %, 1.0 mL). The reaction mixture was concentrated and the residue was partitioned between CH₂Cl₂ (5 mL) and water (5 mL). The separated aqueous phase was extracted with CH₂Cl₂ (2×5 mL). The organic extracts were dried (Na₂SO₄), filtered, and concentrated. Reverse phase chromatography (10-90% CH₃CN in water, 0.05% TFA) yielded the title compound as its corresponding TFA salt, which was then converted to the free base as follows. The eluant was partially concentrated, basified to pH 8 with saturated aqueous NaHCO₃, and extracted with CH₂Cl₂. The organic extracts were washed with water, dried (Na₂SO₄), filtered, and concentrated to yield the title compound (6.6 mg, 60%).

¹H NMR (300 MHz, CDCl₃) δ 8.63-8.70 (m,1H), 7.77-7.83 (m,1H), 7.61 (d, 1H), 7.56 (d, 1H), 7.41-7.47 (m, 3H), 7.23-7.28 (m, 1H), 7.08-7.15 (m, 2H), 6.54 (s,1H), 5.05 (t, 1H), 3.95-4.11 (m, 2H), 2.99-3.29 (m, 3H), 2.79-2.93 (m, 1H), 1.98-2.48 (m, 6H), 1.68-1.80 (m, 1H), 1.43-1.55 (m, 4H), 1.09 (t, 3H), 1.02 (t, 3H), 0.82 (t, 3H); MS m/z (MH⁺) 479.2.

### Example 5

### (±)-[2-(2,6-Diethyl-phenyl )-4-ethyl-5,6,7,8-tetrahydro-quinolin-5-yl]-(5-methoxy-2-methyl-phenyl)-amine (Compound #25)

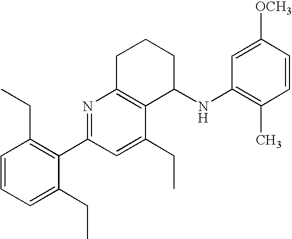

A mixture of 4-chloro-2-(2,6-diethyl-phenyl)-7,8-dihydro-6H-quinolin-5-one (109 mg, 346 μmol, 1 equiv), tributyl(vinyl)stannane (111 μL, 381 μmol, 1.1 equiv), and Pd(PPh₃)₄ (20 mg, 17.3 μmol. 0.05 equiv) in 1,4-dioxane (1.5 mL) was heated at reflux for 45 minutes. The reaction mixture was allowed to cool and was diluted with 28-30% ammonium hydroxide solution: water (1:1 v:v, 5 mL). The resulting mixture was extracted with CH₂Cl₂ (3×5 mL). The organic extracts were dried (Na₂SO₄), filtered, and concentrated. The resulting residue was used directly in the next reaction. MS m/z (MH⁺) 306.2

To a solution of the residue prepared as above in EtOH (1.5 mL) was added NaBH₄ (17.0 mg, 450 μmol). The resulting solution was stirred at room temperature for 10 minutes. The mixture was concentrated and the residue was partitioned between CH₂Cl₂ (5 mL) and water (5 mL). The separated aqueous phase was extracted with CH₂Cl₂ (2×5 mL). The organic extracts were dried (Na₂SO₄), filtered, and concentrated. 2-(2,6-Diethyl-phenyl)-4-ethyl-5,6,7,8-tetrahydro-quinolin-5-ol (65 mg, 61%) was isolated by flash column chromatography (10-50% EtOAc in heptane). MS m/z (MH⁺) 310.2.

Thionyl chloride (154 μL, 2.11 mmol, 10 equiv) was added to a solution of 2-(2,6-diethyl-phenyl)-4-ethyl-5,6,7,8-tetrahydro-quinolin-5-ol (65.1 mg, 211 μmol, 1 equiv) in CH₂Cl₂ (1.5 mL). The resulting colorless solution was stirred at ambient temperature for 30 min, then concentrated. The residue was partitioned between CH₂Cl₂ (5 mL) and an ice-cold saturated aqueous solution of NaHCO₃ (5 mL). The separated aqueous phase was extracted with two 5-mL portions of CH₂Cl₂. The organic extracts were dried (Na₂SO₄), filtered, and concentrated to yield 5-chloro-2-(2,6-diethyl-phenyl)-4-ethyl-5,6,7,8-tetrahydro-quinoline. The isolated product was used without further purification in the next step. MS m/z (MH⁺) 328.2.

The 5-chloro-2-(2,6-diethyl-phenyl)-4-ethyl-5,6,7,8-tetrahydro-quinoline prepared as in the previous step was dissolved in CH₃CN (1.0 mL). Half of this solution was transferred to a pressure tube and K₂CO₃ (58.3 mg, 422 μmol, 4 equiv) and 5-methoxy-2-methyl-phenylamine (57.9 mg, 422 μmol, 4 equiv) were added. The sealed tube was heated in a 50° C. oil bath for 3 d. The reaction mixture was diluted with water (5 mL) and was extracted with EtOAc (3×5 mL). The organic extracts were dried (Na₂SO₄), filtered, and concentrated. The residue was subjected to reverse phase chromatography (10-90% CH₃CN in water, 0.05% TFA) to yield the title compound as its corresponding TFA salt (21.0 mg, 46%). The title compound, as its corresponding free base was obtained from the HPLC eluant as described Example 4, above.

¹H NMR (400 MHz, CD₃OD) δ 7.29 (t, 1H), 7.13-7.18 (m, 2H), 7.07 (s, 1H), 6.91 (d, 1H), 6.43 (d, 1H), 6.19 (dd, 1H), 4.90-4.93 (m, 1H), 3.76 (s, 3H), 3.03 (dd, 1H), 2.63-2.88 (m, 3H), 2.21-2.43 (m, 5H), 1.99-2.12 (m, 1H), 2.01 (s, 3H), 1.83-1.91 (m, 1H), 1.67-1.77 (tt, 1H), 1.21 (t, 3H), 1.07 (t, 3H), 1.02 (t, 3H). MS m/z (MH⁺) 429.3.

### Example 6

### (±)-[2-(2,6-Diethyl-phenyl)-4-methoxy-5-propyl-5,6,7,8-tetrahydro-quinolin-5-yl]-(5-methoxy-2-methyl-phenyl)-amine (Compound #70)

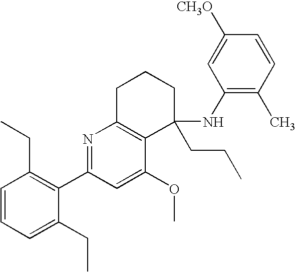

To a solution of 2-(2,6-diethyl-phenyl)-4-methoxy-7,8-dihydro-6H-quinolin-5-one (50.7 mg, 164 mmol, 1 equiv) in THF (1.0 mL) at −78° C. was added a solution of n-propyl magnesium chloride in Et₂O (2.0 M, 246 μL, 492 μmol, 3.0 equiv). The reaction mixture was removed from the cooling bath and was stirred for 20 minutes. Excess Grignard reagent was quenched by addition of saturated aqueous ammonium chloride solution (5 mL). The mixture was extracted with EtOAc (3×5 mL). The organic extracts were dried (Na₂SO₄), filtered, and concentrated to yield a residue which was used directly in the next reaction. MS m/z (MH⁺) 354.2.

One-half of the residue prepared as described above was dissolved in CH₂Cl₂ (0.5 mL) and thionyl chloride (60 μL, 0.82 mmol, 10 equiv) was added. The resulting solution was stirred for 30 min at room temperature. The reaction mixture was concentrated. To the residue was then added CH₃CN (300 μL), K₂CO₃ (45.3 mg, 328 μmol, 4 equiv), and 5-methoxy-2-methyl-phenylamine (44.9 mg, 328 μmol, 4 equiv). The resulting suspension was stirred at room temperature for 4 d. The reaction mixture was diluted with water (5 mL) and was extracted with EtOAc (3×5 mL). The organic extracts were dried (Na₂SO₄), filtered, and concentrated. The residue was purified by reverse phase chromatography (10-90% CH₃CN in water, 0.05% TFA) to yield the title compound as its corresponding TFA salt (3.1 mg, 8%). The free base of the title compound was obtained from the HPLC eluant according to the procedure described in Example 4, above.

¹H NMR (300 MHz, CD₃OD) δ 7.26 (t,1H), 7.09-7.16 (m, 2H), 6.85 (d, 1H), 6.60 (s, 1H), 6.05 (dd, 1H), 5.79 (d, 1H), 3.59 (s, 3H), 3.51 (s, 3), 2.98-3.04 (m, 1H), 1.96-2.54 (m, 14H), 1.72-1.84 (m, 2H), 1.08 (t, 3H), 1.03 (t, 3H), 0.96 (t, 3H); MS m/z (MH⁺) 473.3.

### Example 7

### (±)-[2-(2,6-Diethyl-phenyl)-5,6,7,8-tetrahydro-quinolin-5-yl]-ethyl-naphthlen-1-yl-amine (Compound #109)

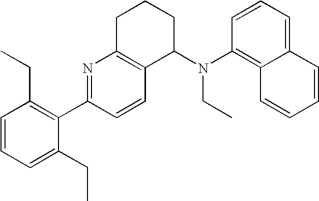

7,8-Dihydro-1H,6H-quinoline-2,5-dione (101 mg, 0.621 mmol, 1 equiv) was treated with POCl₃ (1.0 mL, 10.9 mmol, 18 equiv) and the resulting suspension was heated in a 90° C. oil bath for 40 minutes. The resulting mixture was concentrated and the residue was poured into ice (5 mL). The resulting suspension was neutralized to pH 7 by addition of solid Na₂CO₃ and was extracted with CH₂Cl₂ (3×5 mL). The organic phase was washed with water (15 mL), then was dried (Na₂SO₄), filtered, and concentrated. The residue was purified by flash column chromatography (SiO₂, 0-20% EtOAc in hexanes) to yield 2-chloro-7,8-dihydro-6H-quinolin-5-one as a white solid (74.3 mg, 66%).

¹H NMR (300 MHz, CDCl₃) δ 8.23 (d, 1H), 7.31 (d, 1H), 3.14 (t, 2H), 2.69 (t, 2H), 2.20 (quint, 2H); MS m/z (MH⁺) 182.1.

To a mixture of 2-chloro-7,8-dihydro-6H-quinolin-5-one (68.8 mg, 0.379 mmol, 1 equiv), 2,6-diethylphenylboronic acid (70.8 mg, 0.398 mmol, 1.05 equiv) and Pd(PPh₃)₄ (21.9 mg, 18.9 μmol, 0.05 equiv) was added 2 M aqueous Na₂CO₃ (1 mL) and toluene (1 mL). The resulting mixture was heated at reflux under nitrogen for 1 d. Analysis of a sample of the reaction mixture by LCMS indicated incomplete conversion, so additional portions of 2,6-diethylphenylboronic acid, Pd(PPh₃)₄, 2 M aqueous Na₂CO₃, and toluene (amounts as above) were added and the resulting mixture was heated at reflux for an additional 4 h. The mixture was then partitioned between EtOAc (20 mL) and water (20 mL). The separated aqueous phase was extracted with two additional 20-mL portions of EtOAc. The organic extracts were dried (Na₂SO₄) and filtered, and the filtrate was concentrated. The resulting residue was purified by flash column chromatography (SiO₂, 0-20% EtOAc in hexanes) to yield 2-(2,6-diethyl-phenyl)-7,8-dihydro-6H-quinolin-5-one (75.1 mg, 71%).

¹H NMR (300 MHz, CDCl₃) δ 8.34 (d, 1H), 7.23-7.34 (m, 2H), 7.15 (d, 2H), 3.21 (t, 2H), 2.75 (t, 2H), 2.33 (q, 4H), 2.26 (quint, 2H), 1.05 (t, 6H); MS m/z (MH⁺) 280.3.

To a solution of 2-(2,6-diethyl-phenyl)-7,8-dihydro-6H-quinolin-5-one (75.1 mg, 0.269 mmol, 1 equiv) in MeOH (1 mL) was added NaBH₄ (11.2 mg, 0.296 mmol, 1.1 equiv). The resulting solution was stirred at room temperature for 20 minutes. Excess hydride was quenched by addition of water (5 mL) and the resulting mixture was partially concentrated, to remove organic solvent. The residual aqueous mixture was extracted with EtOAc (3×5 mL). The organic extracts were dried (Na₂SO₄), filtered, and concentrated. The residue was purified by flash column chromatography (SiO₂, 25-75% EtOAc in hexanes) to yield 2-(2,6-diethyl-phenyl)-5,6,7,8-tetrahydro-quinolin-5-ol as a colorless oil (64.0 mg, 85%).

¹H NMR (300 MHz, CDCl₃) δ 7.73 (d, 1H), 7.27 (t, 1H), 7.12 (d, 2H), 7.08 (d, 1H), 4.63-4.69 (m, 1H), 3.28 (br s, 1H), 2.85-3.06 (m, 2H), 2.22-2.42 (m, 4H), 1.95-2.13 (m, 2H), 1.73-1.90 (m, 2H), 1.04 (t, 6H); MS m/z (MH⁺) 282.3.

Thionyl chloride (830 μL, 11.4 mmol, 50 equiv) was added to a solution of 2-(2,6-diethyl-phenyl)-5,6,7,8-tetrahydro-quinolin-5-ol (64.0 mg, 227 □mol, 1 equiv) in CH₂Cl₂ (2 mL). The resulting colorless solution was stirred at ambient temperature for 1.5 hours, then concentrated. The resulting residue was partitioned between CH₂Cl₂ (5 mL) and an ice-cold saturated aqueous solution of NaHCO₃ (5 mL). The separated aqueous phase was extracted with two 5-mL portions of CH₂Cl₂. The organic extracts were dried (Na₂SO₄), filtered, and concentrated to yield 5-chloro-2-(2,6-diethyl-phenyl)-5,6,7,8-tetrahydro-quinoline as a residue, which was used without further purification in the next step. MS m/z (MH⁺) 300.1.

To the 5-chloro-2-(2,6-diethyl-phenyl)-5,6,7,8-tetrahydro-quinoline was added CH₃CN (1 mL), K₂CO₃ (126 mg, 0.910 mmol, 4 equiv), and N-ethyl-1-naphthylamine (147 μL, 0.910 mmol, 4 equiv). The resulting brown suspension was stirred at room temperature for 20 hours, then was heated to reflux and was stirred for an additional 2 d. The reaction mixture was partitioned between EtOAc (5 mL) and water (5 mL). The separated aqueous phase was extracted with EtOAc (2×5 mL). The organic extracts were dried (Na₂SO₄), filtered, and concentrated. The resulting residue was purified by reverse phase chromatography (10-90% CH₃CN in water, 0.05% TFA) to yield the title compound as its corresponding TFA salt. The title compound, as its corresponding free base product was obtained following partial concentration of the HPLC eluant to remove CH₃CN, basification of the remaining aqueous solution to pH 8 (by addition of a saturated aqueous solution of NaHCO₃), and extraction with CH₂Cl₂ (3×100 mL). The organic extracts were then dried (Na₂SO₄), filtered, and concentrated to yield the title compound (15.9 mg, 23%) as a colorless oil.

¹H NMR (400 MHz, CDCl₃) δ 8.57 (d, 1H), 8.34-8.39 (m, 1H), 7.82-7.87 (m, 1H), 7.61 (d, 1H), 7.43-7.49 (m, 3H), 7.40 (d, 1H), 7.26-7.33 (m, 2H), 7.16 (d, 2H), 4.57 (dd, 1H), 3.41-3.50 (m, 1H), 2.82-2.99 (m, 3H), 2.25-2.44 (m, 4H), 1.99-2.09 (m, 1H), 1.86-1.99 (m, 2H), 1.52-1.64 (m, 1H), 1.07 (t, 3H), 1.05 (t, 3H), 1.01 (t, 3H); MS m/z (MH⁺) 435.3.

### Example 8

### (±)-[2-(2,6-Diethyl-phenyl)-4-methoxy-1-oxy-5,6,7,8-tetrahydro-quinolin-5-yl]-ethyl-naphthlen-1-yl-amine (Compound #19)

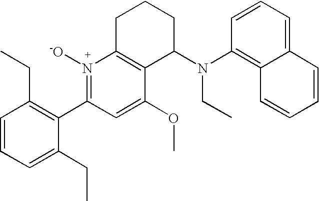

To a solution of (±)-ethyl-[2-(2-ethyl-phenyl)-4-methoxy-5,6,7,8-tetrahydro-quinolin-5-yl]-naphthlen-1-yl-amine (12.9 mg, 27.8 μmol, 1 equiv) in CH₂Cl₂ (300 μL) at 0° C. was added mCPBA (7.5 mg, ≦77%, 33.3 μmol, 1.2 equiv). The resulting mixture was allowed to slowly warm to room temperature and was stirred for 3 d, then was partitioned between CH₂Cl₂ (5 mL) and a saturated aqueous solution of NaHCO₃ (5 mL). The separated aqueous phase was extracted with CH₂Cl₂ (5 mL). The organic extracts were washed with saturated aqueous solution of NaHCO₃ (5 mL) and water (5 mL), and were dried (Na₂SO₄), filtered, and concentrated. The resulting residue was purified by flash column chromatography (SiO₂, 50-100% EtOAc in hexanes) to yield the title compound (5.9 mg, 44%).

¹H NMR (400 MHz, CD₃OD) δ8.33-8.38 (m,1H), 7.72-7.77 (m, 1H), 7.67 (d, 1H), 7.63 (d, 1H), 7.46 (t, 1H), 7.35-7.41 (m, 2H), 7.28 (t, 1H), 7.12 (d, 1H), 7.09 (d, 1H), 6.58 (s, 1H), 4.88-4.92 (m, 1H), 3.62 (s, 3H), 316-3.36 (m, 3H), 2.86-2.97 (m, 1H), 2.52-2.65 (m, 1H), 2.29-2.45 (m, 2H), 2.04-2.15 (m, 1H), 1.90-2.01 (m, 2H), 1.38-1.60 (m, 2H), 1.04 (t, 3H), 0.89 (t, 3H), 0.83 (t, 3H); MS m/z (MH⁺) 481.4.

Table 2, below, lists representative compounds of the present invention prepared according to the processes as described in Schemes 1-9 above, and the procedures as described in Examples 1-8 above. Also listed in Table 2 are the measured mass spectrum values measured for the prepared compounds.

TABLE 2
Compounds of Formula (I)

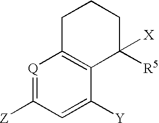

| ID | | | | | | MS |
|---|---|---|---|---|---|---|
| No. | Q | X | R⁵ | Y | Z | (MH⁺) |
| 1 | N | N-(2-hydroxy-ethyl- | H | isopropoxy | 2,6-diethyl- | 459.3 |
| | | phenyl)-amino- | | | phenyl | |
| 2 | N | N-(2-hydroxymethyl- | H | methoxy | 2,6-diethyl- | 485.2 |
| | | 5-trifluoromethyl- | | | phenyl | |
| | | phenyl)-amino- | | | | |
| 3 | N | N-(2-hydroxy-methyl- | H | methoxy | 2,6-diethyl- | 451.1 |
| | | 5-chloro-phenyl)- | | | phenyl | |
| | | amino- | | | | |
| 4 | N | (R)-N-ethyl- | H | methoxy | 2,6-diethyl- | ^{a} |
| | | (napthy-1-yl)-amino- | | | phenyl | |
| 5 | N | N-(2-hydroxyethyl- | H | methoxy | 2,6-diethyl- | 431.1 |
| | | phenyl)-amino- | | | phenyl | |
| 6 | N | N-ethyl-N-(naphth-1- | H | cyclopentyl- | 2,6-diethyl- | 519.3 |
| | | yl)-amino- | | oxy | phenyl | |
| 7 | N | N-(2-hydroxymethyl- | H | methoxy | 2,6-diethyl- | 435.1 |
| | | 4-fluoro-phenyl)- | | | phenyl | |
| | | amino- | | | | |
| 8 | N | N-ethyl-N-(naphth-1- | H | ethoxy | 2,6-diethyl- | 479.2 |
| | | yl)-amino- | | | phenyl | |
| 9 | N | N-(2-hydroxymethyl- | H | methoxy | 2,6-diethyl- | 451.2 |
| | | 4-chloro-phenyl)- | | | phenyl | |
| | | amino- | | | | |
| 10 | N | N-ethyl-N-(naphth-1- | H | methylthio | 2,6-diethyl- | 481.2 |
| | | yl)-amino- | | | phenyl | |
| 11 | N | N-(2-methyl-5- | H | methoxy | 2,6-diethyl- | 431.1 |
| | | methoxy-phenyl)- | | | phenyl | |
| | | amino- | | | | |
| 12 | N | N-ethyl-N-(napth-1- | H | methoxy | 2,6-diethyl- | 465.3 |
| | | yl)-amino- | | | phenyl | |
| 13 | N | N-ethyl-N-(napth-1- | H | methoxy | 2-chloro-6- | 473.2 |
| | | yl)-amino- | | | methoxy-phenyl | |
| 14 | N | N-(2-hydroxyethyl)-N- | H | methoxy | 2,6-diethyl- | 453.2 |
| | | (naphth-1-yl)-amino- | | | phenyl | |
| 15 | N | N-(2-hydroxymethyl- | H | methoxy | 2,6-diethyl- | 417.3 |
| | | phenyl)-amino- | | | phenyl | |
| 16 | N | N-(ethyl)-N-(naphth- | H | methoxy | 2,6-dimethyl | 437.2 |
| | | 1-yl)-amino- | | | phenyl | |
| 17 | N | N-(2-methyl-5-methoxy- | H | cyclopentyl- | 2,6-diethyl- | 485.2 |
| | | phenyl)-amino- | | oxy | phenyl | |
| 18 | N | N-(2-methyl-5- | H | isopropoxy | 2,6-diethyl- | 459.3 |
| | | methoxy-phenyl)- | | | phenyl | |
| | | amino- | | | | |
| 19 | N⁺O⁻ | N-ethyl-N-(naphth-1-yl)- | H | methoxy | 2,6-diethyl- | 481.4 |
| | | amino- | | | phenyl | |
| 20 | N | N-methyl-N-(naphth- | H | methoxy | 2,6-diethyl- | 451.2 |
| | | 1-yl)-amino- | | | phenyl | |
| 21 | N | N-ethyl-N-(naphth-1- | H | methoxy | 2,6-dimethyl-4- | 467.2 |
| | | yl)-amino- | | | methoxy-phenyl | |
| 22 | N | N-methyl-N-(6- | H | methoxy | 2,6-diethyl- | 481.3 |
| | | methoxy-naphth-1- | | | phenyl | |
| | | yl)-amino- | | | | |
| 23 | N | N-ethyl-N-(naphth-1- | H | ethyl | 2,6-diethyl- | 463.3 |
| | | yl)-amino- | | | phenyl | |
| 24 | N | N-ethyl-N-(5,6,7,8- | H | methoxy | 2,6-diethyl- | 469.3 |
| | | tetrahydronaphth-1- | | | phenyl | |
| | | yl)-amino- | | | | |
| 25 | N | N-(2-methyl-5-methoxy- | H | ethyl | 2,6-diethyl- | 429.3 |
| | | phenyl)-amino- | | | phenyl | |
| 26 | N | 2-(8-trifluoro-methyl- | H | methoxy | 2,6-diethyl- | 495.3 |
| | | 1,2,3,4- | | | phenyl | |
| | | tetrahydro- | | | | |
| | | isoquinolinyl) | | | | |
| 27 | N | N-ethyl-N-(naphth-1-yl)- | H | benzyloxy | 2,6-diethyl- | 541.2 |
| | | amino- | | | phenyl | |
| 28 | N | N-(2-methyl-5- | H | methoxy | 2,6-diethyl- | 469.2 |
| | | trifluoromethyl-phenyl)- | | | phenyl | |
| | | amino- | | | | |
| 29 | N | 2-methyl-5-methoxy- | H | methoxy | 2,6-diethyl- | 432.2 |
| | | phenyl-oxy- | | | phenyl | |
| 30 | N | N-ethyl-N-(naphth-1-yl)- | H | methoxy | 2-methyl-phenyl | 423.2 |
| | | amino- | | | | |
| 31 | N | 2-(1,2,3,4-tetrahydro- | H | methoxy | 2,6-diethyl- | 427.2 |
| | | isoquinolinyl) | | | phenyl | |
| 32 | N | N-(2-chloro-5-methoxy- | H | methoxy | 2,6-diethyl- | 451.2 |
| | | phenyl)-amino- | | | phenyl | |
| 33 | N | N-(2-carboxyphenyl)- | H | methoxy | 2,6-diethyl- | 431.2 |
| | | amino- | | | phenyl | |
| 34 | N | N-(2,5-dimethoxy- | H | methoxy | 2,6-diethyl- | 447.2 |
| | | phenyl)-amino- | | | phenyl | |
| 35 | N | N-(2,5-dimethyl- | H | methoxy | 2,6-diethyl- | 451.2 |
| | | phenyl)-amino- | | | phenyl | |
| 36 | N | N-methyl-N-(4-methoxy- | H | methoxy | 2,6-diethyl- | 481.3 |
| | | naphth-1-yl)-amino- | | | phenyl | |
| 37 | N | N-(2-methyl-5-methoxy- | H | methoxy | 2,6-dimethyl- | 403.1 |
| | | phenyl)-amino- | | | phenyl | |
| 38 | N | N-methyl-N-(2-methyl- | H | methoxy | 2,6-diethyl- | 445.3 |
| | | 5-methoxy-phenyl)- | | | phenyl | |
| | | amino- | | | | |
| 39 | N | N-ethyl-N-(naphth-1-yl)- | H | methoxy | 2-ethyl-phenyl | 437.2 |
| | | amino- | | | | |
| 40 | N | N-methyl-N-(2,5- | H | methoxy | 2,6-diethyl- | 429.3 |
| | | dimethyl-phenyl)-amino- | | | phenyl | |
| 41 | N | N-(2-ethyl-phenyl)- | H | methoxy | 2,6-diethyl- | 415.2 |
| | | amino- | | | phenyl | |
| 42 | N | N-methyl-N-(2- | H | methoxy | 2,6-diethyl- | 415.2 |
| | | methyl-phenyl)-amino- | | | phenyl | |
| 43 | N | (R)-N-methyl-N-(S)- | H | methoxy | 2,6-diethyl- | 455.3 |
| | | 1,2,3,4-tetrahydro- | | | phenyl | |
| | | naphth-1-yl)-amino | | | | |
| 44 | N | 2-(8-methoxy- | H | methoxy | 2,6-diethyl- | 485.3 |
| | | carbonyl-1,2,3,4- | | | phenyl | |
| | | tetrahydro- | | | | |
| | | isoquinolinyl)- | | | | |
| 45 | N | 2-(7-fluoro-1,2,3,4- | H | methoxy | 2,6-diethyl- | 445.3 |
| | | tetrahydro- | | | phenyl | |
| | | isoquinolinyl)- | | | | |
| 46 | N | N-(2-methyl-phenyl)- | H | methoxy | 2,6-diethyl- | 401.3 |
| | | amino- | | | phenyl | |
| 47 | N | N-methyl-N-(2- | H | methoxy | 2,6-diethyl- | 465.2 |
| | | chloro-5-methoxy- | | | phenyl | |
| | | phenyl)-amino- | | | | |
| 48 | N | N-methyl-N-(4-chloro- | H | methoxy | 2,6-dimethyl- | 457.2 |
| | | naphth-1-yl)-amino- | | | phenyl | |
| 49 | N | N-(cyanomethyl)-N- | H | methoxy | 2,6-dimethyl- | 448.2 |
| | | (naphth-1-y)-amino- | | | phenyl | |
| 50 | N | N-(2-methyl-5-fluoro- | H | methoxy | 2,6-diethyl- | 419.2 |
| | | phenyl)-amino- | | | phenyl | |
| 51 | N | 2-(7-chloro-1,2,3,4- | H | methoxy | 2,6-diethyl- | 461.2 |
| | | tetrahydro-isoquinolinyl) | | | phenyl | |
| 52 | N | N-methyl-N-(2-methyl- | H | methoxy | 2,6-diethyl- | 465.3 |
| | | naphth-1-yl)-amino- | | | phenyl | |
| 53 | N | N-(2-methyl-5-chloro- | H | methoxy | 2,6-diethyl- | 435.2 |
| | | phenyl)-amino- | | | phenyl | |
| 54 | N | N-methyl-N-(1,2,3,4- | H | methoxy | 2,6-diethyl- | 456.2 |
| | | tetrahydro-quinolin-1-yl) | | | phenyl | |
| 55 | N | N-(hydroxyethyl)-N- | H | methoxy | 2,6-dimethyl | 481.3 |
| | | (naphth-1-yl)-amino- | | | phenyl | |
| 56 | N | 2-(5-chloro-1,2,3,4- | H | methoxy | 2,6-diethyl- | 461.2 |
| | | tetrahydro- | | | phenyl | |
| | | isoquinolinyl) | | | | |
| 57 | N | N-ethyl-N-(naphth-1-yl)- | H | methoxy | 2-isopropyl- | 451.3 |
| | | amino- | | | phenyl | |
| 58 | N | N-ethyl-N-(naphth-1-yl)- | H | methoxy | 2,6- | 469.2 |
| | | amino- | | | dimethoxy- | |
| | | | | | phenyl | |
| 59 | N | (S)-N-methyl-N-(R)- | H | methoxy | 2,6-diethyl- | 455.3 |
| | | (1,2,3,4-tetrahydro- | | | phenyl | |
| | | naphth-1-yl)-amino- | | | | |
| 60 | N | N-ethyl-N-(naphth-1-yl)- | H | chloro | 2,6-diethyl- | 469.2 |
| | | amino- | | | phenyl | |
| 61 | N | N-methyl-N-(naphth-1- | H | methoxy | 2,6-dimethyl | 423.2 |
| | | yl)-amino- | | | phenyl | |
| 62 | N | N-(2-methyl-5-methoxy- | H | dimethyl- | 2,6-diethyl- | 488.3 |
| | | phenyl)-amino- | | amino- | phenyl | |
| | | | | ethoxy | | |
| 63 | N | N-(2-methyl-4-methoxy- | H | methoxy | 2,6-diethyl- | 431.3 |
| | | phenyl)-amino- | | | phenyl | |
| 64 | N | 2-hydroxyethyl-phenyl- | H | methoxy | 2,6-diethyl- | 432.2 |
| | | oxy- | | | phenyl | |
| 65 | N | 1-(1,2,3,4-tetrahydro- | H | methoxy | 2,6-diethyl- | 427.2 |
| | | quinolinyl) | | | phenyl | |
| 66 | N | N-(2-methyl-5-phenyl- | H | methoxy | 2,6-diethyl- | 477.3 |
| | | phenyl)-amino- | | | phenyl | |
| 67 | N | N-methyl-N-(2-methyl- | H | methoxy | 2,6-diethyl- | 449.3 |
| | | 5-chloro-phenyl)-amino- | | | phenyl | |
| 68 | N | N-methyl-(2,5- | H | methoxy | 2,6-diethyl- | 461.2 |
| | | dimethoxy-phenyl)- | | | phenyl | |
| | | amino- | | | | |
| 69 | N | N-methyl-N-(2-methyl | H | methoxy | 2,6-dimethyl- | 417.2 |
| | | 5-methoxy-phenyl)- | | | phenyl | |
| | | amino- | | | | |
| 70 | N | N-(2-methyl-5-methoxy- | n-propyl | methoxy | 2,6-diethyl- | 473.3 |
| | | phenyl)-amino- | | | phenyl | |
| 71 | N | N-(4-hydroxy-methyl- | H | methoxy | 2,6-diethyl- | — |
| | | pyrid-3-yl)-amino | | | phenyl | |
| 72 | N | N-methyl-N-(indan-4-yl) | H | methoxy | 2,6-diethyl- | 441.3 |
| | | | | | phenyl | |
| 73 | N | N-ethyl-N-(naphth-1-yl)- | H | methoxy | 2-fluoro- | 427.2 |
| | | amino- | | | phenyl | |
| 74 | N | N-(2-cyano-phenyl- | H | methoxy | 2,6-diethyl- | 412.2 |
| | | amino- | | | phenyl | |
| 75 | N | N-(2-aminomethyl- | H | methoxy | 2,6-diethyl- | 416.2 |
| | | phenyl)-amino- | | | phenyl | |
| 76 | N | N-ethyl-N-(2,3-dimethyl- | H | methoxy | 2,6-dimethyl- | 415.3 |
| | | phenyl)-amino- | | | phenyl | |
| 77 | N | (S)-N-methyl-N-(S)- | H | methoxy | 2,6-diethyl- | 455.2 |
| | | (1,2,3,4-tetrahydro- | | | phenyl | |
| | | naphth-1-yl)-amino- | | | | |
| 78 | N | N-ethyl-N-(naphth-1-yl)- | H | methoxy | 1-naphthyl | 459.2 |
| | | amino- | | | | |
| 79 | N | N-(methylcarbonyl)-N- | H | methoxy | 2,6-diethyl- | 479.3 |
| | | (naphth-1-yl)-amino- | | | phenyl | |
| 80 | N | N-methyl-N-(2-methyl- | H | methoxy | 2,6-diethyl- | 445.2 |
| | | 4-methoxy-phenyl)- | | | phenyl | |
| | | amino- | | | | |
| 81 | N | N-(3-methoxy-phenyl)- | H | methoxy | 2,6-diethyl- | 417.3 |
| | | amino- | | | phenyl | |
| 82 | N | N-methyl-N-phenyl- | H | methoxy | 2,6-diethyl- | 401.2 |
| | | amino- | | | phenyl | |
| 83 | N | (R)-N-methyl-N-(R)- | H | methoxy | 2,6-diethyl- | 455.2 |
| | | (1,2,3,4-tetrahydro- | | | phenyl | |
| | | naphth-1-yl)-amino- | | | | |
| 85 | N | N-ethyl-N-benzyl- | H | methoxy | 2,6-diethyl- | 429.2 |
| | | amino- | | | phenyl | |
| 86 | N | benzyloxy- | H | methoxy | 2,6-diethyl- | 402.2 |
| | | | | | phenyl | |
| 87 | N | N-methyl-N- | H | methoxy | 2,6-diethyl- | 429.3 |
| | | (phenylethyl)-amino- | | | phenyl | |
| 88 | N | N-(indan-2-yl)-amino- | H | methoxy | 2,6-diethyl- | 427.3 |
| | | | | | phenyl | |
| 89 | N | 2-(2,3-dihydro-1H- | H | methoxy | 2,6-diethyl- | 413.3 |
| | | isoindolyl) | | | phenyl | |
| 90 | N | N-methyl-N-(naphth- | H | methoxy | 2,6-diethyl- | 465.2 |
| | | 1-yl-methyl)-amino- | | | phenyl | |
| 91 | N | N-methyl-N-(phenyl- | H | methoxy | 2,6-diethyl- | 443.3 |
| | | n-propyl)-amino | | | phenyl | |
| 92 | N | N-methyl-N-(2-furyl- | H | methoxy | 2,6-diethyl- | 405.3 |
| | | methyl)-amino- | | | phenyl | |
| 93 | N | 1-(2,3-dihydro-1H- | H | methoxy | 2,6-diethyl- | 413.2 |
| | | indolyl) | | | phenyl | |
| 94 | N | N-methyl-N-(2- | H | methoxy | 2,6-diethyl- | 421.2 |
| | | thienyl-methyl)- | | | phenyl | |
| | | amino- | | | | |
| 95 | N | N-methyl-N-(indan-2- | H | methoxy | 2,6-diethyl- | 441.1 |
| | | yl)-amino- | | | phenyl | |
| 96 | N | 2-(7-cyano-1,2,3,4- | H | methoxy | 2,6-diethyl- | 452.3 |
| | | tetrahydro- | | | phenyl | |
| | | isoquinolinyl) | | | | |
| 97 | N | 2-(7-methoxy- | H | methoxy | 2,6-diethyl- | 485.3 |
| | | carbonyl-1,2,3,4- | | | phenyl | |
| | | tetrahydro- | | | | |
| | | isoquinolinyl) | | | | |
| 98 | N | 2-(6-methoxy- | H | methoxy | 2,6-diethyl- | 485.2 |
| | | carbonyl-1,2,3,4- | | | phenyl | |
| | | tetrahydro- | | | | |
| | | isoquinolinyl) | | | | |
| 99 | N | 2-(5-methoxy- | H | methoxy | 2,6-diethyl- | 485.3 |
| | | carbonyl-1,2,3,4- | | | phenyl | |
| | | tetrahydro- | | | | |
| | | isoquinolinyl) | | | | |
| 100 | N | 2-(7-methoxy- | H | methoxy | 2,6-diethyl- | 457.3 |
| | | 1,2,3,4-tetrahydro- | | | phenyl | |
| | | isoquinolinyl) | | | | |
| 101 | N | 2-(7-trifluoromethyl- | H | methoxy | 2,6-diethyl- | 495.3 |
| | | 1,2,3,4-tetrahydro- | | | phenyl | |
| | | isoquinolinyl) | | | | |
| 102 | N | 2-(6-trifluoromethyl- | H | methoxy | 2,6-diethyl- | 495.2 |
| | | 1,2,3,4-tetrahydro- | | | phenyl | |
| | | isoquinolinyl) | | | | |
| 103 | N | N-(naphth-1-yl)- | H | methoxy | 2,6-diethyl- | 437.2 |
| | | amino | | | phenyl | |
| 104 | N | N-methyl-N-(naphth- | H | methoxy | 2,6-diethyl- | 451.2 |
| | | 1-yl)-amino- | | | phenyl | |
| 105 | N | naphth-1-yl-oxy- | H | methoxy | 2,6-diethyl- | 438.2 |
| | | | | | phenyl | |
| 106 | N | N-ethyl-N-(naphth- | H | methoxy | 2-(benzo[1,3]- | 453.2 |
| | | 1-yl)-amino- | | | dioxolyl) | |
| 107 | N | N-ethyl-N-(naphth- | H | methoxy | 2-(biphenyl) | 485.3 |
| | | 1-yl)-amino- | | | | |
| 108 | N | N-(naphth-1-yl)- | H | methoxy | 2,6-diethyl- | 481.3 |
| | | amino-ethyl-oxo- | | | phenyl | |
| 109 | N | N-ethyl-N-(naphth- | H | H | 2,6-diethyl- | 435.3 |
| | | 1-yl)-amino- | | | phenyl | |
| 110 | N | N-methyl-N-(2- | H | methoxy | 2,6-dimethyl- | 429.3 |
| | | isopropyl-5-methyl- | | | phenyl | |
| | | phenyl)-amino- | | | | |
| 111 | N | 2-methyl-5-isopropyl- | H | methoxy | 2,6-dimethyl- | 416.2 |
| | | phenyl-oxy- | | | phenyl | |
| 112 | N | N-(methyl)-N-(5-(2- | H | methoxy | 2,6-dimethyl- | 442.3 |
| | | methyl-1,2,3,4- | | | phenyl | |
| | | tetrahydro- | | | | |
| | | isoquinolinyl)-amino- | | | | |
| 113 | N | N-(methyl)-N-(indol- | H | methoxy | 2,6-dimethyl- | 41.2 |
| | | 4-yl)-amino | | | phenyl | |
| 114 | N | N-(methyl)-N-(2,6- | H | methoxy | 2,6-dimethyl- | 401.2 |
| | | dimethyl-phenyl)- | | | phenyl | |
| | | amino- | | | | |
| 115 | N | N-(methyl)-N-(2- | H | methoxy | 2,6-dimethyl- | 421.2 |
| | | methyl-3-chloro- | | | phenyl | |
| | | phenyl)-amino- | | | | |
| 116 | N | N-(methyl)-N-(7- | H | methoxy | 2,6-dimethyl- | 453.1 |
| | | methoxy-naphth-1- | | | phenyl | |
| | | yl)-amino- | | | | |
| 117 | N | N-(methyl)-N-(2- | H | methoxy | 2,6-dimethyl- | 417.1 |
| | | methyl-3-methoxy- | | | phenyl | |
| | | phenyl)-amino- | | | | |
| 118 | N | N-(methyl)-N-(5- | H | methoxy | 2,6-diethyl- | 481.3 |
| | | methoxy-naphth-1- | | | phenyl | |
| | | yl)-amino- | | | | |
| 119 | N | N-(2-methyl-naphth- | H | methoxy | 2,6-diethyl- | 451.2 |
| | | 1-yl)-amino- | | | phenyl | |
| 120 | N | N-(2-trifluoromethyl- | H | methoxy | 2,6-diethyl- | 455.2 |
| | | phenyl)-amino- | | | phenyl | |
| 121 | N | N-(4-indanyl)-amino- | H | methoxy | 2,6-diethyl- | 427.3 |
| | | | | | phenyl | |
| 122 | N | N-(methyl)-N-(3-(4- | H | methoxy | 2,6-diethyl- | 491.3 |
| | | methyl-biphenyl))- | | | phenyl | |
| | | amino- | | | | |
| 123 | N | N-(methyl)-N- | H | methoxy | 2,6-diethyl- | 415.3 |
| | | (benzyl)-amino- | | | phenyl | |
| 124 | N | N-(methyl)-N- | H | methoxy | 2,6-diethyl- | 407.3 |
| | | (cyclohexyl)-amino- | | | phenyl | |
| 125 | N | N-(methyl)-N-(3- | H | methoxy | 2,6-diethyl- | 416.3 |
| | | pyridyl-methyl)- | | | phenyl | |
| | | amino- | | | | |
| 126 | N | N-pyrrolidinyl | H | methoxy | 2,6-diethyl- | 365.2 |
| | | | | | phenyl | |
| 127 | N | 2-(6,7-dimethoxy- | H | methoxy | 2,6-diethyl- | 487.3 |
| | | 1,2,3,4-tetrahydro- | | | phenyl | |
| | | isoquinolinyl) | | | | |
| 128 | N | 2-(6-carboxy-1,2,3,4- | H | methoxy | 2,6-diethyl- | 471.2 |
| | | tetrahydro- | | | phenyl | |
| | | isoquinolinyl) | | | | |
| 129 | N | 2-(5-carboxy-1,2,3,4- | H | methoxy | 2,6-diethyl- | 471.2 |
| | | tetrahydro- | | | phenyl | |
| | | isoquinolinyl) | | | | |
| 130 | N | 2-(8-carboxy-1,2,3,4- | H | methoxy | 2,6-diethyl- | 471.3 |
| | | tetrahydro- | | | phenyl | |
| | | isoquinolinyl) | | | | |
| 131 | N | 2-(7-carboxy-1,2,3,4- | H | methoxy | 2,6-diethyl- | 471.2 |
| | | tetrahydro- | | | phenyl | |
| | | isoquinolinyl) | | | | |
| 132 | N | N-(naphth-1-yl)- | H | methoxy | 2,6-diethyl- | 437.2 |
| | | amino- | | | phenyl | |
| 133 | N | N-(ethyl)-N- | H | methoxy | phenyl | 409.2 |
| | | (naphthyl-1-yl)- | | | | |
| | | amino- | | | | |
| 134 | N | N-(ethyl)-N-(naphth- | H | methoxy | 3-thienyl | 415.2 |
| | | 1-yl)-amino- | | | | |
| 135 | N | N-(ethyl)-N- | H | methoxy | 2,6-diethyl- | 467.3 |
| | | (quinazolin-4-yl)- | | | phenyl | |
| | | amino- | | | | |
| 136 | N | N-(ethyl)-N-(naphth- | H | methoxy | 3-pyridyl | 410.3 |
| | | 1-yl)-amino- | | | | |
| 137 | N | N-(2-(4-methoxy- | H | methoxy | 2,6-diethyl- | 493.3 |
| | | biphenyl))-amino- | | | phenyl | |
| 138 | N | N-(ethyl)-amino- | H | methoxy | 2,6-diethyl- | 339.3 |
| | | | | | phenyl | |
| 139 | N | 2-(hydroxymethyl)- | H | methoxy | 2,6-diethyl- | 418.2 |
| | | phenyl-oxy- | | | phenyl | |
| 140 | N | N-(2-methyl-5- | methyl | methoxy | 2,6-diethyl- | 445.2 |
| | | methoxy-phenyl)- | | | phenyl | |
| | | amino- | | | | |
| 141 | N | N-(2-methyl-4- | H | methoxy | 2,6-diethyl- | 431.2 |
| | | hydroxymethyl- | | | phenyl | |
| | | phenyl)-amino- | | | | |
| 142 | N | N-(3-hydroxymethyl- | H | methoxy | 2,6-diethyl- | 417.2 |
| | | phenyl)-amino- | | | phenyl | |
| 143 | N | 2-formyl-5-methoxy- | H | methoxy | 2,6-diethyl- | 446.2 |
| | | phenyl-oxy- | | | phenyl | |
| 144 | N | N-(2-methoxy- | H | methoxy | 2,6-diethyl- | 445.2 |
| | | carbonyl-phenyl)- | | | phenyl | |
| | | amino- | | | | |
| 145 | N | N-(2-amino-benzyl)- | H | methoxy | 2,6-diethyl- | 416.2 |
| | | amino- | | | phenyl | |
| 146 | N | N-(2-methyl-5- | H | hydroxy | 2,6-diethyl- | 403.2 |
| | | hydroxy-phenyl)- | | | phenyl | |
| | | amino- | | | | |
| 147 | N | N-(2-methylcarbonyl- | H | methoxy | 2,6-diethyl- | 429.2 |
| | | phenyl)-amino- | | | phenyl | |
| 148 | N | N-(2-methyl-5- | H | methoxy | 2,6-diethyl- | 417.2 |
| | | hydroxy-phenyl)- | | | phenyl | |
| | | amino | | | | |
| 149 | N | N-(2-aminocarbonyl- | H | methoxy | 2,6-diethyl- | 430.1 |
| | | phenyl)-amino- | | | phenyl | |
| 150 | N | 2-ethoxycarbonyl- | H | methoxy | 2,6-diethyl- | 460.1 |
| | | phenyl-oxy- | | | phenyl | |
| 151 | N | N-(ethyl)-N-(naphth- | H | methoxy | 4-(3,5-dimethyl- | 428.2 |
| | | 1-yl)-amino- | | | isoxazolyl) | |
| 152 | N | N-(ethyl)-N-(naphth- | H | methoxy | 2,6-diethyl- | ^{a} |
| | | 1-yl)-amino- | | | phenyl | |

^{a} Compounds #4 and #152 are the separated enantiomers of compound #12, and their individual MS values are not listed.

Representative compounds of the present invention were tested in in vitro assays (functional and binding) according to the procedures as described in Examples 9 and 10, below; with results as listed in Table 3 which follows herein.

### Example 9

### Calcium Mobilization Assay

U937 cells (human monocytic cell line) were cultured in RPMI-1640 medium supplemented with 10% fetal bovine serum, 10 mM HEPES, 0.11 g/L pyruvate, 0.29 g/L L-glutamine, 100 IU/mL penicillin G and 0.1 g/mL streptomycin. Dibutyryl cAMP (1 mM) was added to induce differentiation 2 or 3 days prior to loading the cells with 4.4 μM Fluo-3AM (Molecular Probes) for 30 min at 37° C. in RPMI medium containing 20 mM HEPES, 0.1% bovine serum albumin (BSA) and 5 mM probenecid. After washing, cells were dissolved at 2×10⁶ cells/ml in HBSS buffer (Invitrogen 14190-094) supplemented with 10 mM HEPES, 2.5 mM probenecid and 0.1% BSA (pH=7.4), and sedimented by 1 min centrifugation (1500 rpm) in black 96-well plates (Costar) (200,000 cells/well). A 100× concentration range of test compounds was prepared in DMSO and further diluted in HBSS buffer. U937 cells were pre-incubated with compound for 20 minutes at room temperature (1% DMSO), before recombinant human C5a (Sigma) was added to the cells (1.5 nM). Changes in intracellular free Ca²⁺ concentration were measured using the Fluorescent Imaging Plate Reader (FLIPR, Molecular Devices). Fluorescence was recorded every second from 10 seconds before the addition of C5a until 2 min after the addition. The maximal fluorescence obtained during this time frame was used for further calculations. The IC₅₀ value was calculated as the molar concentration of the test compound, which inhibits 50% of specific C5a-induced Ca²⁺ mobilization.

### Example 10

### [¹²⁵I]-C5a Radioligand Binding Assay

Competition binding assays for C5a receptors were performed by incubation of differentiated U937 cells (200,000 cells/well), test compound (1% DMSO) and [¹²⁵I]-C5a (Bolton & Hunter labeled, Perkin Elmer, specific activity=2200 Ci/mmol, 0.05 nM final) in binding buffer consisting of 50 mM HEPES, 5 mM MgCl₂, 1 mM CaCl₂, 0.5% protease-free BSA, 0.02% NaN₃, pH 7.4. 100× solutions of test compounds were prepared in DMSO and further diluted in binding buffer. Non-specific binding was defined in wells containing 100 nM recombinant C5a. Cells and compound were pre-incubated for 30 min at RT. After addition of the radioligand, further incubation for 60 min occurred at 4° C. Cells were harvested on GF/B filters—presoaked in 0.5% polyethylenimine, followed by washing with binding buffer containing 500 mM NaCl, pH 7.4. Filter bound radioactivity was determined by liquid scintillation counting. IC₅₀ and Kᵢ values were calculated using non-linear regression in Graphpad Prism. Kᵢ is calculated using the equation of Cheng and Prusoff (Biochem. Pharmacol. 1973, 22: 3099-3108)

$${K}_{i} = \frac{{IC}_{50}}{1 + \frac{[ radioligand ]}{{K}_{d}}} .$$

Representative compounds of the present invention were tested according to the procedures as described in Example 9 and 10 above, with results as listed in Table 3, below. Unless otherwise noted, multiple values in a cell below recite the results of duplicate experiments. ([¹²⁵I]-C5a=0.05 nM; C5a K_{d}=0.324 nM.)

TABLE 3
In vivo Results

| | C5a Induced | | | |
|---|---|---|---|---|
| | Ca Mobil. | [¹²⁵I]-C5a Binding | | [¹²⁵I]-C5a |
| | Average IC₅₀ | % Inhibition | | Binding |
| ID No. | (μM) | 10 μM | 1 μM | Ki (μM)^{a} |
| 1 | 0.0073 | | | |
| 2 | 0.022 | | | |
| 3 | 0.026 | | | |
| 4 | 0.027 | 95, 101 | 94, 99 | |
| 5 | 0.030 | 97 | 92 | |
| 6 | 0.037 | | | |
| 7 | 0.053 | | | |
| 8 | 0.067 | 96 | 94 | |
| 9 | 0.071 | 97 | 91 | 0.013 |
| 10 | 0.082 | 94 | 92 | |
| 11 | 0.092, 0.173 | 92, 97 | 88, 96 | 0.010 |
| 12 | 0.094 | 97, 92, | 94, 88, 97, | 0.010, 0.0078 |
| | | 102, 102, | 98, 101, | |
| | | 90 | 100, 81 | |
| 13 | 0.109 | | | |
| 14 | 0.117, 0.222 | 93, 95 | 82, 90 | 0.184 |
| 15 | 0.177 | 90 | 79 | |
| 16 | 0.133 | 90 | 88 | |
| 17 | 0.134 | | | |
| 18 | 0.137 | | | |
| 19 | 0.161 | 86, 98 | 81, 82 | 0.106 |
| 20 | 0.165 | 93, 102, | 84, 99, 99 | 0.0073 |
| | | 100 | | |
| 21 | 0.169 | 93 | 91 | |
| 22 | 0.176 | 86 | 87 | |
| 23 | 0.191 | | | |
| 24 | 0.216 | 91 | 88 | |
| 25 | 0.244 | | | |
| 26 | 0.266 | 94, 99 | 93, 96 | 0.0097 |
| 27 | 0.273 | 94 | 90 | |
| 28 | 0.287 | 92 | 88 | |
| 29 | 0.338 | 89 | 88 | |
| 30 | 0.354 | 82 | 68 | |
| 31 | 0.373, 1.42 | 87, 101, | 77, 94, 96, | 0.035 |
| | | 100, 83 | 69 | |
| 32 | 0.380 | 91 | 88 | |
| 33 | 0.414 | | | |
| 34 | 0.417 | 89 | 83 | |
| 35 | 0.426 | 89 | 81 | |
| 36 | 0.531 | 87 | 86 | |
| 37 | 0.543 | 86 | 74 | |
| 38 | 0.560 | 87 | 84 | |
| 39 | 0.647 | 87, 98 | 81, 90 | 0.086 |
| 40 | 0.687 | 88 | 88 | |
| 41 | 0.732 | 86 | 85 | |
| 42 | 0.792 | 86 | 83 | |
| 43 | 0.817 | 83 | 80 | |
| 44 | 0.839 | 87 | 77 | |
| 45 | 0.878 | 87 | 74 | |
| 46 | 0.910, 3.24 | 87 | 81 | |
| 47 | 0.949 | 89 | 83 | |
| 48 | 1.21 | 91 | 85 | |
| 49 | 1.38 | 80 | 59 | |
| 50 | 1.38 | 85 | 83 | |
| 51 | 1.41 | 89 | 83 | |
| 52 | 1.48 | 86 | 86 | |
| 53 | 1.49 | 91 | 85 | |
| 54 | 1.58 | 87 | 74 | |
| 55 | 1.65 | 61 | 24 | |
| 56 | 1.69 | 85 | 84 | |
| 57 | 1.73 | 89 | 76 | |
| 58 | 1.77 | 83, 88 | 59, 46 | 1.16 |
| 59 | 1.90 | 89 | 91 | 0.057 |
| 60 | 1.97 | 83, 85 | 39, 79 | 0.179 |
| 61 | 2.19 | 88 | 76 | |
| 62 | 2.32 | | | |
| 63 | 2.35 | 82 | 74 | |
| 64 | 2.39 | | | |
| 65 | 2.43 | 32, 10 | −7, 1 | |
| 66 | 2.46 | 88 | 83 | |
| 67 | 2.56 | 86 | 82 | |
| 68 | 2.57 | 76 | 66 | |
| 69 | 3.22 | 85 | 76 | |
| 70 | 3.47 | | | |
| 71 | 3.47 | | | |
| 72 | 3.79 | 85 | 86 | |
| 73 | 4.59 | 80, 70 | 55, 34 | 3.69 |
| 74 | 4.90 | 52 | 19 | |
| 75 | 4.96 | | | |
| 76 | 5.13 | 85 | 83 | |
| 77 | 5.50 | 72 | 555 | |
| 78 | 5.86 | 85, 95 | 71, 72 | 0.368 |
| 79 | 6.17 | 78 | 49 | |
| 80 | 6.19 | 81 | 80 | |
| 81 | 7.41 | 71 | 55 | |
| 82 | 7.94 | 81, 76 | 66, 55 | 0.678 |
| 83 | 8.13 | 71 | 51 | |
| 85 | >10 | 75, 79 | 56, 49 | 0.963 |
| 86 | >10 | 64 | 49 | |
| 87 | >10 | 51 | 17 | |
| 88 | >10 | 51 | 25 | |
| 89 | >10 | 75, 77 | 71, 37 | 1.70 |
| 90 | >10 | 86, 84 | 87, 71 | 0.409 |
| 91 | >10 | 60 | 20 | |
| 92 | >10 | 62 | 24 | |
| 93 | >10 | 80 | 68 | |
| 94 | >10 | 77 | 56 | |
| 95 | >10 | 68, 80 | 45, 24 | 2.06 |
| 96 | >10 | 87 | 77 | |
| 97 | >10 | 59 | 47 | |
| 98 | >10 | 70 | 43 | |
| 99 | >10 | 76 | 59 | |
| 100 | >10 | 82 | 70 | |
| 101 | >10 | 79 | 75 | |
| 102 | >10 | 80 | 74 | |
| 103 | >10 | 61 | 48 | |
| 104 | >10 | 64 | 56 | |
| 105 | >10 | 70 | 55 | |
| 106 | >10 | 68 | 39 | |
| 107 | >10 | 84 | 76 | |
| 108 | >10 | 84 | 54 | |
| 109 | >10 | 83, 78 | 65, 43 | 1.06 |
| 110 | >10 | 82 | 77 | |
| 111 | >10 | 85 | 82 | |
| 112 | >10 | 70 | 36 | |
| 113 | >10 | 54 | 33 | |
| 114 | >10 | 73 | 63 | |
| 115 | >10 | 80 | 75 | |
| 116 | >10 | 86 | 87 | |
| 117 | >10 | 85 | 86 | |
| 118 | >10 | 77 | 71 | |
| 119 | >10 | 74 | 64 | |
| 120 | >10 | 82 | 75 | |
| 121 | >10 | 75 | 59 | |
| 122 | >10 | 84 | 81 | |
| 123 | >10 | 39 | 3 | |
| 124 | >10 | 33 | 2 | |
| 125 | >10 | 45 | −11 | |
| 126 | >10 | 18 | −11 | |
| 127 | >10 | 42 | −9 | |
| 128 | >10 | −9 | −25 | |
| 129 | >10 | −20 | −46 | |
| 130 | >10 | −13 | −24 | |
| 131 | >10 | −9 | −26 | |
| 132 | >10 | 34 | 12 | |
| 133 | >10 | 6, 53 | −15, 14 | 8.29 |
| 134 | >10 | 40 | −8 | |
| 135 | >10 | 47 | 16 | |
| 136 | >10 | 35 | 2 | |
| 137 | >10 | | | |
| 138 | >10 | | | |
| 139 | >10 | | | |
| 140 | >10 | | | |
| 141 | >10 | | | |
| 142 | >10 | | | |
| 143 | >10 | | | |
| 144 | >10 | | | |
| 145 | >10 | | | |
| 146 | >10 | | | |
| 147 | >10 | | | |
| 148 | >10 | | | |
| 149 | >10 | | | |
| 150 | >10 | | | |
| 151 | >10 | | | |
| 152 | >10 | | | |

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A compound of formula (I): 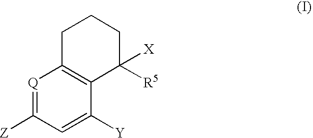
wherein:
X is —NR¹R², —OR³, or —SR³;
Y is —OR⁷, —SR⁷, R⁸, halo, or hydrogen;
Z is C₆₋₁₅ aryl optionally substituted with 1-3 R⁴ or a 5 to 15 membered heteroaryl optionally substituted with 1-3 R⁴;
R¹ is C₁₋₁₀ alkyl optionally substituted with 1-3 R⁴, C₃₋₁₀ cycloalkyl optionally substituted with 1-3 R⁴, C₆₋₁₅ aryl optionally substituted with 1-3 R⁴, C₆₋₁₅arylC₁₋₆alkyl wherein said aryl group is optionally substituted with 1-3 R⁴, a 5 to 15 membered heteroaryl optionally substituted with 1-3 R⁴, a 5 to 15 membered heteroarylC₁₋₆alkyl wherein said heteroaryl group is optionally substituted with 1-3 R⁴, or a 5 to 15 membered heterocyclyl optionally substituted with 1-3 R⁴;
R² is hydrogen, —C(O)R⁸, or C₁₋₆alkyl optionally substituted with 1-2 groups independently selected from the group consisting of hydroxyl, cyano, and halo;
or R¹ and R² are taken together with the nitrogen atom to which they are attached to form a five to seven membered heterocyclic ring which is optionally fused to C₅₋₆aryl or a C₅₋₇ cycloalkyl wherein said C₅₋₆aryl or C₅₋₇ cycloalkyl is optionally substituted with 1-3 groups independently selected from the group consisting of halo, cyano, C₁₋₃alkyl, C₁₋₃ alkoxy, halogenatedC₁₋₃alkyl, —C(O)R⁶ and —C(O)OR⁶;
R³ is C₆₋₁₅ aryl optionally substituted with 1-3 R⁴, C₆₋₁₅arylC₁₋₆alkyl wherein said aryl group is optionally substituted with 1-3 R⁴, C₆₋₁₅aryl-NH—C₁₋₆alkyl wherein said aryl group is optionally substituted with 1-3 R⁴, a 5 to 15 membered heteroaryl optionally substituted with 1-3 R⁴, or a 5 to 15 membered heteroarylC₁₋₆alkyl wherein said heteroaryl group is optionally substituted with 1-3 R⁴;
R⁴ is hydroxy, halo, cyano, C₁₋₁₀ alkoxy, C₁₋₁₀ alkyl, halogenatedC₁₋₁₀alkyl, C₃₋₁₀ cycloalkyl, aminoC₁₋₁₀alkyl, C₁₋₁₀alkylamino, di(C₁₋₁₀)alkylamino, a 5 to 10 membered heterocyclyl, C₆₋₁₀aryl, a 5 to 10 membered heteroaryl, —NH₂, —C(O)R⁶, —C(O)OR⁶, or C₁₋₁₀ alkylthio, wherein said C₁₋₁₀ alkoxy, C₁₋₁₀alkyl, C₃₋₁₀ cycloalkyl, 5 to 10 membered heterocyclyl, C₆₋₁₀ aryl, 5 to 10 membered heteroaryl, is optionally substituted with hydroxyl, halogenatedC₁₋₃alkyl, cyano, halo, C₁₋₃ alkyl, C₁₋₃ alkoxy, —C(O)R⁶ or —C(O)OR⁶;
R⁵ is hydrogen or C₁₋₆ alkyl;
R⁶ is hydrogen, C₁₋₃ alkyl, or —NH₂;
R⁷ is C₁₋₁₀ alkyl optionally substituted with 1-3 R⁴, or C₃₋₁₀ cycloalkyl optionally substituted with 1-3 R⁴;
R⁸ is C₁₋₆ alkyl, or C₃₋₇ cycloalkyl, and
Q is N or N-oxide;
or enantiomers, stereoisomers, pro-drugs, solvates, or pharmaceutically acceptable salts thereof.

2. A compound according to claim 1, wherein X is NR¹R².

3. A compound according to claim 2, wherein R¹ is selected from the group consisting of C₆₋₁₀aryl, C₆₋₁₀arylC₁₋₃alkyl, a 5 to 10 membered heteroaryl, a 5 to 10 membered heteroarylC₁₋₃alkyl, and a 5 to 10 membered heterocyclyl, wherein each R₁ is optionally substituted 1-3 groups independently selected from C₁₋₆alkyl, halogenatedC₁₋₆alkyl, hydroxyl substituted C₁₋₆alkyl, halo, hydroxyl, C₁₋₆alkoxy, cyano, —NH₂, NH₂C₁₋₃alkyl, —C(O)OR⁶, and —C(O)R⁶.

4. A compound according to claim 2, wherein R² is selected from the group consisting of hydrogen, —C(O)C₁₋₃alkyl, and C₁₋₃alkyl optionally substituted 1-2 groups independently selected from the group consisting of hydroxyl, cyano, and halo.

5. A compound according to claim 2, wherein R² is selected from hydrogen and C₁₋₃alkyl optionally substituted with hydroxyl or cyano.

6. A compound according to claim 2, wherein R¹ is selected from the group consisting of naphthyl, phenyl, naphthylenylmethyl, furanylmethyl, indanyl, phenylpropyl, phenethyl, benzyl, thiophenylmethyl, indolyl, tetrahydroisoquinolyl, tetrahydronaphthyl, pyridyl, and 1,2,3,4-tetrahydroquinolyl, each of which is optionally substituted with 1-3 R⁴.

7. A compound according to claim 6, wherein R² is selected from hydrogen, C₁₋₃alkyl, —CH₂CH₂OH, and —CH₂CH₂CH₂OH.

8. A compound according to claim 1, wherein X is isoindolinyl, indolinyl, tetrahydroisoquinolyl, or 1,2,3,4-tetrahydroquinolyl each of which is optionally substituted 1-2 members independently selected from C₁₋₃alkyl, halogenatedC₁₋₃alkyl, C₁₋₃alkoxy, —C(O)OR⁶, —C(O)R⁶, halo, and cyano.

9. A compound according to claim 1 wherein X is OR³.

10. A compound according to claim 9, wherein R³ is selected from C₆₋₁₀ aryl, C₆₋₁₀arylC₁₋₃alkyl, and C₆₋₁₀aryl-NH—C₁₋₃alkyl, wherein each C₆₋₁₀aryl is optionally substituted with 1-2 groups independently selected from C₁₋₃alkyl, hydroxyl substituted C₁₋₃alkyl, C₁₋₃alkoxy, —C(O)OR⁶, and —C(O)R⁶.

11. A compound according to claim 1, wherein Y is C₁₋₁₀ alkoxy, C₃₋₁₀cycloalkoxy, C₁₋₆ alkyl, C₁₋₁₀alkylthio, halo, phenylC₁₋₃alkoxy, or hydrogen.

12. A compound according to claim 1, wherein Y is C₁₋₃alkoxy, C₁₋₃alkyl, C₁₋₃alkylthio, or C₃₋₇cycloalkoxy, more preferably C₁₋₃ alkoxy or C₁₋₃alkylthio.

13. A compound according to claim 1, wherein Z is C₆₋₁₀aryl optionally substituted with 1-3 R⁴ or a 5 to 10 membered heteroaryl optionally substituted with 1-3 R⁴.

14. A compound according to claim 1, wherein Z is phenyl optionally substituted with 1-3 members independently selected from C₁₋₃alkyl, C₁₋₃alkoxy, and halo.

15. A compound according to claim 1, wherein Z is phenyl substituted at the 2 position relative to the point of attachment.

16. A compound according to claim 1, wherein Z is phenyl substituted at the 2 and 6 position relative to the point of attachment.

17. A compound according to claim 1, wherein R₅ is —CH₃, —CH₂CH₃, or —CH₂CH₂CH₃.

18. A compound according to claim 1, wherein R₅ is H.

19. A compound according to claim 1, wherein Q is N.

20. The compound according to claim 1, wherein
Q is selected from the group consisting of N and N⁺O⁻;
X is selected form the group consisting of (R)—N-ethyl-N-(napthy-1-yl)-amino-, (R)—N-methyl-N—(R)-(1,2,3,4-tetrahydro-naphth-1-yl)-amino-, (R)—N-methyl-N—(S)-1,2,3,4-tetrahydronaphth-1-yl)-amino-, (S)—N-methyl-N—(R)-(1,2,3,4-tetrahydro-naphth-1-yl)-amino-, (S)—N-methyl-N—(S)-(1,2,3,4-tetrahydro-naphth-1-yl)-amino-, 1-(1,2,3,4-tetrahydro-quinolinyl), 2-(1,2,3,4-tetrahydro-isoquinolinyl), 2-(5-chloro-1,2,3,4-tetrahydro-isoquinolinyl), 2-(7-chloro-1,2,3,4-tetrahydro-isoquinolinyl), 2-(7-fluoro-1,2,3,4-tetrahydro-isoquinolinyl)-, 2-(8-methoxycarbonyl-1,2,3,4-tetrahydro-isoquinolinyl)-, 2-(8-trifluoromethyl-1,2,3,4-tetrahydroisoquinolinyl), 2-hydroxyethyl-phenyl-oxy-, 2-methyl-5-methoxy-phenyl-oxy-, N-(2,5-dimethoxy-phenyl)-amino-, N-(2,5-dimethyl-phenyl)-amino-, N-(2-aminomethyl-phenyl)-amino-, N-(2-carboxyphenyl)-amino-, N-(2-chloro-5-methoxy-phenyl)-amino-, N-(2-cyano-phenyl-amino-, N-(2-ethyl-phenyl)-amino-, N-(2-hydroxyethyl-phenyl)-amino-, N-(2-hydroxymethyl-4-chloro-phenyl)-amino-, N-(2-hydroxymethyl-4-fluoro-phenyl)-amino-, N-(2-hydroxymethyl-5-chloro-phenyl)-amino, N-(2-hydroxymethyl-phenyl)-amino-, N-(2-methyl-4-methoxy-phenyl)-amino-, N-(2-methyl-5-chloro-phenyl)-amino-, N-(2-methyl-5-fluoro-phenyl)-amino-, N-(2-methyl-5-methoxy-phenyl)-amino-, N-(2-methyl-5-phenyl-phenyl)-amino-, N-(2-methyl-5-trifluoromethyl-phenyl)-amino-, N-(2-methylphenyl)-amino-, N-(2-hydroxymethyl-5-trifluoromethyl-phenyl )-amino-, N-(3-methoxy-phenyl)-amino-, N-(4-hydroxymethyl-pyrid-3-yl)-amino, N-(cyanomethyl)-N-(naphth-1-y)-amino-, N-(hydroxyethyl)-N-(naphth-1-yl)-amino-, N-(methylcarbonyl)-N-(naphth-1-yl)-amino-, N-ethyl-N-(2,3-dimethyl-phenyl)-amino-, N-ethyl-N-(5,6,7,8-tetrahydro-naphth-1-yl)-amino-, N-(ethyl)-N-(naphth-1-yl)-amino-, N-methyl-(2,5-dimethoxy-phenyl)-amino-, N-methyl-N-(1,2,3,4-tetrahydroquinolin-1-yl), N-methyl-N-(2,5-dimethyl-phenyl)-amino-, N-methyl-N-(2-chloro-5-methoxy-phenyl)-amino-, N-methyl-N-(2-methyl-4-methoxy-phenyl)-amino-, N-methyl-N-(2-methyl-5-chloro-phenyl)-amino-, N-methyl-N-(2-methyl-5-methoxy-phenyl)-amino-, N-methyl-N-(2-methyl-naphth-1-yl)-amino-, N-methyl-N-(2-methylphenyl)-amino-, N-methyl-N-(4-chloro-naphth-1-yl)-amino-, N-methyl-N-(4-methoxy-naphth-1-yl)-amino-, N-methyl-N-(6-methoxy-naphth-1-yl)-amino-, N-methyl-N-(indan-4-yl), N-methyl-N-(naphth-1-yl)-amino- and N-methyl-N-phenyl-amino-;
R⁵ is selected from the group consisting of hydrogen and n-propyl;
Y is selected from the group consisting of chloro, ethyl, methoxy, ethoxy, isopropoxy, methylthio, benzyloxy, cyclopentyl-oxy and dimethylamino-ethoxy;
Z is selected from the group consisting of 2-fluorophenyl, 2-methylphenyl, 2-ethylphenyl, 2-isopropylphenyl, 2.6-diethylphenyl, 2,6-dimethyl-4-methoxy-phenyl, 2-chloro-6-methoxy-phenyl and naphth-1-yl;
or an enantiomers, stereoisomer, pro-drug, solvate, or pharmaceutically acceptable salt thereof.

21. A compound according to claim 1, wherein Q, X, R⁵, Y and Z are selected from the group consisting of

| Q | X | R⁵ | Y | Z |
|---|---|---|---|---|
| N | N-(2-hydroxy-ethyl-phenyl)- | H | isopropoxy | 2,6-diethyl- |
| | amino- | | | phenyl |
| N | N-(2-hydroxymethyl-5- | H | methoxy | 2,6-diethyl-phenyl |
| | trifluoromethyl-phenyl)-amino- | | | |
| N | N-(2-hydroxymethyl-5-chloro- | H | methoxy | 2,6-diethyl-phenyl |
| | phenyl)-amino | | | |
| N | (R)—N-ethyl-N-(napthy-1-yl)- | H | methoxy | 2,6-diethyl-phenyl |
| | amino- | | | |
| N | N-(2-hydroxyethyl-phenyl)- | H | methoxy | 2,6-diethyl-phenyl |
| | amino- | | | |
| N | N-ethyl-N-(naphth-1-yl)-amino- | H | cyclopentyloxy | 2,6-diethyl-phenyl |
| N | N-(2-hydroxymethyl-4-fluoro- | H | methoxy | 2,6-diethyl-phenyl |
| | phenyl)-amino- | | | |
| N | N-ethyl-N-(naphth-1-yl)-amino- | H | ethoxy | 2,6-diethyl-phenyl |
| N | N-(2-hydroxymethyl-4-chloro- | H | methoxy | 2,6-diethyl-phenyl |
| | phenyl)-amino- | | | |
| N | N-ethyl-N-(naphth-1-yl)-amino- | H | methylthio | 2,6-diethyl-phenyl |
| N | N-(2-methyl-5-methoxy- | H | methoxy | 2,6-diethyl-phenyl |
| | phenyl)-amino- | | | |
| N | N-ethyl-N-(naphth-1-yl)-amino- | H | methoxy | 2,6-diethyl-phenyl |
| N | N-ethyl-N-(naphth-1-yl)-amino- | H | methoxy | 2-chloro-6- |
| | | | | methoxy- |
| | | | | phenyl |
| N | N-(hydroxyethyl)-N-(naphth-1- | H | methoxy | 2,6-diethyl- |
| | yl)-amino- | | | phenyl |
| N | N-(2-hydroxymethyl-phenyl)- | H | methoxy | 2,6-diethyl- |
| | amino- | | | phenyl |
| N | N-(ethyl)-N-(naphth-1-yl)- | H | methoxy | 2,6-dimethyl- |
| | amino- | | | phenyl |
| N | N-(2-methyl-5-methoxy- | H | cyclo-pentyloxy | 2,6-diethyl- |
| | phenyl)-amino- | | | phenyl |
| N | N-(2-methyl-5-methoxy- | H | iso-propoxy | 2,6-diethyl- |
| | phenyl)-amino- | | | phenyl |
| N⁺O⁻ | N-ethyl-N-(naphth-1-yl)-amino- | H | methoxy | 2,6-diethyl- |
| | | | | phenyl |
| N | N-methyl-N-(naphth-1-yl)- | H | methoxy | 2,6-diethyl- |
| | amino- | | | phenyl |
| N | N-ethyl-N-(naphth-1-yl)-amino- | H | methoxy | 2,6-dimethyl-4- |
| | | | | methoxy- |
| | | | | phenyl |
| N | N-methyl-N-(6-methoxy- | H | methoxy | 2,6-diethyl- |
| | naphth-1-yl)-amino- | | | phenyl |
| N | N-ethyl-N-(naphth-1-yl)-amino- | H | ethyl | 2,6-diethyl- |
| | | | | phenyl |
| N | N-ethyl-N-(5,6,7,8-tetrahydro- | H | methoxy | 2,6-diethyl- |
| | naphth-1-yl)-amino- | | | phenyl |
| N | N-(2-methyl-5-methoxy- | H | ethyl | 2,6-diethyl- |
| | phenyl)-amino- | | | phenyl |
| N | 2-(8-trifluoromethyl-1,2,3,4- | H | methoxy | 2,6-diethyl- |
| | tetrahydroisoquinolinyl) | | | phenyl |
| N | N-ethyl-N-(naphth-1-yl)-amino- | H | benzyloxy | 2,6-diethyl- |
| | | | | phenyl |
| N | N-(2-methyl-5-trifluoromethyl- | H | methoxy | 2,6-diethyl- |
| | phenyl)-amino- | | | phenyl |
| N | 2-methyl-5-methoxy-phenyl- | H | methoxy | 2,6-diethyl- |
| | oxy- | | | phenyl |
| N | N-ethyl-N-(naphth-1-yl)-amino- | H | methoxy | 2-methyl- |
| | | | | phenyl |
| N | 2-(1,2,3,4- | H | methoxy | 2,6-diethyl- |
| | tetrahydroisoquinolinyl) | | | phenyl |
| N | N-(2-chloro-5-methoxy- | H | methoxy | 2,6-diethyl- |
| | phenyl)-amino- | | | phenyl |
| N | N-(2-carboxyphenyl)-amino- | H | methoxy | 2,6-diethyl- |
| | | | | phenyl |
| N | N-(2,5-dimethoxy-phenyl)- | H | methoxy | 2,6-diethyl- |
| | amino- | | | phenyl |
| N | N-(2,5-dimethylphenyl)-amino- | H | methoxy | 2,6-diethyl- |
| | | | | phenyl |
| N | N-methyl-N-(4-methoxy- | H | methoxy | 2,6-diethyl- |
| | naphth-1-yl)-amino- | | | phenyl |
| N | N-(2-methyl-5-methoxy- | H | methoxy | 2,6-dimethyl- |
| | phenyl)-amino- | | | phenyl |
| N | N-methyl-N-(2-methyl-5- | H | methoxy | 2,6-diethyl- |
| | methoxy-phenyl)-amino- | | | phenyl |
| N | N-ethyl-N-(naphth-1-yl)-amino- | H | methoxy | 2-ethyl-phenyl |
| N | N-methyl-N-(2,5-dimethyl- | H | methoxy | 2,6-diethyl- |
| | phenyl)-amino- | | | phenyl |
| N | N-(2-ethyl-phenyl)-amino- | H | methoxy | 2,6-diethyl- |
| | | | | phenyl |
| N | N-methyl-N-(2-methylphenyl)- | H | methoxy | 2,6-diethyl- |
| | amino- | | | phenyl |
| N | (R)—N-methyl-N—(S)-1,2,3,4- | H | methoxy | 2,6-diethyl- |
| | tetrahydronaphth-1-yl)-amino- | | | phenyl |
| N | 2-(8-methoxycarbonyl-1,2,3,4- | H | methoxy | 2,6-diethyl- |
| | tetrahydro-isoquinolinyl)- | | | phenyl |
| N | 2-(7-fluoro-1,2,3,4-tetrahydro- | H | methoxy | 2,6-diethyl- |
| | isoquinolinyl)- | | | phenyl |
| N | N-(2-methylphenyl)-amino- | H | methoxy | 2,6-diethyl- |
| | | | | phenyl |
| N | N-methyl-N-(2-chloro-5- | H | methoxy | 2,6-diethyl- |
| | methoxy-phenyl)-amino- | | | phenyl |
| N | N-methyl-N-(4-chloro-naphth- | H | methoxy | 2,6-dimethyl- |
| | 1-yl)-amino- | | | phenyl |
| N | N-(cyanomethyl)-N-(naphth-1- | H | methoxy | 2,6-dimethyl- |
| | y)-amino- | | | phenyl |
| N | N-(2-methyl-5-fluoro-phenyl)- | H | methoxy | 2,6-diethyl- |
| | amino- | | | phenyl |
| N | 2-(7-chloro-1,2,3,4-tetrahydro- | H | methoxy | 2,6-diethyl- |
| | isoquinolinyl) | | | phenyl |
| N | N-methyl-N-(2-methyl-naphth- | H | methoxy | 2,6-diethyl- |
| | 1-yl)-amino- | | | phenyl |
| N | N-(2-methyl-5-chloro-phenyl)- | H | methoxy | 2,6-diethyl- |
| | amino- | | | phenyl |
| N | N-methyl-N-(1,2,3,4- | H | methoxy | 2,6-diethyl- |
| | tetrahydroquinolin-1-yl) | | | phenyl |
| N | N-(hydroxyethyl)-N-(naphth-1- | H | methoxy | 2,6-dimethyl- |
| | yl)-amino- | | | phenyl |
| N | 2-(5-chloro-1,2,3,4- | H | methoxy | 2,6-diethyl- |
| | tetrahydroisoquinolinyl) | | | phenyl |
| N | N-ethyl-N-(naphth-1-yl)-amino- | H | methoxy | 2-isopropyl- |
| | | | | phenyl |
| N | N-ethyl-N-(naphth-1-yl)-amino- | H | methoxy | 2,6-dimethoxy- |
| | | | | phenyl |
| N | (S)—N-methyl-N—(R)-(1,2,3,4- | H | methoxy | 2,6-diethyl- |
| | tetrahydro-naphth-1-yl)-amino- | | | phenyl |
| N | N-ethyl-N-(naphth-1-yl)-amino- | H | chloro | 2,6-diethyl- |
| | | | | phenyl |
| N | N-methyl-N-(naphth-1-yl)- | H | methoxy | 2,6-dimethyl- |
| | amino- | | | phenyl |
| N | N-(2-methyl-5-methoxy- | H | dimethyl- | 2,6-diethyl- |
| | phenyl)-amino- | | amino-ethoxy | phenyl |
| N | N-(2-methyl-4-methoxy- | H | methoxy | 2,6-diethyl- |
| | phenyl)-amino- | | | phenyl |
| N | 2-hydroxyethyl-phenyl-oxy- | H | methoxy | 2,6-diethyl- |
| | | | | phenyl |
| N | 1-(1,2,3,4-tetrahydro- | H | methoxy | 2,6-diethyl- |
| | quinolinyl) | | | phenyl |
| N | N-(2-methyl-5-phenyl-phenyl)- | H | methoxy | 2,6-diethyl- |
| | amino- | | | phenyl |
| N | N-methyl-N-(2-methyl-5-chloro- | H | methoxy | 2,6-diethyl- |
| | phenyl)-amino- | | | phenyl |
| N | N-methyl-(2,5-dimethoxy- | H | methoxy | 2,6-diethyl- |
| | phenyl)-amino- | | | phenyl |
| N | N-methyl-N-(2-methyl-5- | H | methoxy | 2,6-dimethyl- |
| | methoxy-phenyl)-amino- | | | phenyl |
| N | N-(2-methyl-5-methoxy- | n-propyl | methoxy | 2,6-diethyl- |
| | phenyl)-amino- | | | phenyl |
| N | N-(4-hydroxymethyl-pyrid-3-yl)- | H | methoxy | 2,6-diethyl- |
| | amino | | | phenyl |
| N | N-methyl-N-(indan-4-yl) | H | methoxy | 2,6-diethyl- |
| | | | | phenyl |
| N | N-ethyl-N-(naphth-1-yl)-amino- | H | methoxy | 2-fluoro-phenyl |
| N | N-(2-cyano-phenyl-amino- | H | methoxy | 2,6-diethyl- |
| | | | | phenyl |
| N | N-(2-aminomethyl-phenyl)- | H | methoxy | 2,6-diethyl- |
| | amino- | | | phenyl |
| N | N-ethyl-N-(2,3-dimethyl- | H | methoxy | 2,6-dimethyl- |
| | phenyl)-amino- | | | phenyl |
| N | (S)—N-methyl-N—(S)-(1,2,3,4- | H | methoxy | 2,6-diethyl- |
| | tetrahydro-naphth-1-yl)-amino- | | | phenyl |
| N | N-ethyl-N-(naphth-1-yl)-amino- | H | methoxy | naphth-1-yl |
| N | N-(methylcarbonyl)-N-(naphth- | H | methoxy | 2,6-diethyl- |
| | 1-yl)-amino- | | | phenyl |
| N | N-methyl-N-(2-methyl-4- | H | methoxy | 2,6-diethyl- |
| | methoxy-phenyl)-amino- | | | phenyl |
| N | N-(3-methoxy-phenyl)-amino- | H | methoxy | 2,6-diethyl- |
| | | | | phenyl |
| N | N-methyl-N-phenyl-amino- | H | methoxy | 2,6-diethyl- |
| | | | | phenyl |
| N | (R)—N-methyl-N—(R)-(1,2,3,4- | H | methoxy | 2,6-diethyl- |
| | tetrahydro-naphth-1-yl)-amino- | | | phenyl |

24. A compound as in claim 1, wherein X is other than 2-(5-carboxy-1,2,3,4-tetrahydro-isoquinolinyl), 2-(6-carboxy-1,2,3,4-tetrahydro-isoquinolinyl), 2-(7-carboxy-1,2,3,4-tetrahydro-isoquinolinyl) or 2-(8-carboxy-1,2,3,4-tetrahydro-isoquinolinyl).

25. A compound according to claim 1 wherein
Q is selected from the group consisting of N;
X is selected from the group consisting of —NR¹R², —OR³, and —SR³;
Y is selected from the group consisting of —OR⁷, —SR⁷, R⁸, halo and hydrogen;
Z is selected from the group consisting of C₆₋₁₅ aryl and a 5 to 15 membered heteroaryl; wherein the C₆₋₁₅aryl or 5 to 15 membered heteroaryl is optionally substituted with 1 to 3 R⁴ substituents;
R⁵ is hydrogen or C₁₋₆ alkyl;
R¹ is selected from the group consisting of C₁₋₁₀ alkyl, C₃₋₁₀cycloalky, C₆₋₁₅aryl, C₆₋₁₅arylC₁₋₆alkyl, a 5 to 15 membered heteroaryl, a 5 to 15 membered heteroarylC₁₋₆alkyl and a 5 to 15 membered heterocyclyl; wherein the C₁₋₁₀alkyl, C₃₋₁₀cycloalkyl, C₆₋₁₅aryl, a 5 to 15 membered heteroaryl, or a 5 to 15 membered heterocyclyl, whether alone or as part of a substituent group, is optionally substituted with one to three R⁴ substituent;
wherein each R⁴ is independently selected from the group consisting of hydroxy, halo, cyano, C₁₋₁₀alkoxy, C₁₋₁₀ alkyl, halogenatedC₁₋₁₀alkyl, C₃₋₁₀cycloalkyl, aminoC₁₋₁₀alkyl, C₁₋₁₀alkylamino, di(C₁₋₁₀)alkylamino, a 5 to 10 membered heterocyclyl, C₆₋₁₀aryl, a 5 to 10 membered heteroaryl, —NH₂, —C(O)R⁶, —C(O)OR⁶ and C₁₋₁₀alkylthio; wherein said C₁₋₁₀ alkoxy, C₁₋₁₀alkyl, C₃₋₁₀ cycloalkyl, 5 to 10 membered heterocyclyl, C₆₋₁₀ aryl, 5 to 10 membered heteroaryl, is optionally substituted with hydroxy, halogenatedC₁₋₃alkyl, cyano, halo, C₁₋₃ alkyl, C₁₋₃ alkoxy, —C(O)R⁶ or —C(O)OR⁶; and wherein R⁶ is selected from the group consisting of hydrogen, C₁₋₃ alkyl, and —NH₂;
R² is selected from the group consisting of hydrogen, —C(O)R⁸ and C₁₋₆alkyl; wherein the C₁₋₆alkyl is optionally substituted with 1 to 2 substituent independently selected from the group consisting of hydroxy, cyano, and halo;
alternatively, R¹ and R² are taken together with the nitrogen atom to which they are attached to form a five to seven membered heterocyclic ring which is optionally fused to a C₅₋₆aryl or a C₅₋₇ cycloalkyl; and wherein said C₅₋₆aryl or C₅₋₇ cycloalkyl is optionally substituted with 1 to 3 substituents independently selected from the group consisting of halo, cyano, C₁₋₃alkyl, C₁₋₃ alkoxy, halogenatedC₁₋₃alkyl, —C(O)R⁶ and —C(O)OR⁶;
R³ is selected from the group consisting of C₆₋₁₅ aryl, C₆₋₁₅arylC₁₋₆alkyl, C₆₋₁₅aryl-NH—C₁₋₆alkyl, a 5 to 15 membered heteroaryl, and a 5 to 15 membered heteroarylC₁₋₆alkyl; wherein the C₆₋₁₅ aryl or 5 to 15 membered heteroaryl, whether alone or as part of a substituent group is optionally substituted with 1 to 3 R⁴ substituents;
R⁷ is selected from the group consisting of C₁₋₁₀ alkyl and C₃₋₁₀ cycloalkyl; wherein the C₁₋₁₀ alkyl or C₃₋₁₀ cycloalkyl is optionally substituted with 1 to 3 R⁴ substituents;
R⁸ is selected from the group consisting of C₁₋₆ alkyl and C₃₋₇ cycloalkyl,
and enantiomers, stereoisomers, pro-drugs, solvates, and pharmaceutically acceptable salts thereof.

26. A compound according to claim 25, wherein
Q is selected from the group consisting of N and N⁺O⁻;
X is selected from the group consisting of N-(2-hydroxy-ethyl-phenyl)-amino-, N-(2-hydroxymethyl-5-trifluoromethyl-phenyl)-amino-, N-(2-hydroxymethyl-5-chloro-phenyl)-amino, (R)—N-ethyl-N-(napthy-1-yl)-amino-, N-(2-hydroxyethyl-phenyl)-amino-, N-ethyl-N-(naphth-1-yl)-amino-, N-(2-hydroxymethyl-4-fluoro-phenyl)-amino-, N-(2-hydroxymethyl-4-chloro-phenyl)-amino-, N-(2-methyl-5-methoxy-phenyl)-amino-, N-(hydroxyethyl)-N-(naphth-1-yl)-amino-, N-(2-hydroxymethyl-phenyl)-amino-, N-(2-methyl-4-methoxy-phenyl)-amino-, N-methyl-N-(naphth-1-yl)-amino-, N-methyl-N-(6-methoxy-naphth-1-yl)-amino-, N-ethyl-N-(5,6,7,8-tetrahydro-naphth-1-yl)-amino-, 2-(8-trifluoromethyl-1,2,3,4-tetrahydroisoquinolinyl), N-(2-methyl-5-trifluoromethyl-phenyl)-amino-, 2-methyl-5-methoxy-phenyl-oxy-, 2-(1,2,3,4-tetrahydroisoquinolinyl), N-(2-chloro-5-methoxy-phenyl)-amino-, N-(2-carboxyphenyl)-amino-, N-(2,5-dimethoxy-phenyl)-amino-, N-(2,5-dimethylphenyl)-amino-, N-methyl-N-(4-methoxy-naphth-1-yl)-amino-, N-methyl-N-(2-methyl-5-methoxy-phenyl)-amino-, N-methyl-N-(2,5-dimethyl-phenyl)-amino-, N-(2-ethyl-phenyl)-amino-, N-methyl-N-(2-methylphenyl)-amino-, (R)—N-methyl-N—(S)-1,2,3,4-tetrahydronaphth-1-yl)-amino-, 2-(8-methoxycarbonyl-1,2,3,4-tetrahydro-isoquinolinyl)-, 2-(7-fluoro-1,2,3,4-tetrahydro-isoquinolinyl)-, N-(2-methylphenyl)-amino-, N-methyl-N-(2-chloro-5-methoxy-phenyl)-amino-, N-methyl-N-(4-chloro-naphth-1-yl)-amino-, N-(cyanomethyl)-N-(naphth-1-y)-amino-, N-(2-methyl-5-fluoro-phenyl)-amino-, 2-(7-chloro-1,2,3,4-tetrahydro-isoquinolinyl), N-methyl-N-(2-methyl-naphth-1-yl)-amino-, N-(2-methyl-5-chloro-phenyl)-amino-, N-methyl-N-(1,2,3,4-tetrahydroquinolin-1-yl), 2-(5-chloro-1,2,3,4-tetrahydroisoquinolinyl), (S)—N-methyl-N—(R)-(1,2,3,4-tetrahydro-naphth-1-yl)-amino-, 2-hydroxyethyl-phenyl-oxy-, 1-(1,2,3,4-tetrahydro-quinolinyl), N-(2-methyl-5-phenyl-phenyl)-amino-, N-methyl-N-(2-methyl-5-chloro-phenyl)-amino-, N-methyl-(2,5-dimethoxy-phenyl)-amino-, N-(4-hydroxymethyl-pyrid-3-yl)-amino, N-methyl-N-(indan-4-yl), N-(2-cyano-phenyl-amino-, N-(2-aminomethyl-phenyl)-amino-, N-ethyl-N-(2,3-dimethyl-phenyl)-amino-, (S)—N-methyl-N—(S)-(1,2,3,4-tetrahydro-naphth-1-yl)-amino-, N-(methylcarbonyl)-N-(naphth-1-yl)-amino-, N-methyl-N-(2-methyl-4-methoxy-phenyl)-amino-, N-(3-methoxy-phenyl)-amino-, N-methyl-N-phenyl-amino-, (R)—N-methyl-N—(R)-(1,2,3,4-tetrahydro-naphth-1-yl)-amino-, N-ethyl-N-benzyl-amino-, benzyloxy-, N-methyl-N-(phenylethyl)-amino-, N-(indan-2-yl)-amino-, 2-(2,3-dihydro-1H-isoindolyl), N-methyl-N-(naphth-1-yl-methyl)-amino-, N-methyl-N-(phenyl-n-propyl)-amino, N-methyl-N-(2-furyl-methyl)-amino-, 1-(2,3-dihydro-1H-indolyl), N-methyl-N-(2-thienyl-methyl)-amino-, N-methyl-N-(indan-2-yl)-amino-, 2-(7-cyano-1,2,3,4-tetrahydro-isoquinolinyl), 2-(7-methoxy-carbonyl-1,2,3,4-tetrahydro-isoquinolinyl), 2-(6-methoxy-carbonyl -1,2,3,4-tetrahydro-isoquinolinyl), 2-(5-methoxy-carbonyl-1,2,3,4-tetrahydro-isoquinolinyl), 2-(7-methoxy-1,2,3,4-tetrahydro-isoquinolinyl), 2-(7-trifluoromethyl-1,2,3,4-tetrahydro-isoquinolinyl), 2-(6-trifluoromethyl-1,2,3,4-tetrahydro-isoquinolinyl), N-(naphth-1-yl)-amino, naphth-1-yl-oxy-, N-(naphth-1-yl)-amino-ethyl-oxy-, N-methyl-N-(2-methyl-5-isopropyl-phenyl)-amino-, 2-methyl-5-isopropyl-phenyl-oxy-, N-(methyl)-N-(5-(2-methyl-1,2,3,4-tetrahydro-isoquinolinyl)-amino-, N-(methyl)-N-(indol-4-yl)-amino, N-(methyl)-N-(2,6-dimethyl-phenyl)-amino-, N-(methyl)-N-(2-methyl-3-chloro-phenyl)-amino-, N-(methyl)-N-(7-methoxy-naphth-1-yl)-amino-, N-(methyl)-N-(2-methyl-3-methoxy-phenyl)-amino-, N-(methyl)-N-(5-methoxy-naphth-1-yl)-amino-, N-(2-methyl-naphth-1-yl)-amino-N-(2-trifluoromethyl-phenyl)-amino-, N-(4-indanyl)-amino-, N-(methyl)-N-(3-(4-methyl-biphenyl))-amino-, N-(methyl)-N-(benzyl)-amino-, N-(methyl)-N-(cyclohexyl)-amino-, N-(methyl)-N-(3-pyridyl-methyl)-amino-, N-pyrrolidinyl, 2-(6,7-dimethoxy-1,2,3,4-tetrahydro-isoquinolinyl), 2-(6-carboxy-1,2,3,4-tetrahydro-isoquinolinyl), 2-(5-carboxy-1,2,3,4-tetrahydro-isoquinolinyl), 2-(8-carboxy-1,2,3,4-tetrahydro-isoquinolinyl), 2-(7-carboxy-1,2,3,4-tetrahydro-isoquinolinyl), N-(naphth-1-yl)-amino-, N-(ethyl)-N-(naphth-1-yl)-amino-, N-(ethyl)-N-(quinazolin-4-yl)-amino-, N-(2-(4-methoxy-biphenyl))-amino-, N-(ethyl)-amino-, 2-(hydroxymethyl)-phenyl-oxy-, N-(2-methyl-5-methoxy-phenyl)-amino-, N-(2-methyl-4-hydroxymethyl-phenyl)-amino-, N-(3-hydroxymethyl-phenyl)-amino-, 2-formyl-5-methoxy-phenyl-oxy-, N-(2-methoxy-carbonyl-phenyl)-amino-, N-(2-amino-benzyl)-amino-, N-(2-methylcarbonyl-phenyl)-amino-, N-(2-methyl-5-hydroxy-phenyl)-amino, N-(2-aminocarbonyl-phenyl)-amino- and 2-ethoxycarbonyl-phenyl-oxy-;
Y is selected from the group consisting of hydrogen, hydroxy, chloro, methylthio, ethyl, methoxy, ethoxy, isopropoxy, benzyloxy, cyclopentyl-oxy and dimethylamino-ethoxy;
Z is selected from the group consisting of naphth-1-yl, phenyl, 2-fluorophenyl, 2-methylphenyl, 2-ethylphenyl, 2-isopropylphenyl, 2,6-dimethyl-phenyl, 2,6-dimethoxy-phenyl, 2,6-diethyl-phenyl, 2-chloro-6-methoxy-phenyl, 2,6-dimethyl-4-methoxy-phenyl, 2-(biphenyl), 3-thienyl, 3-pyridyl, 4-(3,5-dimethyl-isoxazolyl) and 2-(benzo[1,3]-dioxolyl);
R⁵ is selected from the group consisting of hydrogen, methyl and n-propyl;
and enantiomers, stereoisomers, pro-drugs, solvates, and pharmaceutically acceptable salts thereof.

27. A compound according to claim 25, wherein
Q is selected from the group consisting of N and N⁺O⁻;
X is selected from the group consisting of N-(2-hydroxy-ethyl-phenyl)-amino-, N-(2-hydroxymethyl-5-trifluoromethyl-phenyl)-amino-, N-(2-hydroxymethyl-5-chloro-phenyl)-amino, (R)—N-ethyl-N-(napthy-1-yl)-amino-, N-(2-hydroxyethyl-phenyl)-amino-, N-(ethyl)-N-(naphth-1-yl)-amino-, N-(2-hydroxymethyl-4-fluoro-phenyl)-amino-, N-(2-hydroxymethyl-4-chloro-phenyl)-amino-, N-(2-methyl-5-methoxy-phenyl)-amino-, N-(hydroxyethyl)-N-(naphth-1-yl)-amino-, N-(2-hydroxymethyl-phenyl)-amino-, N-(2-methyl-4-methoxy-phenyl)-amino-, N-methyl-N-(naphth-1-yl)-amino-, N-methyl-N-(6-methoxy-naphth-1-yl)-amino-, N-ethyl-N-(5,6,7,8-tetrahydro-naphth-1-yl)-amino-, 2-(8-trifluoromethyl-1,2,3,4-tetrahydroisoquinolinyl), N-(2-methyl-5-trifluoromethyl-phenyl)-amino-, 2-methyl-5-methoxy-phenyl-oxy-, 2-(1,2,3,4-tetrahydroisoquinolinyl), N-(2-chloro-5-methoxy-phenyl)-amino-, N-(2-carboxyphenyl)-amino-, N-(2,5-dimethoxy-phenyl)-amino-, N-(2,5-dimethylphenyl)-amino-, N-methyl-N-(4-methoxy-naphth-1-yl)-amino-, N-methyl-N-(2-methyl-5-methoxy-phenyl)-amino-, N-methyl-N-(2,5-dimethyl-phenyl)-amino-, N-(2-ethyl-phenyl)-amino-, N-methyl-N-(2-methylphenyl)-amino-, (R)—N-methyl-N—(S)-1,2,3,4-tetrahydronaphth-1-yl)-amino-, 2-(8-methoxycarbonyl-1,2,3,4-tetrahydro-isoquinolinyl)-, 2-(7-fluoro-1,2,3,4-tetrahydro-isoquinolinyl)-, N-(2-methylphenyl)-amino-, N-methyl-N-(2-chloro-5-methoxy-phenyl)-amino-, N-methyl-N-(4-chloro-naphth-1-yl)-amino-, N-(cyanomethyl)-N-(naphth-1-y)-amino-, N-(2-methyl-5-fluoro-phenyl)-amino-, 2-(7-chloro-1,2,3,4-tetrahydro-isoquinolinyl), N-methyl-N-(2-methyl-naphth-1-yl)-amino-, N-(2-methyl-5-chloro-phenyl)-amino-, N-methyl-N-(1,2,3,4-tetrahydroquinolin-1-yl), N-(hydroxyethyl)-N-(naphth-1-yl)-amino-, 2-(5-chloro-1,2,3,4-tetrahydroisoquinolinyl), (S)—N-methyl-N—(R)-(1,2,3,4-tetrahydro-naphth-1-yl)-amino-, N-methyl-N-(naphth-1-yl)-amino-, N-(2-methyl-5-methoxy-phenyl)-amino-, 2-hydroxyethyl-phenyl-oxy-, 1-(1,2,3,4-tetrahydro-quinolinyl), N-(2-methyl-5-phenyl-phenyl)-amino-, N-methyl-N-(2-methyl-5-chloro-phenyl)-amino-, N-methyl-(2,5-dimethoxy-phenyl)-amino-, N-(4-hydroxymethyl-pyrid-3-yl)-amino, N-methyl-N-(indan-4-yl), N-(2-cyano-phenyl-amino-, N-(2-aminomethyl-phenyl)-amino-, N-ethyl-N-(2,3-dimethyl-phenyl)-amino-, (S)—N-methyl-N—(S)-(1,2,3,4-tetrahydro-naphth-1-yl)-amino-, N-(methylcarbonyl)-N-(naphth-1-yl)-amino-, N-methyl-N-(2-methyl-4-methoxy-phenyl)-amino-, N-(3-methoxy-phenyl)-amino-, N-methyl-N-phenyl-amino- and(R)—N-methyl-N—(R)-(1,2,3,4-tetrahydro-naphth-1-yl)-amino-;
Y is selected from the group consisting of isopropoxy, methoxy, cyclopentyl-oxy, ethoxy, methylthio, ethyl, benzyloxy, chloro and dimethyl-amino-ethoxy;
Z is selected from the group consisting of 2,6-diethyl-phenyl, 2-chloro-6-methoxy-phenyl, 2,6-dimethyl-phenyl, 2,6-dimethyl-4-methoxy-phenyl, 2-methyl-phenyl, 2-ethyl-phenyl, 2-isopropyl-phenyl, 2,6-dimethoxy-phenyl, 2-fluoro-phenyl, 2,6-dimethyl-phenyl and naphth-1-yl;
R⁵ is selected from the group consisting of hydrogen and n-propyl;
and enantiomers, stereoisomers, pro-drugs, solvates, and pharmaceutically acceptable salts thereof.

28. A compound according to claim 25, wherein
Q is selected from the group consisting of N and N⁺O⁻;
X is selected from the group consisting of N-(2-hydroxy-ethyl-phenyl)-amino-, N-(2-hydroxymethyl-5-trifluoromethyl-phenyl)-amino-, N-(2-hydroxymethyl-5-chloro-phenyl)-amino, (R)—N-ethyl-N-(napthy-1-yl)-amino-, N-(2-hydroxyethyl-phenyl)-amino-, N-(ethyl)-N-(naphth-1-yl)-amino-, N-(2-hydroxymethyl-4-fluoro-phenyl)-amino-, N-(2-hydroxymethyl-4-chloro-phenyl)-amino-, N-(2-methyl-5-methoxy-phenyl)-amino-, N-(hydroxyethyl)-N-(naphth-1-yl)-amino-, N-(2-hydroxymethyl-phenyl)-amino-, N-methyl-N-(naphth-1-yl)-amino-, N-methyl-N-(6-methoxy-naphth-1-yl)-amino-, N-ethyl-N-(5,6,7,8-tetrahydro-naphth-1-yl)-amino-, 2-(8-trifluoromethyl-1,2,3,4-tetrahydroisoquinolinyl), N-(2-methyl-5-trifluoromethyl-phenyl)-amino-, 2-methyl-5-methoxy-phenyl-oxy-, 2-(1,2,3,4-tetrahydroisoquinolinyl), N-(2-chloro-5-methoxy-phenyl)-amino-, N-(2-carboxyphenyl)-amino-, N-(2,5-dimethoxy-phenyl)-amino-, N-(2,5-dimethylphenyl)-amino-, N-methyl-N-(4-methoxy-naphth-1-yl)-amino-, N-methyl-N-(2-methyl-5-methoxy-phenyl)-amino-, N-methyl-N-(2,5-dimethyl-phenyl)-amino-, N-(2-ethyl-phenyl)-amino-, N-methyl-N-(2-methylphenyl)-amino-, (R)—N-methyl-N—(S)-1,2,3,4-tetrahydronaphth-1-yl)-amino-, 2-(8-methoxycarbonyl-1,2,3,4-tetrahydro-isoquinolinyl)-, 2-(7-fluoro-1,2,3,4-tetrahydro-isoquinolinyl)-, N-(2-methyl-phenyl)-amino- and N-methyl-N-(2-chloro-5-methoxy-phenyl)-amino-;
Y is selected from the group consisting of isopropoxy, methoxy, cyclopentyloxy, ethoxy, methylthio, ethyl and benzyloxy;
Z is selected from the group consisting of Z is selected from the group consisting of 2,6-diethyl-phenyl, 2-chloro-6-methoxy-phenyl, 2,6-dimethyl-phenyl, 2,6-dimethyl-4-methoxy-phenyl, 2-methyl-phenyl and2-ethyl-phenyl;
R⁵ is hydrogen;
and enantiomers, stereoisomers, pro-drugs, solvates, and pharmaceutically acceptable salts thereof.

29. A compound selected from the group consisting of 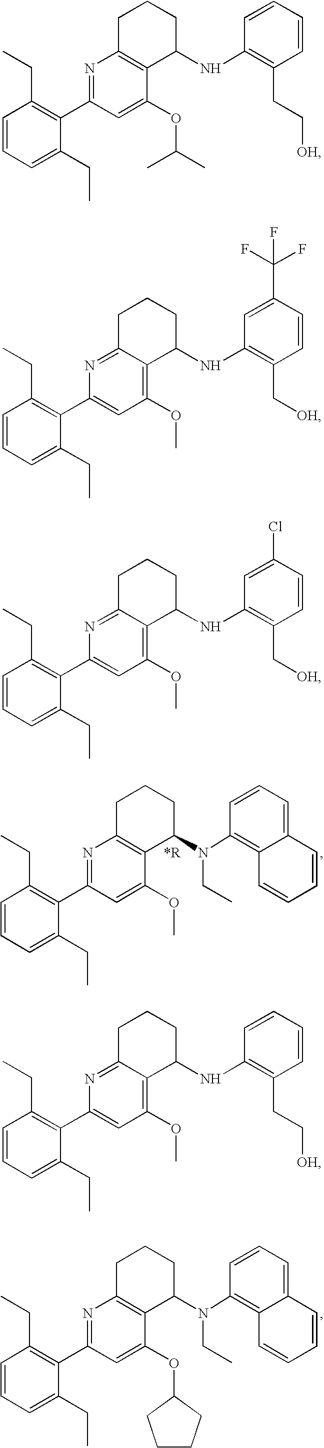 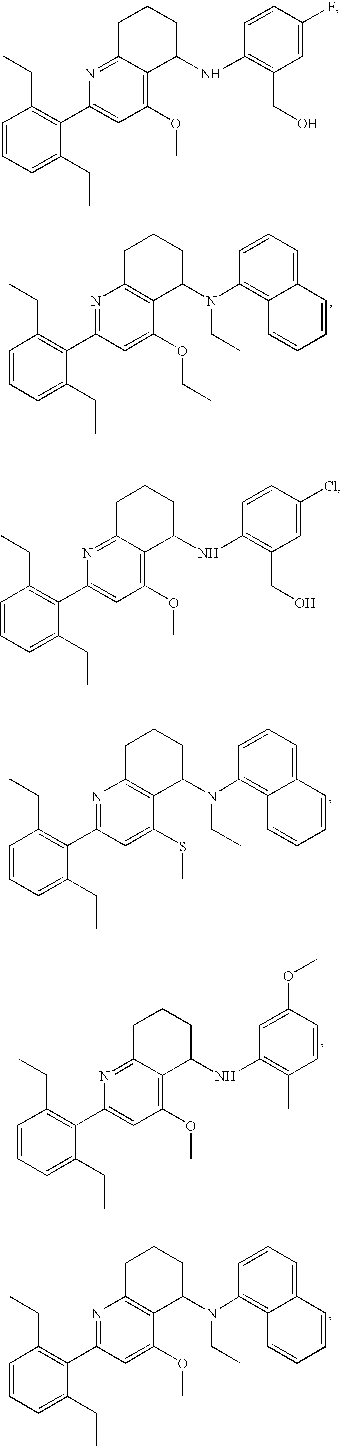 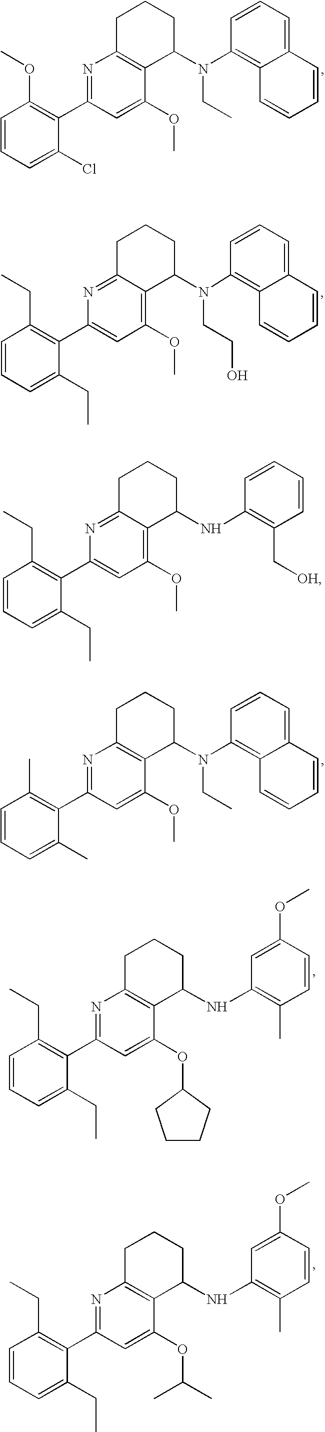 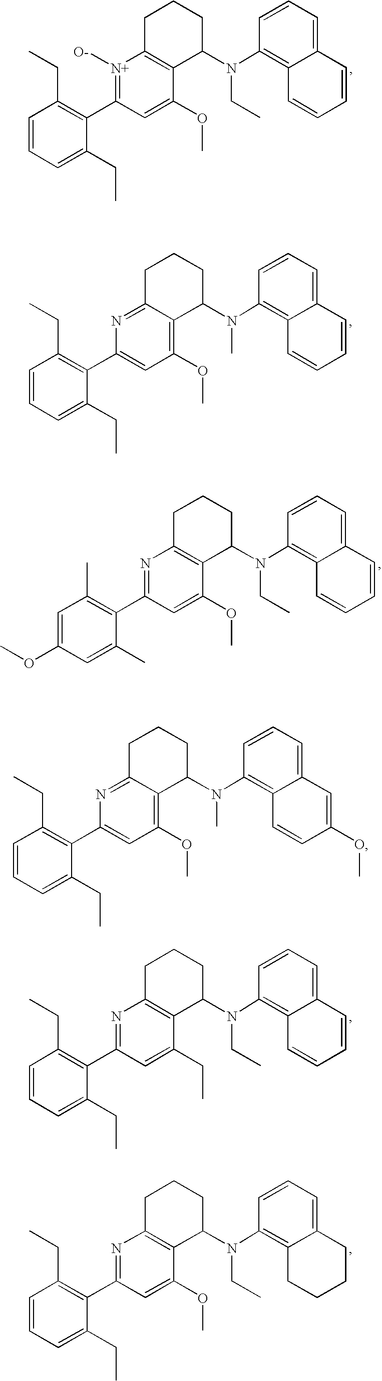 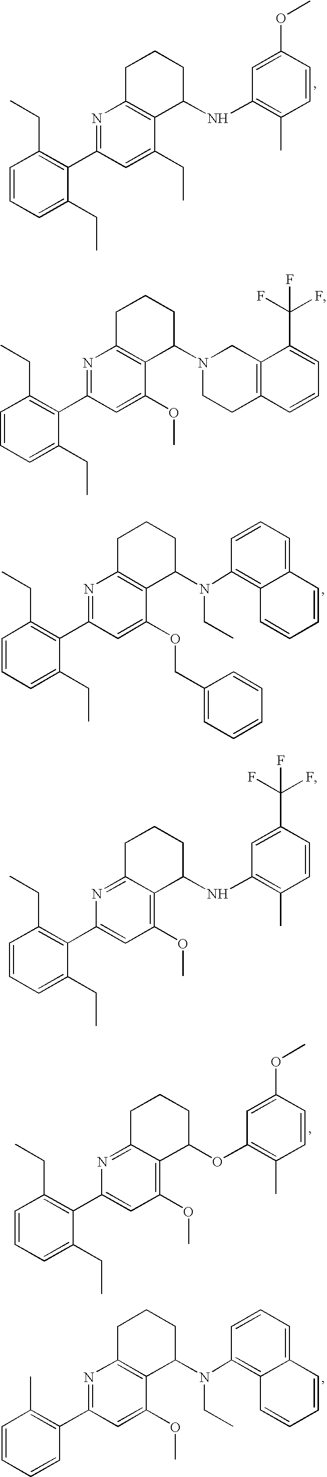 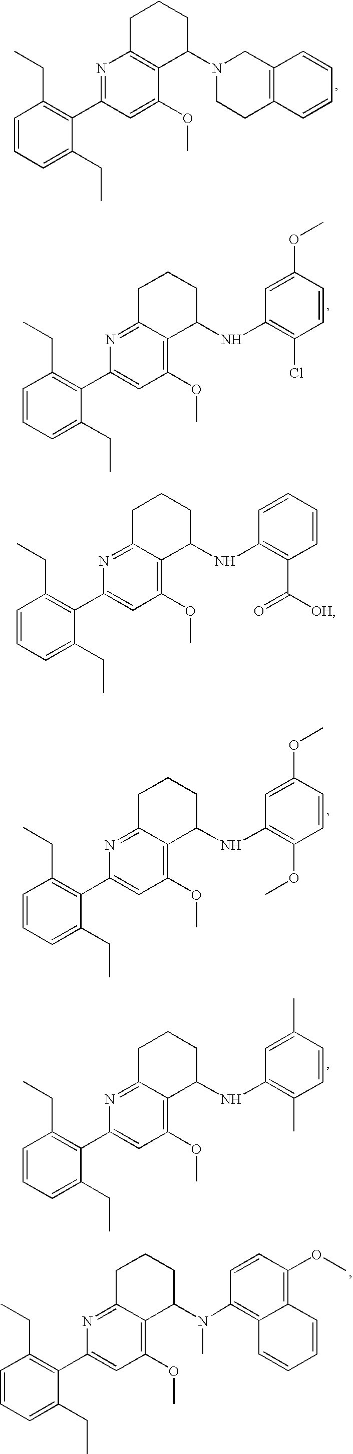 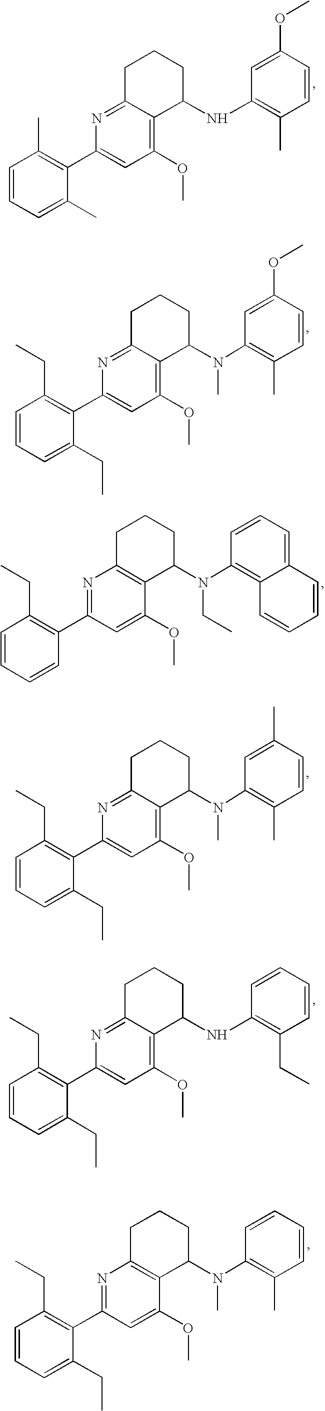 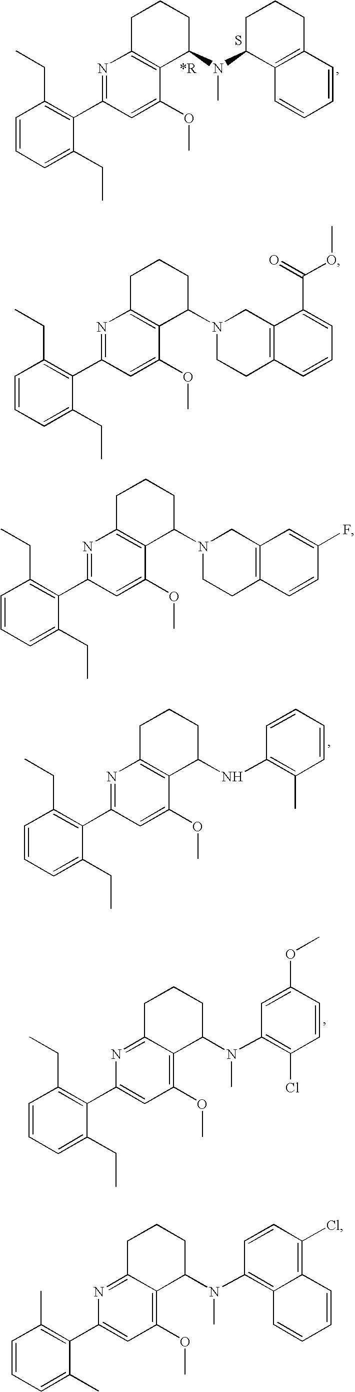 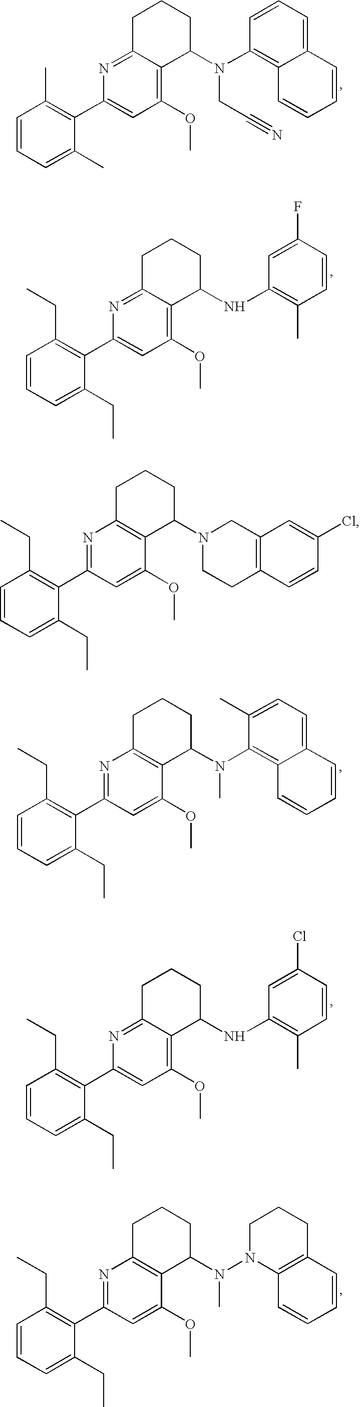 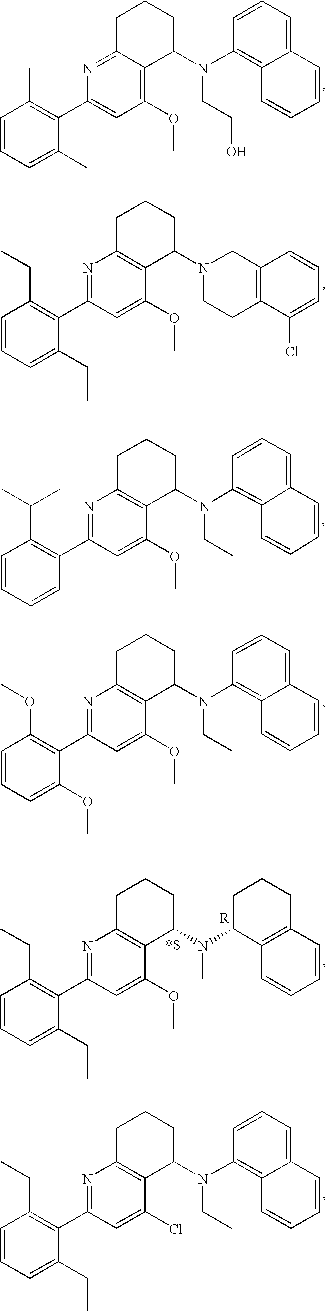 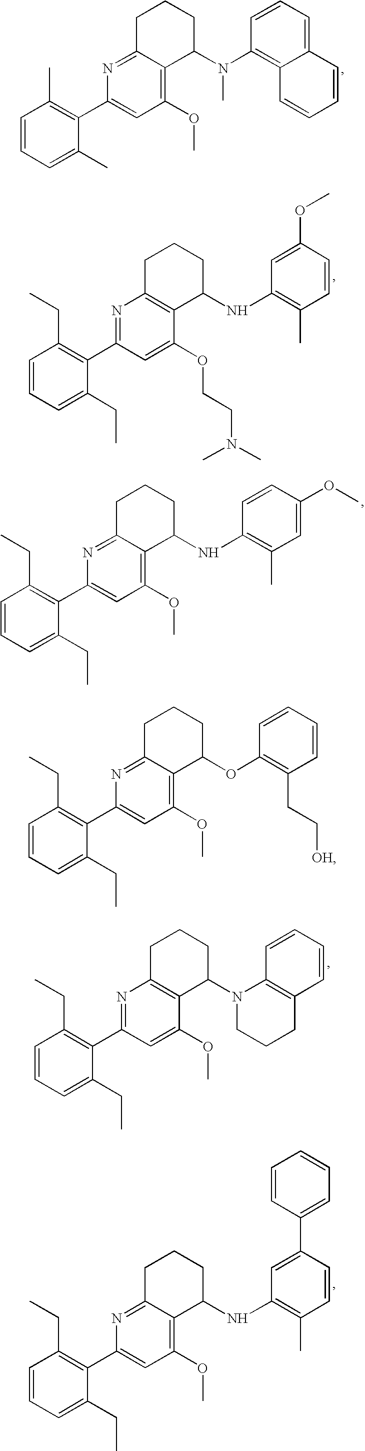 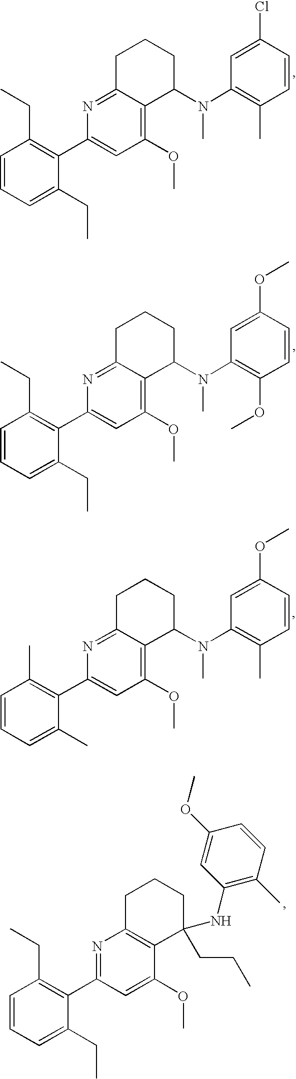 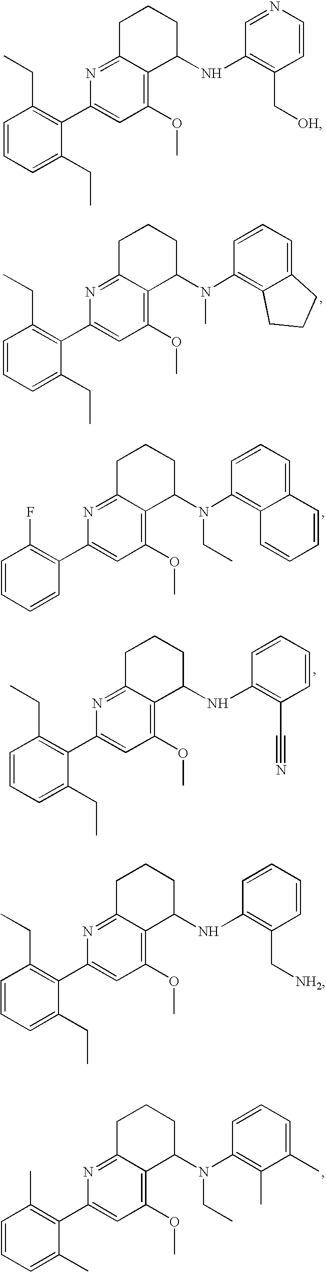 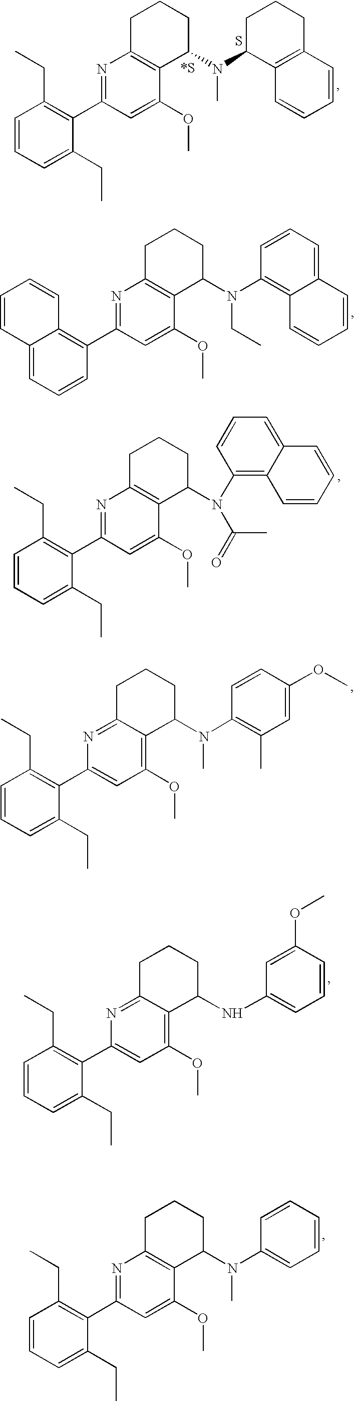 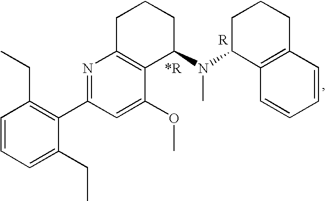
and enantiomers, stereoisomers, pro-drugs, solvates, and pharmaceutically acceptable salts thereof.

30. A composition, comprising: (a) at least one compound of claim 1 or a pharmaceutically acceptable salt thereof; and (b) at least one pharmaceutically acceptable carrier.

31. A method of treating an immune or inflammatory disease or condition in a patient in need thereof, comprising the step of administering to said patient an effective amount of a compound of claim 1 or a pharmaceutically acceptable salt thereof.

32. A method according to claim 31, wherein said immune or inflammatory disease or condition is selected from the group consisting of sepsis, rheumatoid arthritis, psoriasis, inflammatory bowel disease, immune complex diseases, systemic lupus erythematosus, lupus nephritis, Alzheimer's disease, ischemia/reperfusion injury, multiple sclerosis, myasthenia gravis, glomerulonephritis, chronic graft rejection, gingivitis, asthma, dermatitis, Guillain-Barre syndrome, myocardial infarct, pancreatitis, cystic fibrosis, atherosclerosis, fibrosis, allergies, diabetes type I, and combinations thereof.
